(19) United States
(12) Patent Application Publication
(10) Pub. No.: US 2007/0172844 A1
(43) Pub. Date: Jul. 26, 2007

(54) Individualized cancer treatments

(75) Inventors: Johnathan M. Lancaster, Tampa, FL (US); Joseph R. Nevins, Chapel Hill, NC (US)

Correspondence Address: MORRISON & FOERSTER LLP, 755 PAGE MILL RD, PALO ALTO, CA, 94304-1018, US

(73) Assignees: University of South Florida, Tampa, FL (US); Duke University, Durham, NC (US)

(21) Appl. No.: 11/541,165

(22) Filed: Sep. 28, 2006

Related U.S. Application Data
(60) Provisional application No. 60/721,213, filed on Sep. 28, 2005.
(60) Provisional application No. 60/731,335, filed on Oct. 28, 2005.
(60) Provisional application No. 60/778,769, filed on Mar. 3, 2006.
(60) Provisional application No. 60/779,163, filed on Mar. 3, 2006.
(60) Provisional application No. 60/779,473, filed on Mar. 6, 2006.

(51) Int. Cl. C12Q 1/68; C12M 1/34

(52) U.S. Cl. 435/6; 435/287.2

(57) ABSTRACT

The invention provides for compositions and methods for predicting an individual's responsitivity to cancer treatments and methods of treating cancer. In certain embodiments, the invention provides compositions and methods for predicting an individual's responsitivity to chemotherapeutics, including platinum-based chemotherapeutics, to treat cancers such as ovarian cancer. Furthermore, the invention provides for compositions and methods for predicting an individual's responsivity to salvage therapeutic agents. By predicting if an individual will or will not respond to platinum-based chemotherapeutics, a physician can reduce side effects and toxicity by administering a particular additional salvage therapeutic agent. This type of personalized medical treatment for ovarian cancer allows for more efficient treatment of individuals suffering from ovarian cancer. The invention also provides reagents, such as DNA microarrays, software and computer systems useful for personalizing cancer treatments, and provides methods of conducting a diagnostic business for personalizing cancer treatments.

### CROSS-REFERENCE TO RELATED APPLICATIONS

This application claims the priority benefit of the U.S. Provisional Application Ser. No. 60/721,213, filed Sep. 28, 2005; U.S. Provisional Application Ser. No. 60/731,335, filed Oct. 28, 2005; U.S. Provisional Application Ser. No. 60/778,769, filed Mar. 3, 2006; U.S. Provisional Application Ser. No. 60/779,163, filed Mar. 3, 2006; U.S. Provisional Application Ser. No. 60/779,473, filed Mar. 6, 2006, all of which are hereby incorporated by reference in their entirety.

### STATEMENT REGARDING FEDERALLY SPONSORED RESEARCH OR DEVELOPMENT

This invention was made with government support under NCI-U54 CA112952-02 and R01-CA106520 awarded by the National Cancer Institute. The government has certain rights in the invention.

### FIELD OF THE INVENTION

This invention relates to the use of gene expression profiling to determine whether an individual afflicted with cancer will respond to a therapy, and in particular to a therapeutic agents such as platinum-based agents. The invention also relates to the treatment of the individuals with the therapeutic agents. If the individual appears to be partially responsive or non-responsive to platinum-based therapy, then the individual's gene expression profile is used to determine which salvage agent should be used to further treat the individual to maximize cytotoxicity for the cancerous cells while minimizing toxicity for the individual.

### BACKGROUND OF THE INVENTION

Throughout this specification, reference numbering is sometimes used to refer to the full citation for the references, which can be found in the “Reference Bibliography” after the Examples section. The disclosure of all patents, patent applications, and publications cited herein are hereby incorporated by reference in their entirety for all purposes.

Cancer is considered to be a serious and pervasive disease. The National Cancer Institute has estimated that in the United States alone, one in three people will be afflicted with cancer during their lifetime. Moreover approximately 50% to 60% of people contracting cancer will eventually die from the disease. Lung cancer is one of the most common cancers with an estimated 172,000 new cases projected for 2003 and 157,000 deaths.³⁹ Lung carcinomas are typically classified as either small-cell lung carcinomas (SCLC) or non-small cell lung carcinomas (NSCLC). SCLC comprises about 20% of all lung cancers with NSCLC comprising the remaining approximately 80%. NSCLC is further divided into adenocarcinoma (AC)(about 30-35% of all cases), squamous cell carcinoma (SCC)(about 30% of all cases) and large cell carcinoma (LCC)(about 10% of all cases). Additional NSCLC subtypes, not as clearly defined in the literature, include adenosquamous cell carcinoma (ASCC), and bronchioalveolar carcinoma (BAC).

Lung cancer is the leading cause of cancer deaths worldwide, and more specifically non-small cell lung cancer accounts for approximately 80% of all disease cases.⁴⁰ There are four major types of non-small cell lung cancer, including adenocarcinoma, squamous cell carcinoma, bronchioalveolar carcinoma, and large cell carcinoma. Adenocarcinoma and squamous cell carcinoma are the most common types of NSCLC based on cellular morphology.⁴¹ Adenocarcinomas are characterized by a more peripheral location in the lung and often have a mutation in the K-ras oncogene.⁴² Squamous cell carcinomas are typically more centrally located and frequently carry p53 gene mutations.⁴³

One particularly prevalent form of cancer, especially among women, is breast cancer. The incidence of breast cancer, a leading cause of death in women, has been gradually increasing in the United States over the last thirty years. In 1997, it was estimated that 181,000 new cases were reported in the U.S. and that 44,000 people would die of breast cancer.⁴⁴⁻⁴⁵

Ovarian cancer is a leading cause of cancer death among women in the United States and Western Europe and has the highest mortality rate of all gynecologic cancers. Currently, platinum drugs are the most active agents in epithelial ovarian cancer therapy. ¹⁻³ Consequently, the standard treatment protocol used in the initial management of advanced-stage ovarian cancer is cytoreductive surgery, followed by primary chemotherapy with a platinum-based regimen that usually includes a taxane.⁴ Approximately 70% of patients (or individuals with ovarian cancer) will have a complete clinical response to this initial therapy, with absence of clinical or radiographic detectable residual disease and normalization of serum CA 125 levels.^{5,6} The remaining 30% of patients will demonstrate residual or progressive platinum-resistant disease. The inability to predict response to specific therapies is a major impediment to improving outcome for women with ovarian cancer. Empiric-based treatment strategies are used and result in many patients with chemo-resistant disease receiving multiple cycles of often toxic therapy without success before the lack of efficacy is identified. In the course of these empiric treatments, patients may experience significant toxicities, compromise to bone marrow reserves, detriment to quality of life, and delay in the initiation of therapy with active agents. Moreover, the lack of active therapeutic agents for patients with platinum-resistant disease limits treatment options. As such, many patients receive chemotherapy with little or no benefit.

Patients with platinum-resistant recurrent disease are treated with salvage agents such as topotecan, liposomal doxorubicin, gemcitabine, etoposide and ifosfamide. Response rates for patients with platinum-resistant disease range are generally less than 20%, with the potential for significant cumulative toxicities that include thrombocytopenia, peripheral neuropathy, palmar-plantar erythodysthesia (PPE), and secondary leukemias.⁴⁶⁻⁴⁸ Response rates are dependent on clinical factors such as the response to initial platinum therapy, the disease-free interval before recurrence, previous agents used, existing cumulative toxicities, and the patient's performance status. Although choice of salvage agent is made based-upon all of these factors, no reliable clinical or biologic predictor of response to therapy exists, such that the majority of patients are treated somewhat empirically.

The clinical heterogeneity of ovarian cancer, resulting from the acquisition of multiple genetic alterations that contribute to the development of the tumor, underlies the heterogeneity of response to chemotherapy.⁷ Although a variety of gene alterations have been identified, no single gene marker can reliably predict response to therapy and outcome.⁸⁻¹² Recent advances in the use of DNA microarrays, that allow global assessment of gene expression in a single sample, have shown that expression profiles can provide molecular phenotyping that identifies distinct classifications not evident by traditional histopathological methods.¹³⁻²⁰

Throughout treatment for ovarian cancer, prolongation of survival and the successful maintenance of quality of life remain important goals. Improving the ability to manage the disease by optimizing the use of existing drugs and/or developing new agents is essential in this endeavor. To this end, individualizing treatments by identifying patients that will respond to specific agents will potentially increase response rates, and limit the incidence and severity of toxicities that not only limit quality of life, but ability to tolerate further therapies.

Therefore, it would be highly desirable to able to identify whether an individual or a patient with cancer, and in particular with ovarian cancer, will be responsive to platinum-based therapy. It would also be highly desirable to determine which salvage therapy agent could be used that would minimize the toxicity to the individual and yet be effective in eliminating cancerous cells. Finally, it would be desirable to predict which anti-cancer agents will effectively treat the cancer in an individual to provide a personalized treatment plan.

### BRIEF SUMMARY OF THE INVENTION

The invention provides, in one aspect, a method for identifying whether an individual with ovarian cancer will be responsive to a platinum-based therapy by (a) obtaining a cellular sample from the individual; (b) analyzing said sample to obtain a first gene expression profile; (c) comparing said first gene expression profile to a platinum chemotherapy responsivity predictor set of gene expression profiles; and (d) identifying whether said individual will be responsive to a platinum-based therapy.

In another aspect, the invention provides a method of identifying whether an individual will benefit from the administration of an additional cancer therapeutic other than a platinum-based therapeutic comprising: (a) obtaining a cellular sample from the individual; (b) analyzing said sample to obtain a first gene expression profile; (c) comparing said first gene expression profile to a platinum chemotherapy responsivity predictor set of gene expression profiles to identify whether said individual will be responsive to a platinum-based therapy; (d) if said individual is an incomplete responder to platinum based therapy, then comparing the first gene expression profile to a set of gene expression profiles that is capable of predicting responsiveness to other cancer therapy agents; thereby identifying whether said individual would benefit from the administration of one or more cancer therapy agents.

In yet another aspect, the invention provides a method of treating an individual with ovarian cancer comprising: (a) obtaining a cellular sample from the individual; (b) analyzing said sample to obtain a first gene expression profile; (c) comparing said first gene expression profile to a platinum chemotherapy responsivity predictor set of gene expression profiles to identify whether said individual will be responsive to a platinum-based therapy; (d) if said individual is a complete responder or incomplete responder, then administering an effective amount of platinum-based therapy to the individual; (e) if said individual is predicted to be an incomplete responder to platinum based therapy, then comparing the first gene expression profile to a set of gene expression profiles that is predictive of responsivity to additional cancer therapeutics to identify to which additional cancer therapeutic the individual would be responsive; and (f) administering to said individual an effective amount of one or more of the additional cancer therapeutic that was identified in step (e); thereby treating the individual with ovarian cancer.

In yet another aspect, the invention provides a method of reducing toxicity of chemotherapeutic agents in an individual with cancer comprising: (a) obtaining a cellular sample from the individual; (b) analyzing said sample to obtain a first gene expression profile; (c) comparing said first gene expression profile to a set of gene expression profiles that is capable of predicting responsiveness to common chemotherapeutic agents; and (d) administering to the individual an effective amount of that agent.

In yet another aspect, the invention provides for a gene chip for predicting an individual's responsivity to a platinum-based therapy comprising the gene expression profile of at least 5 genes selected from Table 2.

In yet another aspect, the invention provides for a gene chip for predicting an individual's responsivity to a platinum-based therapy comprising the gene expression profile of at least 10 genes selected from Table 2.

In yet another aspect, the invention provides for a gene chip for predicting an individual's responsivity to a platinum-based therapy comprising the gene expression profile of at least 20 genes selected from Table 2.

In yet another aspect, the invention provides for a kit comprising a gene chip for predicting an individual's responsivity to a platinum-based therapy and a set of instructions for determining an individual's responsivity to platinum-based chemotherapy agents.

In yet another aspect, the invention provides for a gene chip for predicting an individual's responsivity to a salvage therapy agent comprising the gene expression profile of at least 5 genes selected from Table 4 or Table 5.

In yet another aspect, the invention provides for a gene chip for predicting an individual's responsivity to a salvage therapy agent comprising the gene expression profile of at least 10 genes selected from Table 4 or Table 5.

In yet another aspect, the invention provides for a gene chip for predicting an individual's responsivity to a salvage therapy agent comprising the gene expression profile of at least 20 genes selected from Table 4 or Table 5.

In yet another aspect, the invention provides for a kit comprising a gene chip for predicting an individual's responsivity to a salvage therapy agent and a set of instructions for determining an individual's responsivity to salvage therapy agents.

In yet another aspect, the invention provides for a computer readable medium comprising gene expression profiles comprising at least 5 genes from any of Tables 2, 4 or 5.

In yet another aspect, the invention provides for a computer readable medium comprising gene expression profiles comprising at least 15 genes from Tables 2, 4 or 5.

In yet another aspect, the invention provides for a computer readable medium comprising gene expression profiles comprising at least 25 genes from Tables 2, 4 or 5.

In yet another aspect, the invention provides a method for estimating or predicting the efficacy of a therapeutic agent in treating an individual afflicted with cancer. In one aspect, the method comprises: (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more statistical tree models applied to the values of the metagenes, wherein each model includes one or more nodes, each node representing a metagene, each node including a statistical predictive probability of tumor sensitivity to the therapeutic agent, thereby estimating the efficacy of a therapeutic agent in an individual afflicted with cancer. In certain embodiments, step (a) comprises extracting a nucleic acid sample from the sample from the subject. In certain embodiments, the method further comprising: (d) detecting the presence of pathway deregulation by comparing the expression levels of the genes to one or more reference profiles indicative of pathway deregulation, and (e) selecting an agent that is predicted to be effective and regulates a pathway deregulated in the tumor. In certain embodiments said pathway is selected from RAS, SRC, MYC, E2F, and β-catenin pathways.

In yet another aspect, the invention provides a method for estimating the efficacy of a therapeutic agent in treating an individual afflicted with cancer. In one aspect, the method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more binary regression models applied to the values of the metagenes, wherein each model includes a statistical predictive probability of tumor sensitivity to the therapeutic agent, thereby estimating the efficacy of a therapeutic agent in an individual afflicted with cancer.

In yet another aspect, the invention provides a method of treating an individual afflicted with cancer, said method comprising: (a) estimating the efficacy of a plurality of therapeutic agents in treating an individual afflicted with cancer according to the methods if the invention; (b) selecting a therapeutic agent having the high estimated efficacy; and (c) administering to the subject an effective amount of the selected therapeutic agent, thereby treating the subject afflicted with cancer.

In yet another aspect, the invention provides a therapeutic agent having the high estimated efficacy is one having an estimated efficacy in treating the subject of at least 50%. In certain embodiments, the invention provides a therapeutic agent having the high estimated efficacy is one having an estimated efficacy in treating the subject of at least 80%.

In certain embodiments, the tumor is selected from a breast tumor, an ovarian tumor, and a lung tumor. In certain embodiments, the therapeutic agent is selected from docetaxel, paclitaxel, topotecan, adriamycin, etoposide, fluorouracil (5-FU), and cyclophosphamide, or any combination thereof.

In certain embodiments, the therapeutic agent is docetaxel and wherein the cluster of genes comprises at least 10 genes from metagene 1. In certain embodiments, the therapeutic agent is paclitaxel, and wherein the cluster of genes comprises at least 10 genes from metagene 2. In certain embodiments, wherein the therapeutic agent is topotecan, and wherein the cluster of genes comprises at least 10 genes from metagene 3. In certain embodiments, wherein the therapeutic agent is adriamycin, and wherein the cluster of genes comprises at least 10 genes from metagene 4. In certain embodiments, wherein the therapeutic agent is etoposide, and wherein the cluster of genes comprises at least 10 genes from metagene 5. In certain embodiments, wherein the therapeutic agent is fluorouracil (5-FU), and wherein the cluster of genes comprises at least 10 genes from metagene 6. In certain embodiments, wherein the therapeutic agent is cyclophosphamide and wherein the cluster of genes comprises at least 10 genes from metagene 7.

In certain embodiments, at least one of the metagenes is metagene 1, 2, 3, 4, 5, 6, or 7. In certain embodiments, the cluster of genes corresponding to at least one of the metagenes comprises 3 or more genes in common to metagene 1, 2, 3, 4, 5, 6, or 7. In certain embodiments, the cluster of genes corresponding to at least one metagene comprises 5 or more genes in common to metagene 1, 2, 3, 4, 5, 6, or 7. In certain embodiments, the cluster of genes corresponding to at least one metagene comprises at least 10 genes, wherein half or more of the genes are common to metagene 1, 2, 3, 4, 5, 6, or 7.

In certain embodiments, each cluster of genes comprises at least 3 genes. In certain embodiments, each cluster of genes comprises at least 5 genes. In certain embodiments, each cluster of genes comprises at least 7 genes. In certain embodiments, each cluster of genes comprises at least 10 genes. In certain embodiments, each cluster of genes comprises at least 12 genes. In certain embodiments, each cluster of genes comprises at least 15 genes. In certain embodiments, each cluster of genes comprises at least 20 genes.

In certain embodiments, the expression level of multiple genes in the tumor biopsy sample is determined by quantitating nucleic acids levels of the multiple genes using a DNA microarray.

In certain embodiments, at least one of the metagenes shares at least 50% of its defining genes in common with metagene 1, 2, 3, 4, 5, 6, or 7. In certain embodiments, at least one of the metagenes shares at least 75% of its defining genes in common with metagene 1, 2, 3, 4, 5, 6, or 7. In certain embodiments, at least one of the metagenes shares at least 90% of its defining genes in common with metagene 1, 2, 3, 4, 5, 6, or 7. In certain embodiments, at least one of the metagenes shares at least 95% of its defining genes in common with metagene 1, 2, 3, 4, 5, 6, or 7. In certain embodiments, at least one of the metagenes shares at least 98% of its defining genes in common with metagene 1, 2, 3, 4, 5, 6, or 7.

In certain embodiments, the cluster of genes for at least two of the metagenes share at least 50% of their genes in common with one of metagenes 1, 2, 3, 4, 5, 6, or 7. In certain embodiments, the cluster of genes for at least two of the metagenes share at least 75% of their genes in common with one of metagenes 1, 2, 3, 4, 5, 6, or 7. In certain embodiments, the cluster of genes for at least two of the metagenes share at least 90% of their genes in common with one of metagenes 1, 2, 3, 4, 5, 6, or 7. In certain embodiments, the cluster of genes for at least two of the metagenes share at least 95% of their genes in common with one of metagenes 1, 2, 3, 4, 5, 6, or 7. In certain embodiments, the cluster of genes for at least two of the metagenes share at least 98% of their genes in common with one of metagenes 1, 2, 3, 4, 5, 6, or 7.

In yet another aspect, the invention provides a method for defining a statistical tree model predictive of tumor sensitivity to a therapeutic agent, the method comprising: (a) determining the expression level of multiple genes in a set of cell lines, wherein the set of cell lines includes cell lines resistant to the therapeutic agent and cell lines sensitive to the therapeutic agent; (b) identifying clusters of genes associated with sensitivity or resistance to the therapeutic agent by applying correlation-based clustering to the expression level of the genes; (c) defining one or more metagenes, wherein each metagene is defined by extracting a single dominant value using singular value decomposition (SVD) from a cluster of genes associated with sensitivity or resistance; and (d) defining a statistical tree model, wherein the model includes one or more nodes, each node representing a metagene from step (c), each node including a statistical predictive probability of tumor sensitivity or resistance to the agent, thereby defining a statistical tree model indicative of tumor sensitivity to a therapeutic. In certain embodiments, the method further comprising: (e) determining the expression level of multiple genes in a tumor biopsy samples from human subjects (f) calculating predicted probabilities of effectiveness of a therapeutic agent for tumor biopsy samples; and (g) comparing these probabilities to clinical outcomes of said subjects to determine the accuracy of the predicted probabilities, thereby validating the statistical tree model in vivo. In certain embodiments, the method further comprises: (e) obtaining an expression profile from a tumor biopsy sample from the subject; and (f) determining an estimate of the efficacy of a therapeutic agent or combination of agents in treating cancer in an individual by averaging the predictions of one or more of the statistical models applied to the expression profile of the tumor biopsy sample. In certain embodiments, step (d) is reiterated at least once to generate additional statistical tree models.

In certain embodiments, clinical outcomes are selected from disease-specific survival, disease-free survival, tumor recurrence, therapeutic response, tumor remission, and metastasis inhibition.

In certain embodiments, each model comprises two or more nodes. In certain embodiments, each model comprises three or more nodes. In certain embodiments, each model comprises four or more nodes.

In certain embodiments, the model predicts tumor sensitivity to an agent with at least 80% accuracy.

In certain embodiments, the model predicts tumor sensitivity to an agent with greater accuracy than clinical variables alone.

In certain embodiments, the clinical variables are selected from age of the subject, gender of the subject, tumor size of the sample, stage of cancer disease, histological subtype of the sample and smoking history of the subject.

In certain embodiments, the cluster of genes comprises at least 3 genes. In certain embodiments, the cluster of genes comprises at least 5 genes. In certain embodiments, the cluster of genes comprises at least 10 genes. In certain embodiments, the cluster of genes comprises at least 15 genes. In certain embodiments, the correlation-based clustering is Markov chain correlation-based clustering or K-means clustering.

In yet another aspect, the invention provides a method of estimating the efficacy of a therapeutic agent in treating cancer in an individual, said method comprising: (a) obtaining an expression profile from a tumor biopsy sample from the subject; and (b) calculating probabilities of effectiveness from an in vivo validated signature applied to the expression profile of the tumor biopsy sample.

In certain embodiments, the therapeutic agent is selected from docetaxel, paclitaxel, topotecan, adriamycin, etoposide, fluorouracil (5-FU), and cyclophosphamide

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a gene expression pattern associated with platinum response. Part A (the left panel) shows results from a leave-one-out cross validation of training set (blue=square=Incomplete Responders, red=triangle=Responders). The right panel shows a ROC curve of the training set. Part B shows that the validation of the platinum response prediction was based on a cut-off of 0.47 predicted probability of response as determined by ROC curve.

FIG. 2 depicts a prediction of oncogenic pathway deregulation and drug sensitivity in ovarian cancer cell lines. Panel A shows the predicted probability of pathway activation. For each of the graphs in panels B and C, the low Src is indicated in blue and the high Src is indicated in red in ovarian tumors (n=119). Panel B shows a Kaplan-Meier survival analysis demonstrating relationship of Src and E2F3 pathway activation and survival of patients that demonstrated an incomplete response to primary platinum therapy. Panel C shows a Kaplan-Meier survival analysis demonstrating relationship of Src and E2F3 pathway activation and survival of patients that demonstrated a complete response to primary platinum therapy.

FIG. 3 depicts a prediction of Src and E2F3 pathway deregulation predicts sensitivity to pathway-specific drugs. Panel A shows pathway predictions (red=high and blue=low probability) in ovarian cancer cell lines. Panel B depicts sensitivity of cell lines to Src inhibitor (SU6656)(left) and CDK inhibitor (CYC202/R-Roscovitine)(right). The growth inhibition assays are plotted as percent inhibition of proliferation versus probability of pathway activation (Src and E2F3).

FIG. 4 depicts sensitivity of ovarian cancer cell lines to combinations of pathway-specific and cytotoxic drugs as a function of pathway deregulation. The top panel shows proliferation inhibition of cisplatin (green), SU6656 (blue) and combination of SU6656 and cisplatin (red) plotted as a function of probability of Src pathway activation. Panel B is similar to panel A but with CYC202/R-Roscovitine (blue), cisplatin (green), and combination of CYC202/Roscovitine and cisplatin (red) with E2F3 pathway activation.

FIG. 5 depicts potential application of platinum response and pathway prediction in the treatment of patients with ovarian cancer.

FIG. 6 depicts a pair of graphs. The first graph (A) illustrates topotecan response predictions from the metagene tree model. Estimates and approximate 95% confidence intervals for topotecan response probabilities for each patient. Each patient is predicted in an out-of-sample cross validation based on a model completely regenerated from the data of the remaining patients. Patients indicated in red are those that had a topotecan response and those in blue are non-responders. The interval estimates for a few cases that stand out are wide, representing uncertainty due to disparities among predictions coming from individual tree models that are combined in the overall prediction. The second graph (B) illustrates a Receiver Operating Characteristic (ROC) curve depicting the accuracy of the prediction of response to topotecan therapy. This is a plot of the true positive rate against the false positive rate for varying cut-points of predicting response to platinum-based therapy. The curve is represented by the line, the closer the curve follows the left axis followed by the top border of the ROC space, the more accurate the assay. The red numbers corresponds to sensitivity and specificity of the indicated probability used to determine prediction of complete responders and incomplete responders based on genomic profile predictions used in FIG. 6. Thus the response indicates a capacity to achieve up to 80% sensitivity with 83% specificity in predicting topotecan responders. False positive rate (1—specificity) is represented on the X axis, and the True positive rate (sensitivity) is represented on the Y axis.

FIG. 7 depicts pathway-specific gene expression profiles were used to predict pathway status in 48 ovarian cancers. Hierarchical clustering of pathway activity in samples of human lung cancer. Prediction of Src, β-catenin, Myc, p63, PI3 kinase, E2F1, akt, E2F3, and Ras pathway status for responder and non responder tumor samples were independently determined using supervised binary regression analysis as described in Bild, et al.³⁶ Patterns in the tumor pathway predictions were identified by hierarchical clustering.

FIG. 8 depicts a graph illustrating the sensitivity to pathway specific drugs. The degree of proliferation response is displayed for each cell line in response to single agent topotecan, single agent Src inhibitor (SU6656), and combination treatment with topotecan and SU6656. The degree of proliferation response was plotted as a function of probability of Src pathway activation. Cells were treated either with 20 micromolar Src inhibitor (SU6656) alone, 20 micromolar Src inhibitor (SU6656)+0.3 micromolar topotecan, or 0.3 micromolar topotecan alone for 96 hours. Proliferation was assayed using a standard MTS tetrazolium colorimetric method.

FIG. 9 depicts a series of graphs illustrating the sensitivity to pathway specific activity to topotecan dose response in the NCI-60 cell lines. Predicted pathway activity of the NCI-60 cell lines were plotted against the dose response of topatecan. Degree of Topotecan dose response was plotted as a function of probability of (A) Src, (B) β-catenin, and (C) PI3 Kinase pathway activation in the NCI-60 cell lines.

FIG. 10 shows the development of a predictor of topotecan sensitivity. Panel A shows gene expression profile used to selected to predict topotecan response. Panel B shows the topotecan response predictions developed from patient data. Estimates and approximate 95% confidence intervals for topotecan response probabilities for each patient. Each patient is predicted in an out-of-sample cross validation based on a model completely regenerated from the data of the remaining patients. Patients indicated in red are those that had a topotecan response and those in blue are non-responders.

FIG. 11 depicts a prediction of salvage therapy response using cell line developed expression signatures. Panel A shows the prediction for topotecan. Panel B shows the prediction for taxol. Panel C shows the prediction for docetaxel. Panel D shows the prediction for adriamycin.

FIG. 12 depicts patterns of predicted sensitivity to salvage chemotherapies in ovarian patients. Panel A shows a heatmap. Panel B shows regressions. Panel C shows regressions.

FIG. 13 depicts profiles of oncogenic pathway deregulation in relation to salvage agent sensitivity. Part A left panel shows patterns of pathway activity were predicted in samples following sorting based on predicted topotecan sensitivity. Prediction of Src, β-catenin, Myc, p63, PI3 kinase, EM, akt, E2173, and Ras pathway status were independently determined using supervised binary regression analysis as described in Bild, et al.³⁶ The right panel depicts a relationship between topotecan sensitivity and Src pathway deregulation. Part B left panel shows patterns of pathway activity were predicted in samples following sorting based on predicted adriamycin sensitivity. The right panel shows a relationship between adriamycin sensitivity and E217 pathway deregulation.

FIG. 14 depicts the relationship between salvage agent resistance and sensitivity to pathway-specific drugs in ovarian cancer cell lines. Part A shows patterns of pathway activity were predicted in the cell line samples following sorting based on predicted topotecan sensitivity. Part B shows the relationship between topotecan sensitivity and sensitivity to Src inhibition. Part C show patterns of pathway activity were predicted in the cell line samples following sorting based on predicted adriamycin sensitivity. Part D shows the relationship between adriamycin sensitivity and sensitivity to Roscovitine.

FIG. 15 is a diagram that shows opportunities for selection of appropriate therapy for advanced stage ovarian cancer patients.

FIGS. 16A-16E show a gene expression signature that predicts sensitivity to docetaxel. (A) Strategy for generation of the chemotherapeutic response predictor. (B) Top panel—Cell lines from the NCI-60 panel used to develop the in vitro signature of docetaxel sensitivity. The figure shows a statistically significant difference (Mann Whitney U test of significance) in the IC₅₀/GI₅₀ and LC₅₀ of the cell lines chosen to represent the sensitive and resistant subsets. Bottom Panel—Expression plots for genes selected for discriminating the docetaxel resistant and sensitive NCI-60 cell lines, depicted by color coding with blue representing the lowest level and red the highest. Each column in the figure represents individual samples. Each row represents an individual gene, ordered from top to bottom according to regression coefficients. (C) Top Panel—Validation of the docetaxel response prediction model in an independent set of lung and ovarian cancer cell line samples. A collection of lung and ovarian cell lines were used in a cell proliferation assay to determine the 50% inhibitory concentration (IC₅₀) of docetaxel in the individual cell lines. A linear regression analysis demonstrates a statistically significant (p<0.01, log rank) relationship between the IC₅₀ of docetaxel and the predicted probability of sensitivity to docetaxel. Bottom panel—Validation of the docetaxel response prediction model in another independent set of 29 lung cancer cell line samples (Gemma A, Geo accession number: GSE 4127). A linear regression analysis demonstrates a very significant (p<0.001, log rank) relationship between the IC₅₀ of docetaxel and the predicted probability of sensitivity to docetaxel. (D) Left Panel—A strategy for assessment of the docetaxel response predictor as a function of clinical response in the breast neoadjuvant setting. Middle panel—Predicted probability of docetaxel sensitivity in a collection of samples from a breast cancer single agent neoadjuvant study. Twenty of twenty four samples (91.6%) were predicted accurately using the cell line based predictor of response to docetaxel. Right panel—A single variable scatter plot demonstrating a significance test of the predicted probabilities of sensitivity to docetaxel in the sensitive and resistant tumors (p<0.001, Mann Whitney U test of significance). (E) Left Panel—A strategy for assessment of the docetaxel response predictor as a function of clinical response in advanced ovarian cancer. Middle panel—Predicted probability of docetaxel sensitivity in a collection of samples from a prospective single agent salvage therapy study. Twelve of fourteen samples (85.7%) were predicted accurately using the cell line based predictor of response to docetaxel. Right panel—A single variable scatter plot demonstrating statistical significance (p<0.01, Mann Whitney U test of significance).

FIGS. 17A-17C show the development of a panel of gene expression signatures that predict sensitivity to chemotherapeutic drugs. (A) Gene expression patterns selected for predicting response to the indicated drugs. The genes involved the individual predictors are shown in Table 5. (B) Independent validation of the chemotherapy response predictors in an independent set of cancer cell lines³⁷ that have dose response and Affymetrix expression data.³⁸ A single variable scatter plot demonstrating a significance test of the predicted probabilities of sensitivity to any given drug in the sensitive and resistant cell lines (p value, Mann Whitney U test of significance). Red symbols indicate resistant cell lines, and blue symbols indicate those that are sensitive. (C) Prediction of single agent therapy response in patient samples using in vitro cell line based expression signatures of chemosensitivity. In each case, red represents non-responders (resistance) and blue represents responders (sensitivity). The left panel shows the predicted probability of sensitivity to topotecan when compared to actual clinical response data (n=48), the middle panel demonstrates the accuracy of the adriamycin predictor in a cohort of 122 samples (Evans W, GSE650 and GSE651). The right panel shows the predictive accuracy of the cell line based paclitaxel predictor when used as a salvage chemotherapy in advanced ovarian cancer (n=35). The positive and negative predictive values for all the predictors are summarized in Table 6.

FIGS. 18A-18B show the prediction of response to combination therapy. (A) Left Panel—Strategy for assessment of chemotherapy response predictors in combination therapy as a function of pathologic response. Middle panel—Prediction of patient response to neoadjuvant chemotherapy involving paclitaxel, 5-flourouracil (5-FU), adriamycin, and cyclophosphamide (TFAC) using the single agent in vitro chemosensitivity signatures developed for each of these drugs. Right Panel—Prediction of response (38 non-responders, 13 responders) employing a combined probability predictor assessing the probability of all four chemosensitivity signatures in 51 patients treated with TFAC chemotherapy shows statistical significance (p<0.0001, Mann Whitney) between responders (blue) and non-responders (red). Response was defined as a complete pathologic response after completion of TFAC neoadjuvant therapy. (B) Left Panel—Prediction of patient response (n=45) to adjuvant chemotherapy involving 5-FU, adriamycin, and cyclophosphamide (FAC) using the single agent in vitro chemosensitivity predictors developed for these drugs. Middle panel—Prediction of response (34 responders, 11 non responders) employing a combined probability predictor assessing the probability of all four chemosensitivity signatures in 45 patients treated with FAC chemotherapy. Right panel—Kaplan Meier survival analysis for patients predicted to be sensitive (blue curve) or resistant (red curve) to FAC adjuvant chemotherapy.

FIG. 19 shows patterns of predicted sensitivity to common chemotherapeutic drugs in human cancers. Hierarchical clustering of a collection of breast (n=171), lung cancer (n=91) and ovarian cancer (n=119) samples according to patterns of predicted sensitivity to the various chemotherapeutics. These predictions were then plotted as a heatmap in which high probability of sensitivity/response is indicated by red, and low probability or resistance is indicated by blue.

FIGS. 20A-20B show the relationship between predicted chemotherapeutic sensitivity and oncogenic pathway deregulation. (A) Left Panel—Probability of oncogenic pathway deregulation as a function of predicted docetaxel sensitivity in a series of lung cancer cell lines (red=sensitive, blue=resistant). Right panel—Probability of oncogenic pathway deregulation as a function of predicted topotecan sensitivity in a series of ovarian cancer cell lines (red=sensitive, blue=resistant). (B) Left Panel—The lung cancer cell lines showing an increased probability of PI3 kinase were also more likely to respond to a PI3 kinase inhibitor (LY-294002)(p=0.001, log-rank test)), as measured by sensitivity to the drug in assays of cell proliferation. Further, those cell lines predicted to be resistant to docetaxel were more likely to be sensitive to PI3 kinase inhibition (p<0.001, log-rant test) Right panel—The relationship between Src pathway deregulation and topotecan resistance can be demonstrated in a set of 13 ovarian cancer cell lines. Ovarian cell lines that are predicted to be topotecan resistant have a higher likelihood of Src pathway deregulation and there is a significant linear relationship (p=0.001, log rank) between the probability of topotecan resistance and sensitivity to a drug that inhibits the Src pathway (SU6656).

FIG. 21 shows a scheme for utilization of chemotherapeutic and oncogenic pathway predictors for identification of individualized therapeutic options.

FIGS. 22A-22C show a patient-derived docetaxel gene expression signature predicts response to docetaxel in cancer cell lines. (A) Top panel—A ROC curve analysis to show the approach used to define a cut-off, using docetaxel as an example. Middle panel—A t-test plot of significance between the probability of docetaxel sensitivity and IC 50 for docetaxel sensitive in cell lines, shown by histologic type. Bottom panel—A linear regression analysis showing the significant correlation between predicted intro sensitivity and actual sensitivity (IC50 for docetaxel), in lung and ovarian cancer cell lines. (B) Generation of a docetaxel response predictor based on patient data that was then validated in a leave on out cross validation and linear regression analyses (p-value obtained by log-rank), evaluated against the IC₅₀ for docetaxel in two NCI-60 cell line drug screening experiments. (C) A comparison of predictive accuracies between a predictor for docetaxel generated from the cell line data (left panel, accuracy: 85.7%) and a predictor generated from patients treatment data (right panel, accuracy: 64.3%) shows the relative inferiority of the latter approach, when applied to an independent dataset of ovarian cancer patients treated with single agent docetaxel.

FIGS. 23A-23C show the development of gene expression signatures that predict sensitivity to a panel of commonly used chemotherapeutic drugs. Panel A shows the gene expression models selected for predicting response to the indicated drugs, with resistant lines on the left, sensitive on the right for each predictor. Panel B shows the leave one out cross validation accuracy of the individual predictors. Panel C demonstrates the results of an independent validation of the chemotherapy response predictors in an-independent set of cancer cell lines³⁷ shown as a plot with error bars (blue-sensitive, red-resistant).

FIG. 24 shows the specificity of chemotherapy response predictors. In each case, individual predictors of response to the various cytotoxic drugs was plotted against cell lines known to be sensitive or sensitive to a given chemotherapeutic agent (e.g., adriamycin, paclitaxel).

FIG. 25 shows the absolute probabilities of response to various chemotherapies in human lung and breast cancer samples.

FIGS. 26A-26C show the relationships in predicted probability of response to chemotherapies in breast (Panel A), lung (Panel B) and ovarian cancer (Panel C). In each case, a regression analysis (log rank) of predicted probability of response of two drugs is shown.

FIG. 27 shows a gene expression based signature of PI3 kinase pathway deregulation. Image intensity display of expression levels for genes that most differentiate control cells expressing GFP from cells expressing the oncogenic activity of PI3 kinase. The expression value of genes composing each signature is indicated by color, with blue representing the lowest value and red representing the highest level. The panel below shows the results of a leave one out cross validation showing a reliable differentiation between GFP controls (blue) and cells expressing PI3 kinase (red).

FIGS. 28A-28C show the relationship between oncogenic pathway deregulation and chemosensitivity patterns (using docetaxel as an example). (A) Probability of oncogenic pathway deregulation as a function of predicted docetaxel sensitivity in the NCI-60 cell line panel (red=sensitive, blue=resistant). (B) Linear regression analysis (log-rank test of significance) to identify relationships between predicted docetaxel sensitivity or resistance and deregulation of PI3 kinase, E2F3, and Src pathways. (C) A non-parametric t-test of significance demonstrating a significant difference in docetaxel sensitivity, between those cell lines predicted to be either pathway deregulated (>50% probability, red) or quiescent (<50% probability, blue), shown for both E2F and PI3 kinase pathways.

FIG. 29 shows a scatter plot showing a linear regression analysis that identifies a statistically significant correlation between probability of docetaxel resistance and PI3 Kinase pathway activation in an independent cohort of 17 non-small cell lung cancer cell lines.

FIG. 30 shows a functional block diagram of general purpose computer system 3000 for performing the functions of the software provided by the invention.

### BRIEF DESCRIPTION OF THE TABLES

Table 1 depicts clinico-pathologic characteristics of ovarian cancer samples analyzed.

Table 2 lists the 100 genes that contribute the most weight in the prediction and that appeared most often within the models for platinum-based responsivity predictor set.

Table 3 depicts quantitative analysis of gene ontology categories represented in genes that predict platinum response. The number of occurrences of all biological process Gene Ontology (GO) annotations in the list of genes selected to predict platinum response was counted. The 20 most significant annotations are shown in order of decreasing significance. The middle column indicates the number of genes annotated with a GO annotation out of a total of 100 genes selected to predict platinum response. The In (Bayes Factor) column represents the Bayes factor, a measure of significance when comparing the prevalence of the annotation in the selected genes compared against its prevalence in the entire human genome. The Bayes factor is the ratio of the posterior odds of two binomial models, where one measures the probability that the prevalence of annotations differs between gene lists, and the other measures the probability that the prevalence is the same, normalized by the priors.

Table 4 lists the predictor set to predict responsivity to topotecan.

Table 5 lists the predictor set for commonly used chemotherapeutics.

Table 6 is a summary of the chemotherapy response predictors—validations in cell line and patient data sets.

Table 7 shows an enrichment analysis shows that a genomic-guided response prediction increases the probability of a clinical response in the different data sets studied.

Table 8 shows the accuracy of genomic-based chemotherapy response predictors is compared to previously reported predictors of response.

Table 9 lists the genes that constitute the predictor of PI3 kinase activation.

### DETAILED DESCRIPTION OF THE INVENTION

An individual who has ovarian cancer frequently has progressed to an advanced stage before any symptoms appear. The standard treatment for advanced stage (e.g., Stage III/IV) cancer is to combine cytosurgery (e.g., “debulking” the individual of the tumor) and to administer an effective amount of a platinum-based treatment. In some cases, carboplatin or cisplatin is administered. Other non-limiting alternatives to carboplatin and cisplatin are oxaliplatin and nedaplatin. Taxane is sometimes administered with the carboplatin or cisplatin. However, the platinum based treatment is not always effective for all patients. Thus, physicians have to consider alternative treatments to combat the ovarian cancer. Salvage therapy agents can be used as one alternative treatment. The salvage therapy agents include but are not limited to topotecan, etoposide, adriamycin, doxorubicin, gemcitabine, paclitaxel, docetaxel, and taxol. The difficulty with administering one or more salvage therapy agent is that not all individuals with ovarian cancer will respond favorably to the salvage therapy agent selected by the physician. Frequently, the administration of one or more salvage therapy agent results in the individual becoming even more ill from the toxicity of the agent and the cancer still persists. Due to the cytotoxic nature of the salvage therapy agent, the individual is physically weakened and his/her immunologically compromised system cannot generally tolerate multiple rounds of “trial and error” type of therapy. Hence a treatment plan that is personalized for the individual is highly desirable.

The inventors have described gene expression profiles associated with ovarian cancer development, surgical debulking, response to therapy, and survival.²¹⁻²⁷ Further, the inventors have applied genomic methodologies to identify gene expression patterns within primary tumors that predict response to primary platinum-based chemotherapy. This analysis has been coupled with gene expression signatures that reflect the deregulation of various oncogenic signaling pathways to identify unique characteristics of the platinum-resistant cancers that can guide the use of these drugs in patients with platinum-resistant disease. The invention thus provides integrating gene expression profiles that predict platinum-response and oncogenic pathway status as a strategy for developing personalized treatment plans for individual patients.

Definitions

“Platinum-based therapy” and “platinum-based chemotherapy” are used interchangeably herein and refers to agents or compounds that are associated with platinum.

As used herein, “array” and “microarray” are interchangeable and refer to an arrangement of a collection of nucleotide sequences in a centralized location. Arrays can be on a solid substrate, such as a glass slide, or on a semi-solid substrate, such as nitrocellulose membrane. The nucleotide sequences can be DNA, RNA, or any permutations thereof. The nucleotide sequences can also be partial sequences from a gene, primers, whole gene sequences, non-coding sequences, coding sequences, published sequences, known sequences, or novel sequences.

A “complete response” (CR) is defined as a complete disappearance of all measurable and assessable disease or, in the absence of measurable lesions, a normalization of the CA-125 level following adjuvant therapy. An individual who exhibits a complete response is known as a “complete responder.”

An “incomplete response” (IR) includes those who exhibited a “partial response” (PR), had “stable disease” (SD), or demonstrated “progressive disease” (PD) during primary therapy.

A “partial response” refers to a response that displays 50% or greater reduction in the product obtained from measurement of each bi-dimensional lesion for at least 4 weeks or a drop in the CA-125 by at least 50% for at least 4 weeks.

“Progressive disease” refers to response that is a 50% or greater increase in the product from any lesion documented within 8 weeks of initiation of therapy, the appearance of any new lesion within 8 weeks of initiation of therapy, or any increase in the CA-125 from baseline at initiation of therapy.

“Stable disease” was defined as disease not meeting any of the above criteria.

“Effective amount” refers to an amount of a chemotherapeutic agent that is sufficient to exert a biological effect in the individual. In most cases, an effective amount has been established by several rounds of testing for submission to the FDA. It is desirable for an effective amount to be an amount sufficient to exert cytotoxic effects on cancerous cells.

“Predicting” and “prediction” as used herein does not mean that the event will happen with 100% certainty. Instead it is intended to mean the event will more likely than not happen.

As used herein, “individual” and “subject” are interchangeable. A “patient” refers to an “individual” who is under the care of a treating physician. In one embodiment, the subject is a male. In one embodiment, the subject is a female.

General Techniques

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (ointly referred to herein as “Harlow and Lane”), Beaucage et al. eds., Current Protocols in Nucleic Acid Chemistry John Wiley & Sons, Inc., New York, 2000) and Casarett and Doull's Toxicology The Basic Science of Poisons, C. Klaassen, ed., 6th edition (2001).

Methods for Predicting Responsiveness to Platinum-Based Therapy

The invention provides methods and compositions for predicting an individual's responsiveness to a platinum-based therapy. In one embodiment, the individual has ovarian cancer. In another embodiment, the individual has advanced stage (e.g., Stage III/IV) ovarian cancer. In other embodiments, the individual has early stage ovarian cancer whereby cellular samples from the early stage ovary cancer are obtained from the individual. For the individuals with advanced ovarian cancer, one form of primary treatment practiced by treating physicians is to remove as much of the ovarian tumor as possible, a practice sometime known as “debulking.” In many cases, the individual is also put on a treatment plan that involves a form of platinum-based therapy (e.g., carboplatin or cisplatin) either with or without taxane.

The ovarian tumor that is removed is a potential source of cellular sample for nucleic acids to be used in a gene expression profiling. The cellular sample can come from tumor sample either from biopsy or surgery for debulking. In one alternative, the cellular sample comes from ascites surrounding the tumor tissue. The cellular sample is used as a source of nucleic acid for gene expression profiling.

The cellular sample is then analyzed to obtain a first gene expression profile. This can be achieved any number of ways. One method that can be used is to isolate RNA (e.g., total RNA) from the cellular sample and use a publicly available microarray systems to analyze the gene expression profile from the cellular sample. One microarray that may be used is Affymetrix Human U133A chip. One of skill in the art follows the standard directions that come with a commercially available microarray. Other types of microarrays be may be used, for example, microarrays using RT-PCR for measurement. Other sources of microarrays include, but are not limited to, Stratagene (e.g., Universal Human Microarray), Genomic Health (e.g., Oncotype DX chip), Clontech (e.g., Atlas™ Glass Microarrays), and other types of Affymetrix microarrays. In one embodiment, the microarray comes from an educational institution or from a collaborative effort whereby scientists have made their own microarrays. In other embodiments, customized microarrays, which include the particular set of genes that are particularly suitable for prediction, can be used.

Once a first gene expression profile has been obtained from the cellular sample, then it is used to compare with a platinum chemotherapy responsivity predictor set of gene expression profiles.

Platinum-based Therapy Responsivity Predictor Set of Gene Expression Profiles

A platinum-based therapy responsitivity predictor set was created as detailed in Example 1. A binary logistic regression model analysis and a stochastic regression model search, called Shotgun Stochastic Search (SSS), was used to determine platinum response predictions models in the training set of 83 samples. The predictive analysis evaluated regression models linking log values of observed expression levels of small numbers of genes to platinum response and debulking status. From the 5000 regression models that identify a total of 1727 genes, Table 2 lists the 100 genes that contribute the most weight in the prediction and that appeared most often within the models. The full list of 1727 genes is posted on the web site. The predictive accuracy for the platinum-based therapy responsitivity predictor set was tested using the “leave-one-out” cross-validation approach whereby the analysis is repeated performed where one sample is left out at each reanalysis and the response to therapy is predicted for that case.

Thus, one of skill in art uses the platinum-based therapy responsitivity predictor set as detailed in Example 1 to determine whether the first gene expression profile, obtained from the individual or patient with ovarian cancer will be responsive to the a platinum-based therapy. If the individual is a complete responder, then a platinum-based therapy agent will be administered in an effective amount, as determined by the treating physician. If the complete responder stops being a complete responder, as does happen in a certain percentage of time, then the first gene expression profile is then analyzed for responsivity to a salvage agent to determine which salvage agent should be administered to most effectively combat the cancer while minimizing the toxic side effects to the individual. If the individual is an incomplete responder, then the individual's gene expression profile can be further analyzed for responsivity to a salvage agent to determine which salvage agent should be administered.

The use of the platinum-based therapy responsitivity predictor set in its entirety is contemplated, however, it is also possible to use subsets of the predictor set. For example, a subset of at least 5 genes can be used for predictive purposes. Alternatively, at least 10 or 15 genes from the platinum-based therapy responsitivity predictor set can also be used.

Thus, in this manner, an individual can be diagnosed for responsiveness to platinum-based therapy. In certain embodiments, the methods of the application are performed outside of the human body. In addition, an individual can be diagnosed to determine if they will be refractory to platinum-based therapy such that additional therapeutic intervention, such as salvage therapy treatment, can be started.

Methods of Predicting Responsivity to Salvage Agents

For the individuals that appear to be incomplete responders to platinum-based therapy or for those individuals who have ceased being complete responders, an important step in the treatment is to determine what other additional cancer therapies might be given to the individual to best combat the cancer while minimizing the toxicity of these additional agents.

In one aspect, the additional therapy is a salvage agent. Salvage agents that are contemplated include, but are not limited to, topotecan, adriamycin, doxorubicin, cytoxan, cyclophosphamide, gemcitabine, etoposide, ifosfamide, paclitaxel, docetaxel, and taxol. In another aspect, the first gene expression profile from the individual with ovarian cancer is analyzed and compared to gene expression profiles (or signatures) that are reflective of deregulation of various oncogenic signal transduction pathways. In one embodiment, the additional cancer therapeutic agent is directed to a target that is implicated in oncogenic signal transduction deregulation. Such targets include, but are not limited to, Src, myc, beta-catenin and E2F3 pathways. Thus, in one aspect, the invention contemplates using an inhibitor that is directed to one of these targets as an additional therapy for ovarian cancer. One of skill in the art will be able to determine the dosages for each specific inhibitor since the inhibitor must under rigorous testing to pass FDA regulations before it can be used in treating humans.

As shown in Example 1, the teachings herein provide a gene expression model that predicts response to platinum-based therapy was developed using a training set of 83 advanced stage serous ovarian cancers, and tested on a 36-sample external validation set. In parallel, expression signatures that define the status of oncogenic signaling pathways were evaluated in 119 primary ovarian cancers and 12 ovarian cancer cell lines. In an effort to increase chemo-sensitivity, pathways shown to be activated in platinum-resistant cancers were subject to targeted therapy in ovarian cell lines.

The inventors have observed that gene expression profiles identified patients with ovarian cancer likely to be resistant to primary platinum-based chemotherapy, with greater than 80% accuracy. In patients with platinum-resistant disease, the expression signatures were consistent with activation of Src and Rb/E2F pathways, components of which were successfully targeted to increase response in ovarian cancer cell lines. Thus, the inventors have defined a strategy for treatment of patients with advanced stage ovarian cancer that utilizes therapeutic stratification based on predictions of response to chemotherapy, coupled with prediction of oncogenic pathway deregulation as a method to direct the use of targeted agents.

As shown in Example 2, the predictor set to determine responsitivity to topotecan is shown in Table 4. As with the platinum-based predictor set, not all of the genes in the topotecan predictor must be used. A subset comprising at least 5, 10, or 15 genes may be used a predictor set to determine responsivity to topotecan.

In addition to using gene expression profiles obtained from tumor samples taken during surgery to debulk individuals with ovarian cancer, it is also possible to generate a predictor set for predicting responsivity to common chemotherapy agents by using publicly available data. Numerous websites exist that share data obtained from microarray analysis. In one embodiment, gene expression profiling data obtained from analysis of 60 cancerous cells lines, known herein as NCI-60, can be used to generate a training set for predicting responsivity to cancer therapy agents. The NCI-60 training set can be validated by the same type of “Leave-one-out” cross-validation as described earlier.

The predictor sets for the other salvage therapy agents are shown in Table 5. These predictor sets are used as a reference set to compare the first gene expression profile from an individual with ovarian cancer to determine if she will be responsive to a particular salvage agent. In certain embodiments, the methods of the application are performed outside of the human body.

Method of Treating Individuals with Ovarian Cancer

This methods described herein also includes treating an individual afflicted with ovarian cancer. This is accomplished by administering an effective amount of a platinum-based therapy to those individual who will be responsive to such therapy. In the instance where the individual is predicted to be a non-responder, a physician may decide to administer salvage therapy agent alone. In most instances, the treatment will comprise a combination of a platinum-based therapy and a salvage agent. In one embodiment, the treatment will comprise a combination of a platinum-based therapy and an inhibitor of a signal transduction pathway that is deregulated in the individual with ovarian cancer.

In one aspect, platinum-based therapy is administered in an effective amount by itself (e.g., for complete responders). In another embodiment, the platinum-based therapy and a salvage agent are administered in an effective amount concurrently. In another embodiment, the platinum-based therapy and a salvage agent are administered in an effective amount in a sequential manner. In yet another embodiment, the salvage therapy agent is administered in an effective amount by itself. In yet another embodiment, the salvage therapy agent is administered in an effective amount first and then followed concurrently or step-wise by a platinum-based therapy.

Methods of Predicting/Estimating the Efficacy of a Therapeutic Agent in Treating a Individual Afflicted with Cancer

One aspect of the invention provides a method for predicting, estimating, aiding in the prediction of, or aiding in the estimation of, the efficacy of a therapeutic agent in treating a subject afflicted with cancer. In certain embodiments, the methods of the application are performed outside of the human body.

One method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more statistical tree models applied to the values of the metagenes, wherein each model includes one or more nodes, each node representing a metagene, each node including a statistical predictive probability of tumor sensitivity to the therapeutic agent, thereby estimating the efficacy of a therapeutic agent in a subject afflicted with cancer. Another method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more binary regression models applied to the values of the metagenes, wherein each model includes a statistical predictive probability of tumor sensitivity to the therapeutic agent, thereby estimating the efficacy of a therapeutic agent in a subject afflicted with cancer.

In one embodiment, the predictive methods of the invention predict the efficacy of a therapeutic agent in treating a subject afflicted with cancer with at least 70% accuracy. In another embodiment, the methods predict the efficacy of a therapeutic agent in treating a subject afflicted with cancer with at least 80% accuracy. In another embodiment, the methods predict the efficacy of a therapeutic agent in treating a subject afflicted with cancer with at least 85% accuracy. In another embodiment, the methods predict the efficacy of a therapeutic agent in treating a subject afflicted with cancer with at least 90% accuracy. In another embodiment, the methods predict the efficacy of a therapeutic agent in treating a subject afflicted with cancer with at least 70%, 80%, 85% or 90% accuracy when tested against a validation sample. In another embodiment, the methods predict the efficacy of a therapeutic agent in treating a subject afflicted with cancer with at least 70%, 80%, 85% or 90% accuracy when tested against a set of training samples. In another embodiment, the methods predict the efficacy of a therapeutic agent in treating a subject afflicted with cancer with at least 70%, 80%, 85% or 90% accuracy when tested on human primary tumors ex vivo or in vivo.

(A) Tumor Sample

In one embodiment, the predictive methods of the invention comprise determining the expression level of genes in a tumor sample from the subject, preferably a breast tumor, an ovarian tumor, and a lung tumor. In one embodiment, the tumor is not a breast tumor. In one embodiment, the tumor is not an ovarian tumor. In one embodiment, the tumor is not a lung tumor. In one embodiment of the methods described herein, the methods comprise the step of surgically removing a tumor sample from the subject, obtaining a tumor sample from the subject, or providing a tumor sample from the subject. In one embodiment, the sample contains at least 40%, 50%, 60%, 70%, 80% or 90% tumor cells. In preferred embodiments, samples having greater than 50% tumor cell content are used. In one embodiment, the tumor sample is a live tumor sample. In another embodiment, the tumor sample is a frozen sample. In one embodiment, the sample is one that was frozen within less than 5, 4, 3, 2, 1, 0.75, 0.5, 0.25, 0.1, 0.05 or less hours after extraction from the patient. Preferred frozen sample include those stored in liquid nitrogen or at a temperature of about −80 C or below.

(B) Gene Expression

The expression of the genes may be determined using any methods known in the art for assaying gene expression. Gene expression may be determined by measuring MRNA or protein levels for the genes. In a preferred embodiment, an mRNA transcript of a gene may be detected for determining the expression level of the gene. Based on the sequence information provided by the GenBankTm database entries, the genes can be detected and expression levels measured using techniques well known to one of ordinary skill in the art. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can be also carried out on a DNA array. The use of an array is preferable for detecting the expression level of a plurality of the genes. As another example, the sequences can be used to construct primers for specifically amplifying the polynucleotides in, e.g., amplification-based detection methods such as reverse-transcription based polymerase chain reaction (RT-PCR). Furthermore, the expression level of the genes can be analyzed based on the biological activity or quantity of proteins encoded by the genes.

Methods for determining the quantity of the protein includes immunoassay methods. Paragraphs 98-123 of U.S. Patent Pub No. 2006-0110753 provide exemplary methods for determining gene expression. Additional technology is described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; as well as WO 95/21265; WO 96/31622; WO 97/10365; WO 97/27317; EP 373 203; and EP 785 280.

In one exemplary embodiment, about 1-50 mg of cancer tissue is added to a chilled tissue pulverizer, such as to a BioPulverizer H tube (Bio101 Systems, Carlsbad, Calif.). Lysis buffer, such as from the Qiagen Rneasy Mini kit, is added to the tissue and homogenized. Devices such as a Mini-Beadbeater (Biospec Products, Bartlesville, Okla.) may be used. Tubes may be spun briefly as needed to pellet the garnet mixture and reduce foam. The resulting lysate may be passed through syringes, such as a 21 gauge needle, to shear DNA. Total RNA may be extracted using commercially available kits, such as the Qiagen RNeasy Mini kit. The samples may be prepared and arrayed using Affymetrix U133 plus 2.0 GeneChips or Affymetrix U133A GeneChips.

In one embodiment, determining the expression level of multiple genes in a tumor sample from the subject comprises extracting a nucleic acid sample from the sample from the subject, preferably an mRNA sample. In one embodiment, the expression level of the nucleic acid is determined by hybridizing the nucleic acid, or amplification products thereof, to a DNA microarray. Amplification products may be generated, for example, with reverse transcription, optionally followed by PCR amplification of the products.

(C) Genes Screened

In one embodiment, the predictive methods of the invention comprise determining the expression level of all the genes in the cluster that define at least one therapeutic sensitivity/resistance determinative metagene. In one embodiment, the predictive methods of the invention comprise determining the expression level of at least 50%, 60%, 70%, 80%, 90%, 95%, 98%, 99% of the genes in each of the clusters that defines 1, 2, 3, 4 or 5 or more therapeutic sensitivity/resistance determinative metagenes.

In one embodiment, at least 50%, 60%, 70%, 80%, 90%, 95%, 98%, 99% of the genes whose expression levels are determined to predict 5-FU sensitivity (or the genes in the cluster that define a metagene having said predictivity) are genes represented by the following symbols: ETS2, TP53BP1, ABCA2, COL1A2, SULT1A2, SULT1A1, SULT1A3, SULT1A4, HIST2H2AA, TPM3, SOX9, SERINC1, MTHFR, PKIG, CYP2A7P1, ZNF267, SNRPN, SNURF, GRIK5, PDE5A, BTF3, FAM49A, RNF139, HYPB, TPO, ZNF239, SYNPO, KIAA0895, HMGN3, LY6E, SMCP, ATP6V0A2, LOC388574, C1D, YT521, VIL2, POLE, OGDH, EIF5B, STX16, FLJ10534, THEM2, CDK2AP1, CREB3L1, IF127, B2M and CGREF1.

In one embodiment, at least 50%, 60%, 70%, 80%, 90%, 95%, 98%, 99% of the genes whose expression levels are determined to predict adriamycin sensitivity are genes represented by the following symbols: MLANA, PDGFA, ERCC4, RBBP4, ETS1, CDC6, BCL2, BCL2, BCL2, SKP1A, CDKN1B, DNM1, PMPCB, PBP, NEURL, CNOT4, APOF, NCK2, MGC33887, KIAA0934, SCARB2, TIA1, CLIC4, DAPK3, EIF4G3, ADAM 11, IL12A, AGTPBP1, EIF3S4, DKFZP564J0123, KCTD2, CPS1, SGCD, TAX1BP1, KPNA6, DPP6, ARFRP1, GORASP2, ALDH7A1, ID1, ZNF250, ACBD3, PLP2, HLA-DMA, PHF3, GLB1, KIAA0232, APOM, DGKZ, COL6A3, PPT2, EGFL8, SHC1, WARS, TRFP, CD53, C10orf26, PAK7, CLEC4M, ANGPT1, ANPEP, HAX1, UNC13B, OSBPL2, DDC, GNS, TUBA3, PKM2, RAD23B, LOC131185, KRT7, CNNM2, UGT2B7, ZFP95, HIPK3, HLA-DMB, SMA3, SMA5, UIP1, CASP1, CYP24A1 and IL1R.

In one embodiment, at least 50%, 60%, 70%, 80%, 90%, 95%, 98%, 99% of the genes whose expression levels are determined to predict cytoxan sensitivity (or the genes in the cluster that define a metagene having said predictivity) are genes represented by the following symbols: CYP2C19, PTPRO, EDNRB, MAP3K8, CCND2, BMP5, RPS6KB1, TRAV20, FCGRT, FN1, PPY, SCP2, CPSF1, UGT2B17, PDE3A, KCTD2, CCL19, MPST, RNPS1, SEC14L1, UROS, MTSS1, IGKC, LIMK2, MUC1, PML, LOC161527, UBTF, PRG2, CA2, TRPC4AP, PPP3R1, CSTF3, LOC400053, LOC57149 and NNT.

In one embodiment, at least 50%, 60%, 70%, 80%, 90%, 95%, 98%, 99% of the genes whose expression levels are determined to predict docetaxel sensitivity (or the genes in the cluster that define a metagene having said predictivity) are genes represented by the following symbols: ERCC4, BRF1, NCAM1, FARSLA, ERBB2, ERCC1, BAX, CTNNA1, FCGRT, FCGRT, NDUFS7, SLC22A5, SAFB2, C12orf22, KIAA0265, AK3L1, CLTB, FBL, BCL2L11, FLII, FOXD1, MRPS12, FLJ21168, RAB31, GAS7, SERINC1, RPS7, CORO2B, LRIG1, USP12, HLA-G, PLCB4, FANCC, GPR56, hfl-B5, BRD2, LOC253982, LY6H, RBMX2, MYL2, FLJ38348, ABCF3, TTC15, TUBA3, PCGF1, GJB3, INPP5A, PLLP, AQR and NF1.

In one embodiment, at least 50%, 60%, 70%, 80%, 90%, 95%, 98%, 99% of the genes whose expression levels are determined to predict etoposide sensitivity are genes represented by the following symbols: POLG, LIG3, IGFBP1, CYP2C9, VEGFC, EIF5, E2F4, ARG1, MAPT, ABCD2, FN1, IK, , KIAA0323, IKBKE, MRCL3, DAPK3, S100P, DKFZP564J0123, PAQR4, TXNDC, CA12, C9orf74, KPNA6, HYAL3, MKL1, RAMP1, DPP6, ACTR2, C2orf23, FCER1G, RBBP6, DPYD, RPA1, PDAP1, BTN3A2, ACTN1, RBMX, ELAC2, UGCG, SAPS2, CNNM2, PDPN, IRF5, CASP1, CREB5 and EPHB2.

In one embodiment, at least 50%, 60%, 70%, 80%, 90%, 95%, 98%, 99% of the genes whose expression levels are determined to predict paclitaxel sensitivity (or the genes in the cluster that define a metagene having said predictivity) are genes represented by the following symbols: PRKCB1, ERCC4, IGFBP3, ERBB2, PTPN11, ERCC1, , ERCC1, ATM, ROCK1, BCL2L11, HYPE, GATAD1, C6orf145, TFEC, GOLGA3, CDH19, CYP26A1, NUCB2, CCNF, ERCC1, EXT2, LMNA, PSMC5, POLE3, HMX1, RASSF7, LHX2, TUBA3, SEL1L, WDR67, ENO1, SNRPF, MAPT and PPP2CB.

In one embodiment, at least 50%, 60%, 70%, 80%, 90%, 95%, 98%, 99% of the genes whose expression levels are determined to predict sensitivity (or the genes in the cluster that define a metagene having said predictivity) are genes represented by the following symbols: BLR1, IL7, IGFBP1, PRKDC, PTPRD, ARHGEF16, UBC, PPP2R2B, MYCL1, MAP2K6, DUSP8, TOP2A, CDKN3, MYBL1, FARSLA, STMN1, MYC, ERCC1, TGFBR1, ABL1, MGMT, ITGB1, FGFR1, TGM2, CBX2, PCNT2, ADORA2A, EZH1, RPL15, CLPP, YWHAQ, VAMP5, RAB1A, BASP1, KBTBD2, MYO1C, KTN1, PDIA6, GLT8D1, C11orf9, SLC4A1, C1orf77, CAP2, SNF1LK, LRRC8B, TRAF2, GlyBP, CCL14, CCL15, ACSL3, ATF6, MYL6, , IGHM, RPS15A, S100P, HUWE1, PLS3, USP52, C16orf49, SPAM1, EIF4EBP2, C9orf74, ILK, UCKL1, LEREPO4, NCOA1, APLP1, ARHGEF4, SLC25A17, H2AFY, ANXA11, DHCR24, LILRB5, TPM1, TPM1, SPN, KIAA0485, CD163, MRPL49, LMNB2, C9orf10, TTC1, MYH11, SLC27A2, RASSF2, METAP2, ASGR2, CSPG2, MDK, KCNMB1, ZNF193, KIAA0247, NDUFS1, G1P2, ACTN2, RPA1, STAB1, LASS6, HDAC1, STX7, UBADC1, CHEK1, CCR4, RALA, CACNA1D, ATP6V0A1, TUBB-PARALOG, ACADS, MAN1A1, SEPW1, USP22, IGSF4C, FCMD, ACO1, CA2, M6PRBP1, C6orf162, C1S, , PRKCA, BTAF1, ZNF274, CTBP2, MGC11308, KPNB1, STAT6, ATF4, TMAP1, KRT7, TNFRSF17, KCNJ13, AFF3, HSPA12A, SRRM1, OPTN, OPTN, PDPN, EWSR1, IFI35, NR4A2, HIST1H1E, AVPR1B, SPARC, THBS1, CCL2, PIM1, ITGA3 and ITGB8.

Table 5 shows the genes in the cluster that define metagenes 1-7 and indicates the therapeutic agent whose sensitivity it predicts. In one embodiment, at least 3, 5, 7, 9, 10, 12, 14, 16, 18, 20, 25, 30, 40 or 50 genes in the cluster of genes defining a metagene used in the methods described herein are common to metagene 1, 2, 3, 4, 5, 6 or 7, or to combinations thereof.

(D) Metagene Valuation

In one embodiment, the predictive methods of the invention comprise defining the value of one or more metagenes from the expression levels of the genes. A metagene value is defined by extracting a single dominant value from a cluster of genes associated with sensitivity to an anti-cancer agent, preferably an anti-cancer agent such as docetaxel, paclitaxel, topotecan, adriamycin, etoposide, fluorouracil (5-FU), and cyclophosphamide. In one embodiment, the agent is selected from alkylating agents (e.g., nitrogen mustards), antimetabolites (e.g., pyrimidine analogs), radioactive isotopes (e.g., phosphorous and iodine), miscellaneous agents (e.g., substituted ureas) and natural products (e.g., vinca alkyloids and antibiotics). In another embodiment, the therapeutic agent is selected from the group consisting of allopurinol sodium, dolasetron mesylate, pamidronate disodium, etidronate, fluconazole, epoetin alfa, levamisole HCL, amifostine, granisetron HCL, leucovorin calcium, sargramostim, dronabinol, mesna, filgrastim, pilocarpine HCL, octreotide acetate, dexrazoxane, ondansetron HCL, ondansetron, busulfan, carboplatin, cisplatin, thiotepa, melphalan HCL, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, plimycin, mitomycin, pentostatin, mitoxantrone, valrubicin, cytarabine, fludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alpha-2b, mitotane, procarbazine HCL, vinorelbine tartrate, E. coli L-asparaginase, Erwinia L-asparaginase, vincristine sulfate, denileukin diftitox, aldesleukin, rituximab, interferon alpha-2a, paclitaxel, docetaxel, BCG live (intravesical), vinblastine sulfate, etoposide, tretinoin, teniposide, porfimer sodium, fluorouracil, betamethasone sodium phosphate and betamethasone acetate, letrozole, etoposide citrororum factor, folinic acid, calcium leucouorin, 5-fluorouricil, adriamycin, cytoxan, and diamino-dichloro-platinum.

In a preferred embodiment, the dominant single value is obtained using single value decomposition (SVD). In one embodiment, the cluster of genes of each metagene or at least of one metagene comprises at least 3, 4, 5, 6, 7, 8, 9, 10, 12, 15, 18, 20 or 25 genes. In one embodiment, the predictive methods of the invention comprise defining the value of 2, 3, 4, 5, 6, 7, 8, 9 or 10 or more metagenes from the expression levels of the genes.

In preferred embodiments of the methods described herein, at least 1, 2, 3, 4, 5, 6, 7, 8 or 9 of the metagenes is metagene 1, 2, 3, 4, 5, 6, or 7. In one embodiment, at least one of the metagenes comprises 3, 4, 5, 6, 7, 8, 9 or 10 or more genes in common with any one of metagenes 1, 2, 3, 4, 5, 6, or 7. In one embodiment, a metagene shares at least 50%, 60%, 70%, 80%, 90%, 95%, 98%, 99% of the genes in its cluster in common with a metagene selected from 1, 2, 3, 4, 5, 6, or 7.

In one embodiment, the predictive methods of the invention comprise defining the value of 2, 3, 4, 5, 6, 7, 8 or more metagenes from the expression levels of the genes. In one embodiment, the cluster of genes from which any one metagene is defined comprises at least 3, 4, 5, 6, 7, 8, 9, 10, 11, 12, 13, 14, 15, 16, 17, 18, 19, 20, 22 or25 genes.

In one embodiment, the predictive methods of the invention comprise defining the value of at least one metagene wherein the genes in the cluster of genes from which the metagene is defined, shares at least 50%, 60%, 70%, 80%, 90%, 95% or 98% of genes in common to any one of metagenes 1, 2, 3, 4, 5, 6, or 7. In one embodiment, the predictive methods of the invention comprise defining the value of at least two metagenes, wherein the genes in the cluster of genes from which each metagene is defined share at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of at least three metagenes, wherein the genes in the cluster of genes from which each metagene is defined shares at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of at least four metagenes, wherein the genes in the cluster of genes from which each metagene is defined shares at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of at least five metagenes, wherein the genes in the cluster of genes from which each metagene is defined shares at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of a metagene from a cluster of genes, wherein at least 2, 3, 4, 5, 6, 7, 8, 9, 10, 11, 12, 13, 14, 15, 16, 17, 18, 19 or 20 genes in the cluster are selected from the genes listed in Table 5.

In one embodiment, at least one of the metagenes is metagene 1, 2, 3, 4, 5, 6, or 7. In one embodiment, at least two of the metagenes are selected from metagenes 1, 2, 3, 4, 5, 6, or 7. In one embodiment, at least three of the metagenes are selected from metagenes 1, 2, 3, 4, 5, 6, or 7. In one embodiment, at least three of the metagenes are selected from metagenes 1, 2, 3, 4, 5, 6, or 7. In one embodiment, at least four of the metagenes are selected from metagenes 1, 2, 3, 4, 5, 6, or 7. In one embodiment, at least five or more of the metagenes are selected from metagenes 1, 2, 3, 4, 5, 6, or 7. In one embodiment of the methods described herein, one of the metagenes whose value is defined (i) is metagene I or (ii) shares at least 2, 3, 4, 5, 6, 7, 8, 9, 10, 11, 12 or 13 genes in common with metagene 1. In one embodiment of the methods described herein, one of the metagenes whose value is defined (i) is metagene 2 or (ii) shares at least 2, 3, 4, 5, 6, 7, 8, 9, 10, 11 or 12 genes in common with metagene 2. In one embodiment of the methods described herein, one of the metagenes whose value is defined (i) is metagene 3 or (ii) shares at least 2, 3 or 4 genes in common with metagene 3. In one embodiment of the methods described herein, one of the metagenes whose value is defined (i) is metagene 4 or (ii) shares at least 2, 3, 4, 5, 6, 7, 8, 9, 10, 11, 12, 13, 14, 15, 16, 17, 18, 19, 20, 21, 22, 23, 24 or 25 genes in common with metagene 4. In one embodiment of the methods described herein, one of the metagenes whose value is defined (i) is metagene 5 or (ii) shares at least 2, 3, 4, 5, 6, 7, 8, 9, 10, 11, 12, 13, 14 or 15 genes in common with metagene 5. In one embodiment of the methods described herein, one of the metagenes whose value is defined (i) is metagene 6 or (ii) shares at least 2, 3, 4, 5, 6, 7, 8, 9, 10, 11, 12 or 13 genes in common with metagene 6. In one embodiment of the methods described herein, one of the metagenes whose value is defined (i) is metagene 7 or (ii) shares at least 2, 3, 4, 5, 6, 7, 8, 9 or 10 genes in common with metagene 7.

In one embodiment, the clusters of genes that define each metagene are identified using supervised classification methods of analysis previously described. See, for example, West, M. et al. Proc Natl Acad Sci USA 98, 11462-11467 (2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models, such as binary regression models, assign the relative probability of sensitivity to an anti-cancer agent.

(E) Predictions from Tree Models

In one embodiment, the predictive methods of the invention comprise averaging the predictions of one or more statistical tree models applied to the metagenes values, wherein each model includes one or more nodes, each node representing a metagene, each node including a statistical predictive probability of sensitivity to an anti-cancer agent. The statistical tree models may be generated using the methods described herein for the generation of tree models. General methods of generating tree models may also be found in the art (See for example Pitman et al., Biostatistics 2004;5:587-601; Denison et al. Biometrika 1999;85:363-77; Nevins et al. Hum Mol Genet 2003;12:R153-7; Huang et al. Lancet 2003;361 :1590-6; West et al. Proc Natl Acad Sci USA 2001;98:11462-7; U.S. Patent Pub. Nos. 2003-0224383; 2004-0083084; 2005-0170528; 2004-0106113; and U.S. application Ser. No. 11/198782).

In one embodiment, the predictive methods of the invention comprise deriving a prediction from a single statistical tree model, wherein the model includes one or more nodes, each node representing a metagene, each node including a statistical predictive probability of sensitivity to an anti-cancer agent. In a preferred embodiment, the tree comprises at least 2 nodes. In a preferred embodiment, the tree comprises at least 3 nodes. In a preferred embodiment, the tree comprises at least 3 nodes. In a preferred embodiment, the tree comprises at least 4 nodes. In a preferred embodiment, the tree comprises at least 5 nodes.

In one embodiment, the predictive methods of the invention comprise averaging the predictions of one or more statistical tree models applied to the metagenes values, wherein each model includes one or more nodes, each node representing a metagene, each node including a statistical predictive probability of sensitivity to an anti-cancer agent. Accordingly, the invention provides methods that use mixed trees, where a tree may contain at least two nodes, where each node represents a metagene representative to the sensitivity/resistance to a particular agent.

In one embodiment, the statistical predictive probability is derived from a Bayesian analysis. In another embodiment, the Bayesian analysis includes a sequence of Bayes factor based tests of association to rank and select predictors that define a node binary split, the binary split including a predictor/threshold pair. Bayesian analysis is an approach to statistical analysis that is based on the Bayes law, which states that the posterior probability of a parameter p is proportional to the prior probability of parameter p multiplied by the likelihood of p derived from the data collected. This methodology represents an alternative to the traditional (or frequentist probability) approach: whereas the latter attempts to establish confidence intervals around parameters, and/or falsify a-priori null-hypotheses, the Bayesian approach attempts to keep track of how apriori expectations about some phenomenon of interest can be refined, and how observed data can be integrated with such a-priori beliefs, to arrive at updated posterior expectations about the phenomenon. Bayesian analysis have been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” nodes using a splitting rule. For instance, for clarification, training data contains sample vectors that have one or more measurement variables and one variable that determines that class of the sample. Various splitting rules may be used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently, used purity functions are entropy, gini-index, and towing rule. A statistical predictive tree model to which Bayesian analysis is applied may consistently deliver accurate results with high predictive capabilities.

Gene expression signatures that reflect the activity of a given pathway may be identified using supervised classification methods of analysis previously described (e.g., West, M. et al. Proc Natl Acad Sci USA 98, 11462-11467, 2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models assign the relative probability of sensitivity to an anti-cancer agent.

One aspect of the invention provides methods for defining one or more statistical tree models predictive of lung sensitivity to an anti-cancer agent. In one embodiment, the methods for defining one or more statistical tree models predictive of cancer sensitivity to an anti-cancer agent comprise determining the expression level of multiple genes in a set of cancer samples. The samples include samples from subjects with cancer and samples from subjects without cancer. In one embodiment, at least 5, 10, 15, 20, 25, 30, 35, 40, 45, 50, 60, 70, 80, 90 or 100 samples from each of the two classes are used. The expression level of genes may be determined using any of the methods described in the preceding sections or any know in the art.

In one embodiment, the methods for defining one or more statistical tree models predictive of cancer sensitivity to an anti-cancer agent comprise identifying clusters of genes associated with metastasis by applying correlation-based clustering to the expression level of the genes. In one embodiment, the clusters of genes that define each metagene are identified using supervised classification methods of analysis previously described. See, for example, West, M. et al. Proc Natl Acad Sci USA 98, 11462-11467 (2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models assign the relative probability of sensitivity to an anti-cancer agent.

In one embodiment, identification of the clusters comprises screening genes to reduce the number by eliminating genes that show limited variation across samples or that are evidently expressed at low levels that are not detectable at the resolution of the gene expression technology used to measure levels. This removes noise and reduces the dimension of the predictor variable. In one embodiment, identification of the clusters comprises clustering the genes using k-means, correlated-based clustering. Any standard statistical package may be used, such as the xcluster software created by Gavin Sherlock (http://genetics.stanford.edu/˜sherlock/cluster.html). A large number of clusters may be targeted so as to capture multiple, correlated patterns of variation across samples, and generally small numbers of genes within clusters. In one embodiment, identification of the clusters comprises extracting the dominant singular factor (principal component) from each of the resulting clusters. Again, any standard statistical or numerical software package may be used for this; this analysis uses the efficient, reduced singular value decomposition function. In one embodiment, the foregoing methods comprise defining one or more metagenes, wherein each metagene is defined by extracting a single dominant value using single value decomposition (SVD) from a cluster of genes associated with estimating the efficacy of a therapeutic agent in treating a subject afflicted with cancer.

In one embodiment, the methods for defining one or more statistical tree models predictive of cancer sensitivity to an anti-cancer agent comprise defining a statistical tree model, wherein the model includes one or more nodes, each node representing a metagene, each node including a statistical predictive probability of the efficacy of a therapeutic agent in treating a subject afflicted with cancer. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. Iterative out-of-sample, cross-validation predictions are then performed leaving each tumor out of the data set one at a time, refitting the model from the remaining tumors and using it to predict the hold-out case. This rigorously tests the predictive value of a model and mirrors the real-world prognostic context where prediction of new cases as they arise is the major goal.

In one embodiment, a formal Bayes' factor measure of association may be used in the generation of trees in a forward-selection process as implemented in traditional classification tree approaches. Consider a single tree and the data in a node that is a candidate for a binary split. Given the data in this node, one may construct a binary split based on a chosen (predictor, threshold) pair (χ, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 prior, Bayes' factors of 2.2 ,2.9, 3.7 and 5.3 correspond, approximately, to probabilities of 0.9, 0.95, 0.99 and 0.995, respectively. This guides the choice of threshold, which may be specified as a single value for each level of the tree. Bayes' factor thresholds of around 3 in a range of analyses may be used. Higher thresholds limit the growth of trees by ensuring a more stringent test for splits.

In one non-limiting exemplary embodiment of generating statistical tree models, prior to statistical modeling, gene expression data is filtered to exclude probe sets with signals present at background noise levels, and for probe sets that do not vary significantly across tumor samples. A metagene represents a group of genes that together exhibit a consistent pattern of expression in relation to an observable phenotype. Each signature summarizes its constituent genes as a single expression profile, and is here derived as the first principal component of that set of genes (the factor corresponding to the largest singular value) as determined by a singular value decomposition. Given a training set of expression vectors (of values across metagenes) representing two biological states, a binary probit regression model may be estimated using Bayesian methods. Applied to a separate validation data set, this leads to evaluations of predictive probabilities of each of the two states for each case in the validation set. When predicting sensitivity to an anti-cancer agent from an Tumor sample, gene selection and identification is based on the training data, and then metagene values are computed using the principal components of the training data and additional expression data. Bayesian fitting of binary probit regression models to the training data then permits an assessment of the relevance of the metagene signatures in within-sample classification, and estimation and uncertainty assessments for the binary regression weights mapping metagenes to probabilities of relative pathway status. Predictions of sensitivity to an anti-cancer agent are then evaluated, producing estimated relative probabilities—and associated measures of uncertainty—of sensitivity to an anti-cancer agent across the validation samples. Hierarchical clustering of sensitivity to anti-cancer agent predictions may be performed using Gene Cluster 3.0 testing the null hypothesis, which is that the survival curves are identical in the overall population.

In one embodiment, the each statistical tree model generated by the methods described herein comprises 2, 3, 4, 5, 6 or more nodes. In one embodiment of the methods described herein for defining a statistical tree model predictive of sensitivity/resistance to a therapeutic, the resulting model predicts cancer sensitivity to an anti-cancer agent with at least 70%, 80%, 85%, or 90% or higher accuracy. In another embodiment, the model predicts sensitivity to an anti-cancer agent with greater accuracy than clinical variables. In one embodiment, the clinical variables are selected from age of the subject, gender of the subject, tumor size of the sample, stage of cancer disease, histological subtype of the sample and smoking history of the subject. In one embodiment, the cluster of genes that define each metagene comprise at least 3, 4, 5, 6, 7, 8, 9, 10, 12 or 15 genes. In one embodiment, the correlation-based clustering is Markov chain correlation-based clustering or K-means clustering.

Diagnostic Business Methods

One aspect of the invention provides methods of conducting a diagnostic business, including a business that provides a health care practitioner with diagnostic information for the treatment of a subject afflicted with cancer. One such method comprises one, more than one, or all of the following steps: (i) obtaining an tumor sample from the subject; (ii) determining the expression level of multiple genes in the sample; (iii) defining the value of one or more metagenes from the expression levels of step (ii), wherein each metagene is defined by extracting a single dominant value using single value decomposition (SVD) from a cluster of genes associated with sensitivity to an anti-cancer agent; (iv) averaging the predictions of one or more statistical tree models applied to the values, wherein each model includes one or more nodes, each node representing a metagene, each node including a statistical predictive probability of sensitivity to an anti-cancer agent; and (v) providing the health care practitioner with the prediction from step (iv).

In one embodiment, obtaining a tumor sample from the subject is effected by having an agent of the business (or a subsidiary of the business) remove a tumor sample from the subject, such as by a surgical procedure. In another embodiment, obtaining a tumor sample from the subject comprises receiving a sample from a health care practitioner, such as by shipping the sample, preferably frozen. In one embodiment, the sample is a cellular sample, such as a mass of tissue. In one embodiment, the sample comprises a nucleic acid sample, such as a DNA, cDNA, mRNA sample, or combinations thereof, which was derived from a cellular tumor sample from the subject. In one embodiment, the prediction from step (iv) is provided to a health care practitioner, to the patient, or to any other business entity that has contracted with the subject.

In one embodiment, the method comprises billing the subject, the subject's insurance carrier, the health care practitioner, or an employer of the health care practitioner. A government agency, whether local, state or federal, may also be billed for the services. Multiple parties may also be billed for the service.

In some embodiments, all the steps in the method are carried out in the same general location. In certain embodiments, one or more steps of the methods for conducting a diagnostic business are performed in different locations. In one embodiment, step (ii) is performed in a first location, and step (iv) is performed in a second location, wherein the first location is remote to the second location. The other steps may be performed at either the first or second location, or in other locations. In one embodiment, the first location is remote to the second location. A remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. In one embodiment, two locations that are remote relative to each other are at least 1, 2, 3, 4, 5, 10, 20, 50, 100, 200, 500, 1000, 2000 or 5000 km apart. In another embodiment, the two locations are in different countries, where one of the two countries is the United States.

Some specific embodiments of the methods described herein where steps are performed in two or more locations comprise one or more steps of communicating information between the two locations. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In one specific embodiment, the method comprises one or more data transmission steps between the locations. In one embodiment, the data transmission step occurs via an electronic communication link, such as the internet. In one embodiment, the data transmission step from the first to the second location comprises experimental parameter data, such as the level of gene expression of multiple genes. In some embodiments, the data transmission step from the second location to the first location comprises data transmission to intermediate locations. In one specific embodiment, the method comprises one or more data transmission substeps from the second location to one or more intermediate locations and one or more data transmission substeps from one or more intermediate locations to the first location, wherein the intermediate locations are remote to both the first and second locations. In another embodiment, the method comprises a data transmission step in which a result from gene expression is transmitted from the second location to the first location.

In one embodiment, the methods of conducting a diagnostic business comprise the step of determining if the subject carries an allelic form of a gene whose presence correlates to sensitivity or resistance to a chemotherapeutic agent. This may be achieved by analyzing a nucleic acid sample from the patient and determining the DNA sequence of the allele. Any technique known in the art for determining the presence of mutations or polymorphisms may be used. The method is not limited to any particular mutation or to any particular allele or gene. For example, mutations in the epidermal growth factor receptor (EGFR) gene are found in human lung adenocarcinomas and are associated with sensitivity to the tyrosine kinase inhibitors gefitinib and erlotinib. (See, e.g., Yi et al. Proc Natl Acad Sci USA. 2006 May 16;103(20):7817-22; Shimato et al. Neuro-oncol. 2006 April;8(2):137-44). Similarly, mutations in breast cancer resistance protein (BCRP) modulate the resistance of cancer cells to BCRP-substrate anticancer agents (Yanase et al., Cancer Lett. 2006 Mar. 8;234(1):73-80).

Arrays and Gene Chips and Kits Comprising thereof

Arrays and microarrays which contain the gene expression profiles for determining responsivity to platinum-based therapy and/or responsivity to salvage agents are also encompassed within the scope of this invention. Methods of making arrays are well-known in the art and as such, do not need to be described in detail here.

Such arrays can contain the profiles of at least 5, 10, 15, 25, 50, 75, 100, 150, or 200 genes as disclosed in the Tables. Accordingly, arrays for detection of responsivity to particular therapeutic agents can be customized for diagnosis or treatment of ovarian cancer. The array can be packaged as part of kit comprising the customized array itself and a set of instructions for how to use the array to determine an individual's responsivity to a specific cancer therapeutic agent.

Also provided are reagents and kits thereof for practicing one or more of the above described methods. The subject reagents and kits thereof may vary greatly. Reagents of interest include reagents specifically designed for use in production of the above described metagene values.

One type of such reagent is an array probe of nucleic acids, such as a DNA chip, in which the genes defining the metagenes in the therapeutic efficacy predictive tree models are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373203; and EP 785280.

The DNA chip is convenient to compare the expression levels of a number of genes at the same time. DNA chip-based expression profiling can be carried out, for example, by the method as disclosed in “Microarray Biochip Technology” (Mark Schena, Eaton Publishing, 2000). A DNA chip comprises immobilized high-density probes to detect a number of genes. Thus, the expression levels of many genes can be estimated at the same time by a single-round analysis. Namely, the expression profile of a specimen can be determined with a DNA chip. A DNA chip may comprise probes, which have been spotted thereon, to detect the expression level of the metagene-defining genes of the present invention. A probe may be designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. A DNA chip that is obtained by the method as described above can be used estimating the efficacy of a therapeutic agent in treating a subject afflicted with cancer according to the present invention.

DNA microarray and methods of analyzing data from microarrays are well-described in the art, including in DNA Microarrays: A Molecular Cloning Manual, Ed. by Bowtel and Sambrook (Cold Spring Harbor Laboratory Press, 2002); Microarrays for an Integrative Genomics by Kohana (MIT Press, 2002); A Biologist's Guide to Analysis ofDNA Microarray Data, by Knudsen (Wiley, John & Sons, Incorporated, 2002); DNA Microarrays: A Practical Approach, Vol. 205 by Schema (Oxford University Press, 1999); and Methods of Microarray Data Analysis II, ed. by Lin et al. (Kluwer Academic Publishers, 2002).

One aspect of the invention provides a gene chip having a plurality of different oligonucleotides attached to a first surface of the solid support and having specificity for a plurality of genes, wherein at least 50% of the genes are common to those of metagenes 1, 2, 3, 4, 5, 6 and/or 7. In one embodiment, at least 70%, 80%, 90% or 95% of the genes in the gene chip are common to those of metagenes 1, 2, 3, 4, 5, 6 and/or 7.

One aspect of the invention provides a kit comprising: (a) any of the gene chips described herein; and (b) one of the computer-readable mediums described herein.

In some embodiments, the arrays include probes for at least 2, 3, 4, 5, 6, 7, 8, 9, 10, 15, 20, 25, 30, 40, or 50 of the genes listed in Table 5. In certain embodiments, the number of genes that are from table 4 that are represented on the array is at least 5, at least 10, at least 25, at least 50, at least 75 or more, including all of the genes listed in the table. Where the subject arrays include probes for additional genes not listed in the tables, in certain embodiments the number % of additional genes that are represented does not exceed about 50%, 40%, 30%, 20%, 15%, 10%, 8%, 6%, 5%, 4%, 3%, 2% or 1%. In some embodiments, a great majority of genes in the collection are genes that define the metagenes of the invention, where by great majority is meant at least about 75%, usually at least about 80% and sometimes at least about 85, 90, 95% or higher, including embodiments where 100% of the genes in the collection are metagene-defining genes.

The kits of the subject invention may include the above described arrays. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

Computer Readable Media Comprising Gene Expression Profiles

The invention also contemplates computer readable media that comprises gene expression profiles. Such media can contain all of part of the gene expression profiles of the genes listed in the Tables. The media can be a list of the genes or contain the raw data for running a user's own statistical calculation, such as the methods disclosed herein.

Program Products/Systems

Another aspect of the invention provides a program product (i.e., software product) for use in a computer device that executes program instructions recorded in a computer-readable medium to perform one or more steps of the methods described herein, such for estimating the efficacy of a therapeutic agent in treating a subject afflicted with cancer.

On aspect of the invention provides a computer readable medium having computer readable program codes embodied therein, the computer readable medium program codes performing one or more of the following fuictions: defining the value of one or more metagenes from the expression levels genes; defining a metagene value by extracting a single dominant value using singular value decomposition (SVD) from a cluster of genes associated tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

Another related aspect of the invention provides kits comprising the program product or the computer readable medium, optionally with a computer system. On aspect of the invention provides a system, the system comprising: a computer; a computer readable medium, operatively coupled to the computer, the computer readable medium program codes performing one or more of the following functions: defining the value of one or more metagenes from the expression levels genes; defining a metagene value by extracting a single dominant value using singular value decomposition (SVD) from a cluster of genes associated tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

In one embodiment, the program product comprises: a recordable medium; and a plurality of computer-readable instructions executable by the computer device to analyze data from the array hybridization steps, to transmit array hybridization from one location to another, or to evaluate genome-wide location data between two or more genomes. Computer readable media include, but are not limited to, CD-ROM disks (CD-R, CD-RW), DVD-RAM disks, DVD-RW disks, floppy disks and magnetic tape.

A related aspect of the invention provides kits comprising the program products described herein. The kits may also optionally contain paper and/or computer-readable format instructions and/or information, such as, but not limited to, information on DNA microarrays, on tutorials, on experimental procedures, on reagents, on related products, on available experimental data, on using kits, on chemotherapeutic agents including there toxicity, and on other information. The kits optionally also contain in paper and/or computer-readable format information on minimum hardware requirements and instructions for running and/or installing the software. The kits optionally also include, in a paper and/or computer readable format, information on the manufacturers, warranty information, availability of additional software, technical services information, and purchasing information. The kits optionally include a video or other viewable medium or a link to a viewable format on the internet or a network that depicts the use of the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of data, as well as the transmission of data steps, can be implemented by the use of one or more computer systems. Computer systems are readily available. The processing that provides the displaying and analysis of image data for example, can be performed on multiple computers or can be performed by a single, integrated computer or any variation thereof. For example, each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM).

Each computer typically includes a program product reader that accepts a program product storage device from which the program product reader can read data (and to which it can optionally write data). The program product reader can include, for example, a disk drive, and the program product storage device can include a removable storage medium such as, for example, a magnetic floppy disk, an optical CD-ROM disc, a CD-R disc, a CD-RW disc and a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods understood by those skilled in the art. The computer that implements the client side processing, and the computer that implements the server side processing or any other computer device of the system, can include any conventional computer suitable for implementing the functionality described herein.

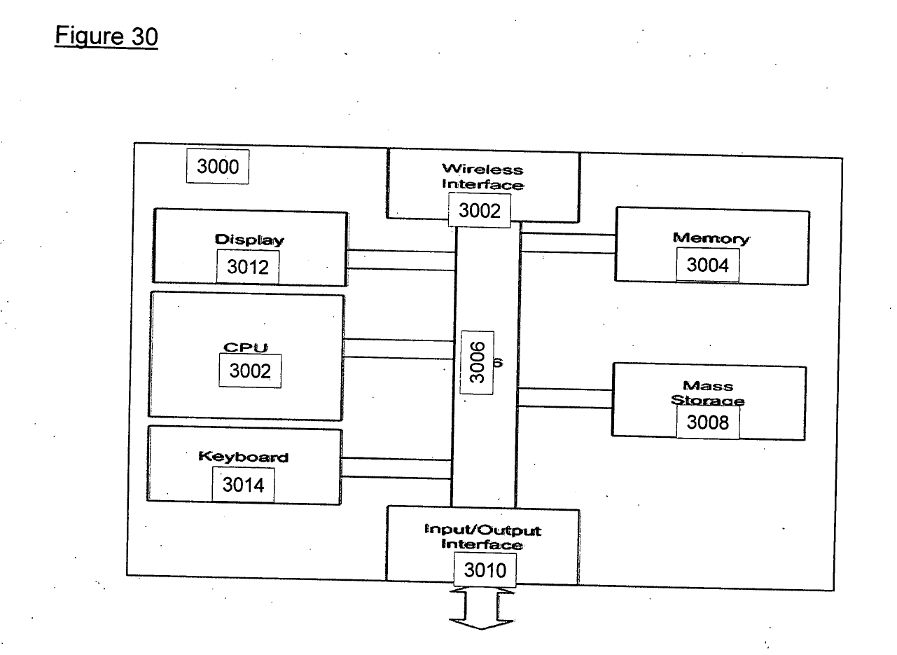

FIG. 30 shows a functional block diagram of general purpose computer system 3000 for performing the functions of the software according to an illustrative embodiment of the invention. The exemplary computer system 3000 includes a central processing unit (CPU) 3002, a memory 33004, and an interconnect bus 3006. The CPU 3002 may include a single microprocessor or a plurality of microprocessors for configuring computer system 3000 as a multi-processor system. The memory 3004 illustratively includes a main memory and a read only memory. The computer 3000 also includes the mass storage device 3008 having, for example, various disk drives, tape drives, etc. The main memory 3004 also includes dynamic random access memory (DRAM) and high-speed cache memory. In operation, the main memory 3004 stores at least portions of instructions and data for execution by the CPU 3002.

The mass storage 3008 may include one or more magnetic disk or tape drives or optical disk drives, for storing data and instructions for use by the CPU 3002. At least one component of the mass storage system 3008, preferably in the form of a disk drive or tape drive, stores one or more databases, such as databases containing of transcriptional start sites, genomic sequence, promoter regions, or other information.

The mass storage system 3008 may also include one or more drives for various portable media, such as a floppy disk, a compact disc read only memory (CD-ROM), or an integrated circuit non-volatile memory adapter (i.e., PC-MCIA adapter) to input and output data and code to and from the computer system 3000.

The computer system 3000 may also include one or more input/output interfaces for communications, shown by way of example, as interface 3010 for data communications via a network. The data interface 3010 may be a modem, an Ethernet card or any other suitable data communications device. To provide the functions of a computer system according to FIG. 30 the data interface 3010 may provide a relatively high-speed link to a network, such as an intranet, internet, or the Internet, either directly or through an another external interface. The communication link to the network may be, for example, optical, wired, or wireless (e.g., via satellite or cellular network). Alternatively, the computer system 3000 may include a mainframe or other type of host computer system capable of Web-based communications via the network.

The computer system 3000 also includes suitable input/output ports or use the interconnect bus 3006 for interconnection with a local display 3012 and keyboard 3014 or the like serving as a local user interface for programming and/or data retrieval purposes. Alternatively, server operations personnel may interact with the system 3000 for controlling and/or programming the system from remote terminal devices via the network.

The computer system 3000 may run a variety of application programs and stores associated data in a database of mass storage system 3008. One or more such applications may enable the receipt and delivery of messages to enable operation as a server, for implementing server functions relating to obtaining a set of nucleotide array probes tiling the promoter region of a gene or set of genes.

The components contained in the computer system 3000 are those typically found in general purpose computer systems used as servers, workstations, personal computers, network terminals, and the like. In fact, these components are intended to represent a broad category of such computer components that are well known in the art.

It will be apparent to those of ordinary skill in the art that methods involved in the present invention may be embodied in a computer program product that includes a computer usable and/or readable medium. For example, such a computer usable medium may consist of a read only memory device, such as a CD ROM disk or conventional ROM devices, or a random access memory, such as a hard drive device or a computer diskette, having a computer readable program code stored thereon.

The following examples are provided to illustrate aspects of the invention but are not intended to limit the invention in any manner.

### EXAMPLES

### Example 1

### Use of Platinum Chemotherapy Responsivity Predictor Set and Salvage Therapy Resonsivitiy Predictor Set

The purpose of this study was to develop an integrated genomic-based approach to personalized treatment of patients with advanced-stage ovarian cancer. The inventors have utilized gene expression profiles to identify patients likely to be resistant to primary platinum-based chemotherapy and also to identify alternate targeted therapeutic options for patients with de-novo platinum resistant disease.

Material and Methods

Patients and tissue samples—Clinicopathologic characteristics of 119 ovarian cancer samples included in this study are detailed in Table 1. All ovarian cancers were obtained at initial cytoreductive surgery from patients treated at Duke University Medical Center and H. Lee Moffitt Cancer Center & Research Institute, who then received platinum-based primary chemotherapy. The samples were divided (70/30 ratio) into training and validation sets. As a result, 83/119 (70%) samples were randomly selected for the training set, and 36/119 (30%) samples selected for the validation set. In the training set a total of 59/83 (71%) patients demonstrated a complete response (CR)—and 24/83 (29%) patients demonstrated an incomplete response (IR) to primary platinum-based therapy following surgery. In the validation set a total of 26/36 (72%) patients demonstrated a complete response (CR)—and 10/36 (28%) patients demonstrated an incomplete response (IR) to primary platinum-based therapy. The distribution of CR and IR in both training and validation sets was selected to reflect clinical complete response rates of approximately 70%. The distribution of debulking status within the training and validation sets was equally balanced. All tissues were collected under the auspices of respective IRB approved protocol with written informed consent.

Measurement of clinical response—Response to therapy in ovarian cancer patients was evaluated from the medical record using standard WHO criteria for patients with measurable disease.²⁸ CA-125 was used to classify responses only in the absence of a measurable lesion; CA-125 response criteria was based on established guidelines.^{29,30} A complete response (CR) was defined as a complete disappearance of all measurable and assessable disease or, in the absence of measurable lesions, a normalization of the CA-125 level following adjuvant therapy. An incomplete response (IR) included patients who demonstrated only a partial response (PR), had stable disease (SD), or demonstrated progressive disease (PD) during primary therapy. A partial response was considered a 50% or greater reduction in the product obtained from measurement of each bi-dimensional lesion for at least 4 weeks or a drop in the CA-125 by at least 50% for at least 4 weeks. Disease progression was defined as a 50% or greater increase in the product from any lesion documented within 8 weeks of initiation of therapy, the appearance of any new lesion within 8 weeks of initiation of therapy, or any increase in the CA-125 from baseline at initiation of therapy. Stable disease was defined as disease not meeting any of the above criteria.

RNA and microarray analysis—Frozen tissue samples were embedded in OCT medium, sections were cut and slide-mounted. Slides were stained with hematoxylin and eosin to assure that samples included greater than 70% tumor content. Approximately 30 mg of tissue was used for RNA isolation. Approximately 30 mg of tissue was added to a chilled BioPulverizer H tube (Bio101). Lysis buffer from the Qiagen RNeasy Mini kit was added and the tissue homogenized for 20 seconds in a Mini-Beadbeater (Biospec Products). Tubes were spun briefly to pellet the garnet mixture and reduce foam. The lysate was passaged through a 21 gauge needle 10 times to shear genomic DNA. Total RNA was extracted using the Qiagen RNeasy Mini kit. Quality of the RNA was measured using an Agilent 2100 Bioanalzyer. Affymetrix DNA microarray analysis was prepared according to the manufacturer's instructions and targets were hybridized to the Human U133A GeneChip.

Statistical analysis—The expression intensities for all genes across the samples were normalized using RMA,³¹ including probe-level quantile normalization and background correction, as implemented in the Bioconductor software suite.32 RMA data was prescreened to remove genes/probes with trivial variation across the sample and low median expression levels, thus 6088 genes/probes were used in the analysis. The remaining RMA data was further processed by applying sparse regression model methods,³³ to correct for assay artifacts, the resulting expression files are available at http://data.cgt.duke.edu/platinum.php.

A binary logistic regression model analysis and a stochastic regression model search, called Shotgun Stochastic Search (SSS), was used to determine platinum response predictions models in the training set of 83 samples. The predictive analysis evaluated regression models linking log values of observed expression levels of small numbers of genes to platinum response and debulking status. As mentioned in previous publications,^{34,35} the challenge of statistical analysis is to search for subsets of genes that together define significant predictive regressions—that is, to select both the number k of genes, or variables (platinum response and debulking status), and then the specific set of genes {x₁, . . . , xₖ} by searching over subsets. This includes the possibility of no association with any genes, i.e., k=0. Technically, with many genes available this requires some form of stochastic search, i.e., shotgun stochastic search (that, in a distributed computer environment, allows the rapid evaluation of many such models so long as the search is constrained to values of k that are reasonably small, a precept consistent with both the small sample size constraint of many gene expression studies and also scientific parsimony and the need to penalize models on larger numbers of predictors to avoid over-fitting).

With several thousand genes as possible predictors (subsets of the 6088 genes/probes), there is a large number of candidate regressions to explore even when restricting the number of genes in any one model to be no more than eight genes. The parallel computational strategies implemented are very efficient and the search over models generally focuses quickly on subsets of relevant models with higher probability (if such exist). In this analysis with the training set n=83 samples, the average of 5000 small models (total number of genes=1727), confirms that a number of models containing 1-5 genes are of some interest. The Bayesian analysis heavily penalizes more complex models, initially very strongly favoring the null hypothesis of no significant predictors in this model context among the thousands of genes in a manner that naturally counters the false discovery propensity of purely likelihood-based model search analyses. In addition, routine calculations confirm that the false-positive rate for discovery of single variable regressions as significant as those identified among the top candidates here is small. From the 5000 regression models that identify a total of 1727 genes, Table 2 lists the 100 genes that contribute the most weight in the prediction and that appeared most often within the models. The full list of 1727 genes is posted on the web site mentioned earlier. The overall practical relevance of the set of regressions identified (as opposed to nominal statistical significance of any one model) is evaluated by cross-validation prediction. Predictions are based on standard Bayesian model averaging—weighted model averaging: the models identified are evaluated according to their relative data-based probabilities of model fit, and these probabilities provide weights to use in averaging predictions for the hold-out (or future) tumor samples.

Analysis of sensitivity and specificity in the prediction of platinum response in the training set was performed by using ROC curve to define estimated sensitivity and specificity with respect to each prediction of platinum response. The percent accuracy of the models for the validation set (n=36) was determined by the predicted probability of sensitivity and specificity determined by the ROC curve (probability=0.47) for the training set. The analysis approach for the prediction of oncogenic pathway deregulation has been previously described.³⁶

Cell lines and RNA extraction—The ovarian cancer cell lines, OV90, TOV21G, and TOV112D were grown as recommended by the supplier (ATCC, Rockville, Md.). FUOV1, a human ovarian carcinoma, was grown according to the supplier (DSMZ, Braunschweig, Germany). Eight additional cell lines (C13, OV2008, A2780CP, A2780S, IGROV1, T8, OVCAR5 and IMCC3) were provided by Dr. Patricia Kruk, Department of Pathology, College of Medicine (University of South Florida, Tampa, Fla.). These eight cell lines were grown in RPMI 1640 supplemented with 10% Fetal Bovine Serum, 1% Sodium pyruvate, and 1% non essential amino acids. All tissue culture reagents were obtained from Sigma Aldrich (St. Louis, Mo.). Total RNA was extracted from each cell line and assayed on the Human 133 plus 2.0 arrays.

Cell proliferation assays—Assays measuring cell proliferation and the effects of targeted agents have been described previously³⁶. Briefly, growth curves for the ovarian cancer cell lines were carried out by plating 300-4000 cells per well of a 96-well plate. The growth of cells at 12 hr time points (from t=12 hrs) was determined using the CellTiter 96 Aqueous One Solution Cell Proliferation Assay Kit by Promega, which is a colorimetric method for determining the number of growing cells. Sensitivity to a Src inhibitor (SU6656), CDK/E2F inhibitor (CYC202/R-Roscovitine) and Cisplatin was determined by quantifying the percentage reduction in growth (versus DMSO controls) at 120 hr using a standard MTS (3-(4,5-dimethylthiazol-2-yl)-5-(3-carboxymethoxyphenyl)-2-(4-sulphophenyl)-2H-tetrazolium) colorimetric assay (Promega). Concentrations used for individual and combination treatments were from 0-50 uM for SU6656, CYC202/R-Roscovitine, and Cisplatin. The degree of proliferation inhibition was plotted as a function of probability of Src pathway activation or E2F3 pathway activation. A linear regression analysis demonstrates statistically significant relationships between percent response and probability of Src activity. Significant relationships included p<0.001 between cisplatin plus SU6656 versus Cisplatin alone, p=0.0003 between Cisplatin plus SU6656 versus SU6656 alone and p=0.01 for Cisplain versus SU6656 in relationship to probability of Src activity. A linear regression analysis of inhibition of proliferation plotted as a function of E2F3 pathway activity demonstrates statistically significant (p=0.02) relationship only between roscovatine and probability of E2F3 activity.

Gene Expression Profiles that Predict Platinum Response

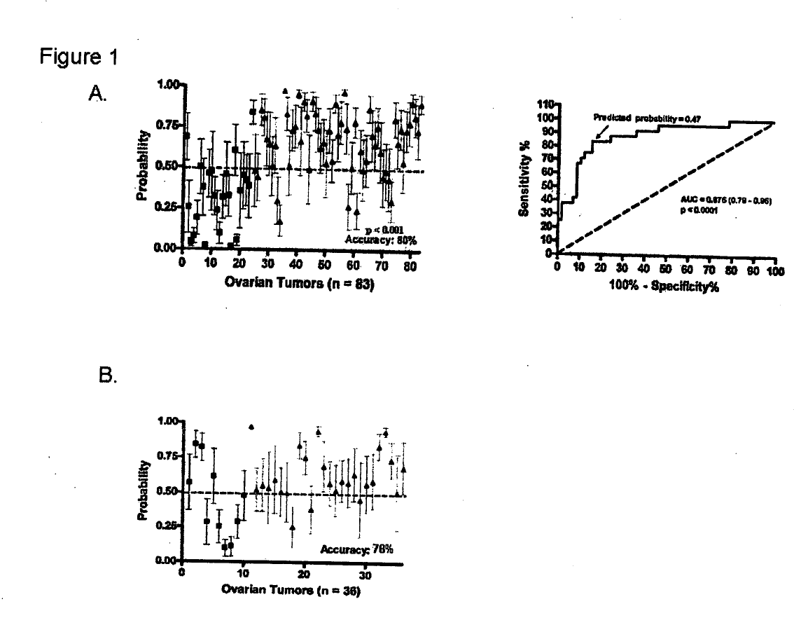

With the ultimate objective of developing a strategy for determining the most appropriate therapy for an individual patient with ovarian cancer, we developed a predictive tool that identifies patients with platinum-resistant disease at the time of initial diagnosis. The 83 sample training set was used to identify a gene expression pattern that could predict clinical outcome. Using a cut-off of 0.47 predicted probability of response, as determined by ROC curve analysis (FIG. 1A, Right panel), platinum response in patients was predicted accurately in 70 out of 83 samples, achieving an overall accuracy of 84.3% (specificity of 85% and sensitivity of 83%) (FIG. 1A). Applying a Mann-Whitney U test for statistical significance (p<0.001) demonstrates the capacity of the predictor to distinguish non responders from responder patients.

A validation of the predictive performance of the gene expression model was performed on a randomly generated set of 36 samples in order to evaluate the ability of the model to predict platinum response. Both training and validation sets were balanced with respect to platinum response rates seen in the clinic (i.e., approximately 70% complete responders). Based on the cut off of 0.47 as defined in the training set (FIG. 1B), it is evident that the predicted platinum response in the training set performs well to predict the response within the separate validation set (78% accuracy). When other clinical variables, such as debulking status or CA-125 were included in the Shotgun Stochastic Search (SSS) to determine platinum response predictions, there was no effect on the predicted accuracy or gene content of the models, suggesting that the signature of platinum response is independent of other clinical variables.

Based on these results, we conclude that it is possible to develop gene expression profiles that have the capacity to predict response to platinum-based chemotherapy and thus serve as a mechanism to stratify patients with respect to treatment. While the ability to identify responsive patients is not likely a primary goal, a capacity to identify the patients resistant to platinum therapy would be a significant benefit in guiding more effective treatment for these patients. In this context, an emphasis on the specificity of predicting resistance might be the most appropriate goal.

A total of 1727 genes were included in the averaged predictive model and the 100 genes most weighted in achieving the prediction are listed in Table 2. Analysis of Gene Ontology categories represented by these genes is depicted in Table 3. The analysis reveals an enrichment for genes reflecting cell proliferation and cell growth, certainly consistent with a mechanism of action of cytotoxic chemotherapeutic agents such as cisplatin and taxol that generally are directed at the proliferative capacity of the cancer cell.

Identifying Therapeutic Options for Patients with De-novo Platinum-resistant Ovarian Cancer

The development of a predictor that can identify patients likely to be resistant to primary platinum therapy provides an opportunity to effectively identify the population most likely to benefit from additional therapeutic intervention. The challenge is determining what other therapies might benefit these patients. While in principle it might be possible to use the gene expression data to deduce the critical biological distinction(s) that predict platinum response, in practice this is difficult due to our limited knowledge of the integration of biological pathways and systems. We believe an alternative strategy is one that makes use of an ability to profile the status of various oncogenic signaling pathways within the tumor. We have recently described the development of gene expression signatures that reflect the activation status of several oncogenic pathways and have shown that these signatures can evaluate the status of the pathways in a series of tumor samples, providing a prediction of relative probability of pathway deregulation of each tumor.³⁶

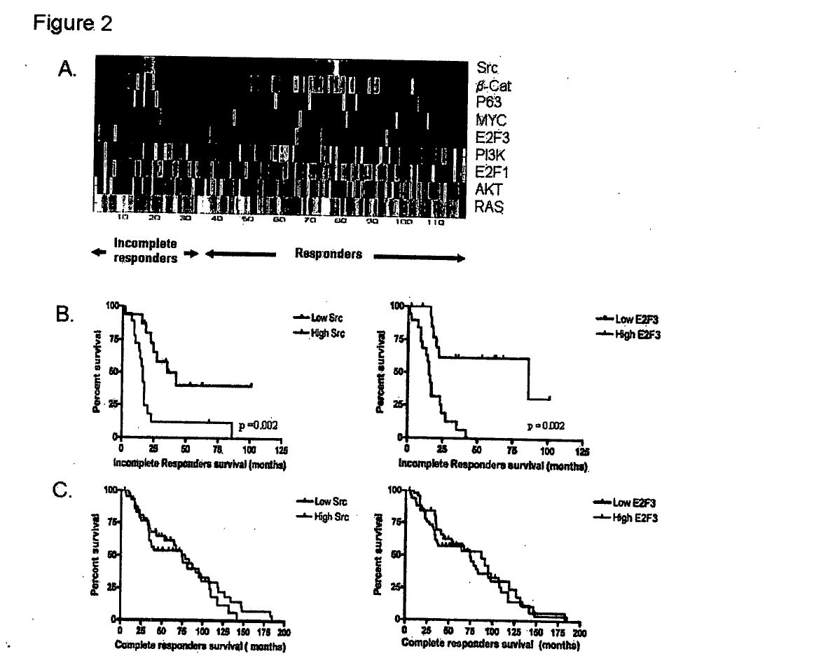

To explore the potential for employing this as an approach to identify new therapeutic options, we made use of the previously developed signatures to predict the status of these pathways in the tumors. In each case, the probability of pathway activation in a given tumor is predicted from the signature developed by expression of the activating oncogene in quiescent epithelial cell cultures. Evidence for high probability of pathway activation is indicated by red and low probability by blue (FIG. 2A). Initial analyses revealed that a substantial number of the tumors exhibit Src pathway deregulation. In FIG. 2A the tumor samples are sorted based on the predicted level of Src activity. The Kaplan-Meier survival analysis in FIG. 2B illustrates further that those patients with deregulated Src pathway also exhibit the worst prognosis. However in complete responders, there was no evident relationship between Src and E2F3 pathway deregulation and survival (FIG. 2C). An examination of other pathways in the context of the Src pathway deregulation revealed Myc and E2F3 to be frequently deregulated in the tumors lacking Src activity. Although Myc pathway deregulation does not link with available therapeutics, E2F3 deregulation does suggest an opportunity for use of a CDK inhibitor. We further explored the potential of these two pathway signatures (Src and E2F3) to direct the use of inhibitors that target these pathways.

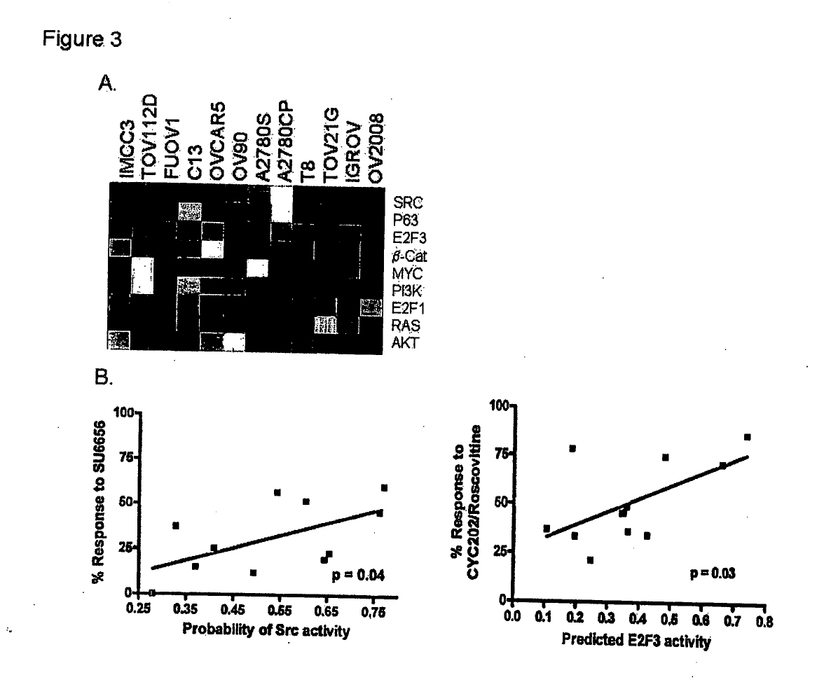

In parallel with the determination of pathway status in the tumors, we characterized the status of the pathways in a series of ovarian cancer cell lines (FIG. 3A). This analysis provides a baseline measure of the status of these pathways that can be compared to the sensitivity of the cells to therapeutic drugs known to target specific activities within given oncogenic pathways. The goal is to determine if a cell line is sensitive to a drug based on the knowledge of the pathway deregulation within that cell. For the Src pathway we made use of a Src-specific inhibitor (SU6656) and for the E2F3 pathway we made use of a CDK inhibitor (CYC202/R-Roscovitine). The ability of these agents to inhibit growth of the ovarian cancer cell lines was assessed using assays of cell proliferation. In FIG. 3B, a clear and statistically significant relationship can be seen between prediction of either Src or E2F3 pathway deregulation and sensitivity to the respective therapeutic of that pathway. As such, it is evident from these results that predicted pathway deregulation predicts sensitivity to the pathway-specific therapeutic agent.

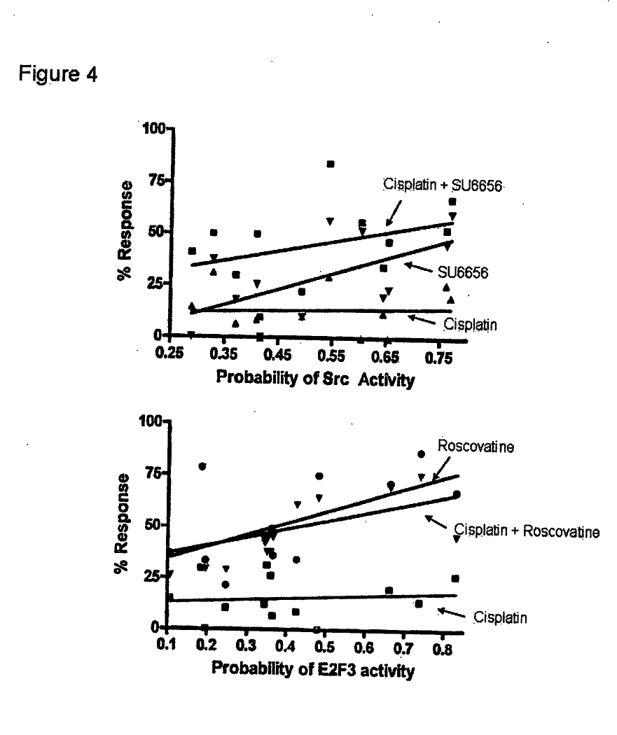

Although the goal of the use of pathway predictions is to identify options for patients with platinum-resistant ovarian cancer, it is nevertheless true that most of the patients with platinum-resistant disease will show some evidence of response to platinum therapy. The utilization of targeted therapeutics such as the Src or CDK inhibitor likely would be in conjunction with standard cytotoxic chemotherapies such as carboplatin and paclitaxel. We have further investigated the extent to which there may be an additive effect of combined therapies. A collection of ovarian cancer cell lines were assayed for sensitivity to cisplatin either with or without SU6656 or CYC202/R-Roscovitine. In FIG. 4, the response was plotted as a function of pathway prediction (either Src or E2F3),and as seen previously, there is a relationship between pathway deregulation and SU6656 or CYC202/R-Roscovitine drug sensitivity. In contrast, there was no evident relationship between pathway deregulation and cisplatin sensitivity. Nevertheless, there was evidence for a greater sensitivity to the combination of cisplatin and SU6656 compared to either agent alone, whereas there was no evident added benefit of cisplatin combined with roscovitine, versus roscovitine alone.

Taken together, these results demonstrate a capacity of a pathway signature to not only predict deregulation of the pathway but to.also predict sensitivity to therapeutic agents that target the corresponding pathways. We suggest this is a viable approach for directing the use of various therapeutic agents.

Discussion

Treatment of patients with advanced stage ovarian cancer is empiric and almost all patients receive a platinum drug, usually with a taxane. Although many patients have a complete clinical response to platinum-based primary therapy, a significant fraction of patients either have an incomplete response or develop progression of disease during primary therapy. Recently several groups have utilized genomic approaches to delineate genes that may impact ovarian cancer platinum-responsiveness.²⁴⁻²⁷ Although we can identify some commonality of gene family/function (i.e., zinc finger proteins, ubiquitin specific proteases, protein phosphatases, and DNA mismatch repair genes) between our platinum predictor and those of others,²⁴⁻²⁷ common genes do not appear to be represented which could be limited due to the use of cDNA-based microarrays by other groups.

Strategies for the treatment of patients determined to be resistant to platinum-based chemotherapy involve the use of various empiric-based salvage chemotherapy agents that often have only marginal benefit. Although it is possible that, based on knowledge that the patient is unlikely to benefit from platinum therapy, initiation of salvage agents as first-line therapy would achieve a greater benefit, we believe a more effective strategy may be the use of agents that target components of pathways that are seen to be deregulated in individual cancers. Thus, the therapeutic strategy is tailored to the individual patient based on knowledge of the unique molecular alterations in their tumor.

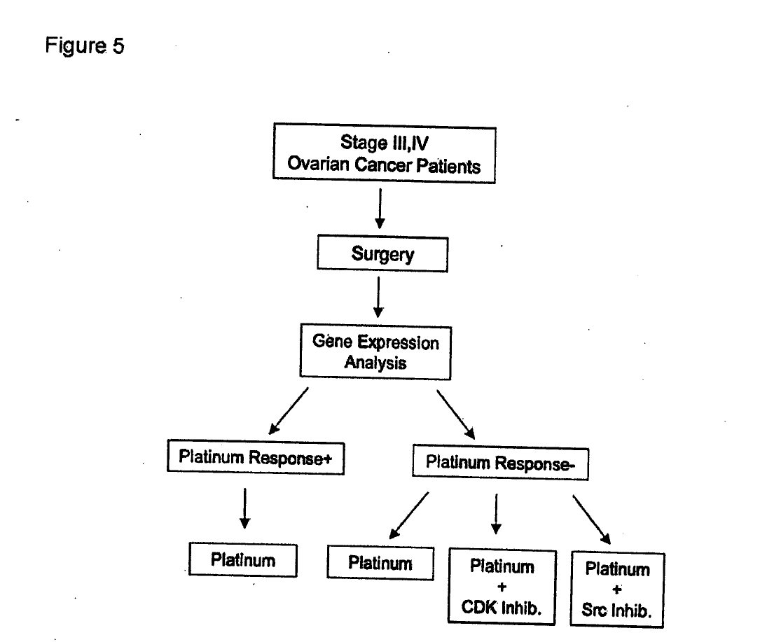

Individualizing treatments by identifying those patients unlikely to respond fully to the primary platinum-based therapy coupled with an ability to identify characteristics unique to this group of patients can direct the use of novel therapeutic strategies. This truly represents a move towards the goal of personalized treatment. An outline of the approach afforded by these developments is summarized in FIG. 5. The capacity to predict likely response to platinum chemotherapy based on gene expression data obtained from the primary tumor can identify those patients most appropriate for additional therapies. The purpose of this assessment is not to direct the use of primary platinum-based chemotherapy but rather to identify that subset of patients who most likely will benefit from additional therapies. The use of pathway predictions provides a basis for utilization of drugs specific to the deregulated pathway in patients predicted to have platinum-resistant disease. In FIG. 5, this might involve a choice of either a Src inhibitor or a cyclin kinase inhibitor based on the observation that these two pathways dominate ovarian cancers and the results that demonstrate a capacity of these pathway predictors to also predict sensitivity to these agents. Given the fact that most patients demonstrate some (if not complete) response to platinum, we would expect that for now, all patients would still receive standard platinum therapy, but patients predicted to have an incomplete response to platinum would also receive a targeted therapeutic.

We believe the approach described here, using gene expression profiles that predict primary chemotherapy response coupled with expression data that identifies oncogenic pathway deregulation to stratify patients to the most appropriate treatment regimen, represents an important step towards the goal of personalized cancer treatment. We further suggest that a major benefit of this approach (and in particular the use of pathway information to guide the use of targeted therapeutics), is the capacity to ultimately direct the formulation of combinations of therapies—multiple drugs that target multiple pathways—based on information that details the state of activity of the pathways.

### Example 2

### Development and Characterization of Gene Expression Profiles that Determine Response to Topotecan Chemotherapy for Ovarian Cancer

Material and Methods

MIAME (minimal information about a microarray experiment)-compliant information regarding the analyses performed here, as defined in the guidelines established by MGED (www.mged.org), is detailed in the following sections.

Tissues—We measured expression of 22,283 genes in 12 ovarian cancer cell lines and 48 advanced (FIGO stage III/IV) serous epithelial ovarian carcinomas using Affymetrix U1 33A GeneChips. All ovarian cancers were obtained at initial cytoreductive surgery from patients treated at H. Lee Moffitt Cancer Center & Research Institute or Duke University Medical Center. All patients received primary platinum-based adjuvant chemotherapy and went on to demonstrate persistent or recurrent disease. All tissues were collected under the auspices of a respective institutional IRB approved protocol with written informed consent.

Classification of topotecan response—Response to therapy was retrospectively evaluated from the medical record using standard criteria for patients with measurable disease, based upon WHO guidelines (Miller A B, et al., Cancer 1981;47:207-14). CA-125 was used to classify responses only in the absence of a measurable lesion; CA-125 response criteria were based on established guidelines (Miller A B, et al. Cancer 1981;47:207-14; Rustin G J, et al., Ann. Onco. 110:21-27, 1999). A complete response was defined as a complete disappearance of all measurable and assessable disease or, in the absence of measurable lesions, a normalization of the CA-125 level following topotecan therapy. A complete response (CR) was defined as a complete disappearance of all measurable and assessable disease or, in the absence of measurable lesions, a normalization of the CA-125 level following topotecan therapy. A partial response (PR) was considered a 50% or greater reduction in the product obtained from measurement of each bi-dimensional lesion for at least 4 weeks or a drop in the CA-125 by at least 50% for at least 4 weeks. Progressive disease (PD) was defined as a 50% or greater increase in the product from any lesion documented within 8 weeks of initiation of therapy, the appearance of any new lesion within 8 weeks of initiation of therapy, or any increase in the CA-125 from baseline at initiation of therapy. Stable disease (SD) was defined as disease not meeting any of the above criteria.

For the purposes of the array analysis, a topotecan responder included patients that demonstrated CR, PR, or SD. Topotecan non-responders were considered patients that demonstrated PD on topotecan therapy.

Microarray analysis—Frozen tissue samples were embedded in OCT medium and sections were cut and mounted on slides. The slides were stained with hematoxylin and eosin to assure that samples included greater than 70% cancer. Approximately 30 mg of tissue was added to a chilled BioPulverizer H tube (Bio101). Lysis buffer from the Qiagen Rneasy Mini kit was added and the tissue homogenized for 20 seconds in a Mini-Beadbeater (Biospec Products). Tubes were spun briefly to pellet the garnet mixture and reduce foam. The lysate was transferred to a new 1.5 ml tube using a syringe and 21 gauge needle, followed by passage through the needle 10 times to shear genomic DNA. Total RNA was extracted using the Qiagen Rneasy Mini kit. Two extractions were performed for each cancer and the total RNA pooled at the end of the Rneasy protocol, followed by a precipitation step to reduce volume.

Cell and RNA preparation—Full details of development of gene expression signatures representing deregulation of oncogenic pathways are described in our recent publication.³⁶ Total RNA was extracted for cell lines using the Qlashredder and Qiagen Rneasy Mini kits. Quality of the RNA was checked by an Agilent 2100 Bioanalyzer. The targets for Affymetrix DNA microarray analysis were prepared according to the manufacturer's instructions. Biotin-labeled cRNA, produced by in vitro transcription, was fragmented and hybridized to the Affymetrix U133A Gene Chip arrays (www.affymetrix.com_products_arrays specific Hu133A.affx) at 45° C. for 16 hr and then washed and stained using the GeneChip Fluidics. The arrays were scanned by a GeneArray Scanner and patterns of hybridization detected as light emitted from the fluorescent reporter groups incorporated into the target and hybridized to oligonucleotide probes.

Cell Culture—All liquid media as well as the Thiazolyl Blue Tetrazolium Bromide were purchased from Sigma Aldrich (St. Louis, Mo.). The Src inhibitor SU6656 and the Topotecan hydrochloride were purchased from Calbiochem (San Diego, Calif.). The ovarian cancer cell lines, OV90, OVCA5, TOV21G, and TOV12D were grown as recommended by the supplier (ATCC, Rockville, Md.). FUOV1, a human ovarian carcinoma, was grown according to the supplier (DSMZ; Braunschweig, Germany). Seven additional cell lines (C13, OV2008, A2780CP, A2780S, IGROV1, T8, IMCC3) were provided by Dr. Patricia Kruk, College of Medicine (University of South Florida, Fla.). All of those seven cell lines were grown in RPMI 1640, supplemented with 10% Fetal Bovine Serum, 1% sodium pyruvate, and 1% non essential amino acids. All tissue culture reagents were obtained from Sigma (UK).

Cell proliferation assays—Growth curves for cells were produces out by plating at 500-10,000 cells per well of a 96-well plate. The growth of cells at 12 hr time points (from t=12 hrs) was determined using the CellTiter 96 Aqueous One 23 Solution Cell Proliferation Assay Kit by Promega, which is a calorimetric method for determining the number of growing cells. The growth curves plot the growth rate of cells on the Y-axis and time on the X-axis for each concentration of drug tested against each cell fine. Cumulatively, these experiments determined the concentration of cells to use for each cell line, as well as the dosing range of the inhibitors. The dose-response curves in our experiments plot the percent of cell population responding to the chemotherapy on the Y-axis and concentration of drug on the X-axis for each cell line. Sensitivity to topotecan and a Src inhibitor (SU6656), both single alone and combined was determined by quantifying the percent reduction in growth (versus DMSO controls) at 96 hrs. Concentrations used were 300 nM-10 μM (S U6656) and 100 nM-10 uM (topotecan). All experiments were repeated in triplicate.

Statistical analysis—For microarray analysis experiments, expression was calculated using the robust multi-array average (RMA) algorithm³¹ implemented in the Bioconductor (http://www.bioconductor.org) extensions to the R statistical programming environment (Ihaka R, et al., J. Comput. Graph. Stat. 1996; 5:299-314). RMA generates log-2 scaled measures of expression using a linear model robustly fit to background-corrected and quantile-normalized probe-level expression data and has been shown to have a better ability to detect differential expression in spike-in experiments (Bolstad B M, et al., Bioinformatics 2003; 19:185-193). The 22,283 probe sets were screened to remove 68 control genes, those with a small variance and those expressed at low levels. The core methodology for predicting response to topotecan uses statistical classification and prediction tree models, and the gene expression data (RMA values) enter into these models in the form of metagenes. As described in published articles, for example, Huang E, et al., Lancet 2003; 361:1590-1596; Pittman J, et al., Proc. Nat'l. Acad. Sci. 2004; 101:8431-36; and Pittman J, et al., Biostatistics 2004 Octuber;5(4):587-601, metagenes represent the aggregate patterns of variation of subsets of potentially related genes. In this example, metagenes are constructed as the first principal components (singular factors) of clusters of genes created by using k-means clustering. Predictions are based on weighted averages across multiple candidate tree models containing metagenes that are used to predict topotecan response. Iterative out-of-sample, cross-validation predictions (leaving each tumor out of the data set one at a time, refitting the model by selecting both the metagene factors and the partitions used from the remaining tumors, and then predicting the hold-out case) are used to test the predictive value of the model. Full details of the statistical approach, including creation of metagenes, are described in published articles, for example, Huang E, et al., Lancet 2003; 361:1590-1596; Pittman J, et al., Proc. Nat'l. Acad. Sci. 2004; 101:8431-36; and Pittman J, et al., Biostatistics 2004 October;5(4):587-601.

In the analysis of the various oncogenic pathways, analysis of expression data was done as previously described in Bild A, et al., Nature 439:353-357, 2006 and West M, et al., Proc. Natl. Acad. Sci. USA 2001;98(20):11462-7). In brief, a library of gene expression signatures was created by infection of primary human normal epithelial cells with adenovirus expressing either human c-Myc, activated H-Ras, human c-Src, human E2F3, or activated β-catenin. Gene expression data was filtered prior to statistical modeling that excluded probesets with signals present at background noise levels, and for probesets that do not vary significantly across samples. Each oncogenic signature summarizes its constituent genes as a single expression profile, and is derived as the first principal component of that set of genes (the factor corresponding to the largest singular value) as determined by a singular value decomposition. Given a training set of expression vectors (metagenes) representing two biological states (i.e., GFP and Src), a binary probit regression model is estimated using Bayesian methods. The ovarian tumor samples were applied as a separate validation data set, which allows one to evaluate the predictive probabilities of each of the two states for each oncogenic pathway in the validation set. Hierarchical clustering of tumor predictions was performed using Gene Cluster 3.0 (Eisen, M. B.,et al., Proc. Natl. Acad. Sci. USA 1998; 95(25):14863-8). Genes and tumors were clustered using average linkage with the centered correlation similarity metric. For cell lines analysis of response to therapy with topotecan and src inhibitor, the percent response was calculated as follow: Percent response=1−Absorbency of control group (Absorbency of experimental group×100%. Statistical analysis for significance of the difference included a paired two-tailed t-test.

Results

The major motivation for this study is the characterization of the genomic basis of epithelial ovarian cancer response to topotecan chemotherapy. We hope to develop a preliminary predictive tool that may identify patients most likely to benefit from topotecan therapy for recurrent or persistent ovarian cancer at the time of initial diagnosis. Further, by defining the oncogenic pathways that contribute to topotecan resistance we hope to identify additional therapeutic options for patients predicted to have ovarian cancer resistant to single-agent topotecan therapy.

We measured expression of 22,283 genes in 48 advanced (FIGO stage III/IV) serous epithelial ovarian carcinomas using Affymetrix U133A GeneChips. All ovarian cancers were obtained at initial cytoreductive surgery from patients treated at H. Lee Moffitt Cancer Center & Research Institute or Duke University Medical Center. Response to therapy was evaluated from the medical record and patients were classified as either topotecan responders or non responders, by criteria described above. From the group of 48 patients analyzed, 30 were classified as topotecan responders and 18 as non-responders.

Gene Expression Profiles that Predict Topotecan Response

Our recent work in breast cancer has described the development of predictive models that make use of multiple forms of genomic and clinical data to achieve more accurate predictions of individual risk of recurrence of disease (Huang E, et al., Lancet 2003; 361:1590-1596; Pittman J, et al., Proc. Nat'l. Acad. Sci. 2004; 101:8431-36; and Pittman J, et al., Biostatistics 2004 October;5(4):587-601). The method for selecting multiple gene expression patterns, that we term metagenes, makes use of Bayesian-based classification and regression tree analysis. Metagenes are derived from a clustering of the original gene expression data in which genes with similar expression patterns are grouped together. The expression data from the genes in each cluster are then summarized as the first principal component of the expression data, i.e., the metagene for the cluster. The metagenes are sampled by the classification trees to generate partitions of the samples into more and more homogeneous subgroups that in this case reflect the response to topotecan therapy. At each node of a tree, the subset of patients is divided in two based on a threshold value of a chosen metagene, and the heterogeneity within the groups is reduced.

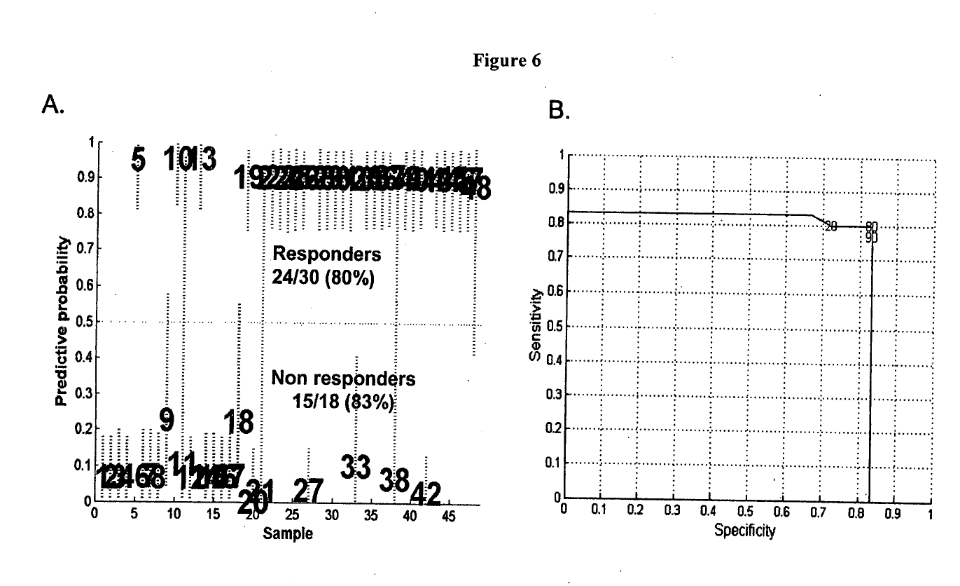

Bayesian classification tree models were developed that included metagenes, and a leave-one-out cross validation produced a predictive profile of 261 genes with an overall accuracy of 81% for correctly predicting response to topotecan (24130 (80%) for predicting responders, and 15118 (83%) for predicting non-responders). Genes included in the predictive profile are listed in Table 5. The predictive summary for the samples of ovarian cancers is demonstrated in FIG. 6A. The predicted probability of response is plotted for each patient along with the statistical uncertainty in the prediction. The latter derives from the uncertainties evident across the array of candidate trees generated in the analysis. An examination of the estimated receiver operator characteristic (ROC) curves for response indicates a capacity to achieve up to 80% sensitivity with 83% specificity in predicting topotecan responders (FIG. 6B).

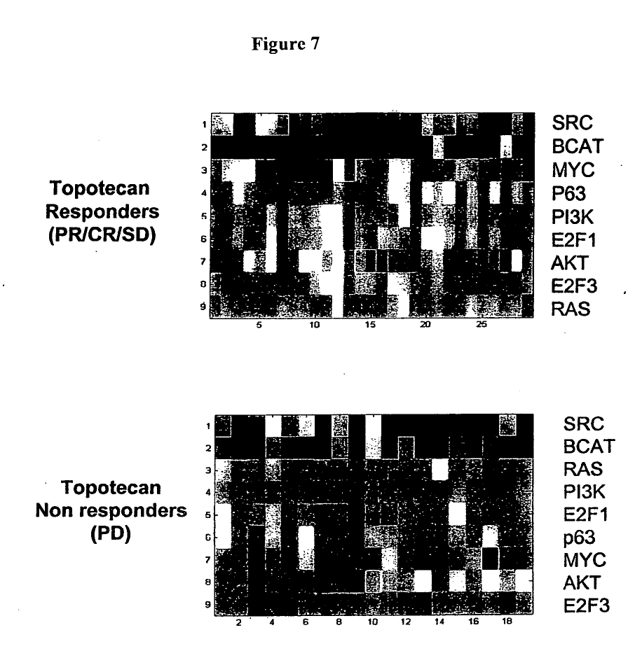

Identifying therapeutic options for topotecan resistant patients—Although a gene expression profile that predicts topotecan response may facilitate the identification of patients likely not to benefit from single-agent topotecan therapy, it does little to aid selection of alternate therapeutic approaches. In an effort to identify therapeutic options for topotecan-resistant patients we have taken advantage of our recent work, which describes the development of gene expression signatures that reflect the activation status of several oncogenic pathways. We have applied these signatures to evaluate the status of pathways in the 48 primary ovarian cancer samples resected from patients who later went on to experience recurrent or persistent disease treated with topotecan. This approach provides a prediction of the relative probability of pathway deregulation of each of the 48 primary ovarian cancers based on previously developed signatures. This analysis revealed that the src and beta-catenin pathways were activated in 55% (10/18) and 77% (14/18) respectively, of primary cancers from patients who went onto demonstrate topotecan-resistant recurrent or persistent disease (FIG. 7).

In parallel with the determination of pathway status in primary specimens, 12 ovarian cancer cell lines were subject to assays with topotecan as well as a drug known to target a specific activity within the src oncogenic pathway, SU6656. If src deregulation contributes to the topotecan-resistant phenotype, then inhibition of the pathway may effect a reversal of topotecan resistance. The goal was to directly demonstrate that a cell line is sensitive to a drug based on the knowledge of the pathway deregulation within that cell. For the src pathway we made use of a Src-specific inhibitor (SU6656). In each case, we employed growth inhibition as the assay. The Src-specific inhibitor, SU6656 increases ovarian cancer cell line sensitivity to topotecan, and as shown in FIG. 8 a clear relationship was demonstrated between predicted src-pathway deregulation and response of those ovarian cancer cells to both src-inhibitor alone (p=0.03) and to combined src-inhibitor plus topotecan (p=0.05). Of interest, the benefit of adding SU6656 to topotecan (in terms of cell responsiveness) increased with predicted src-pathway activity (p=0.01). Importantly, a comparison of the drug inhibition results with predictions of other pathways failed to demonstrate a significant correlation.

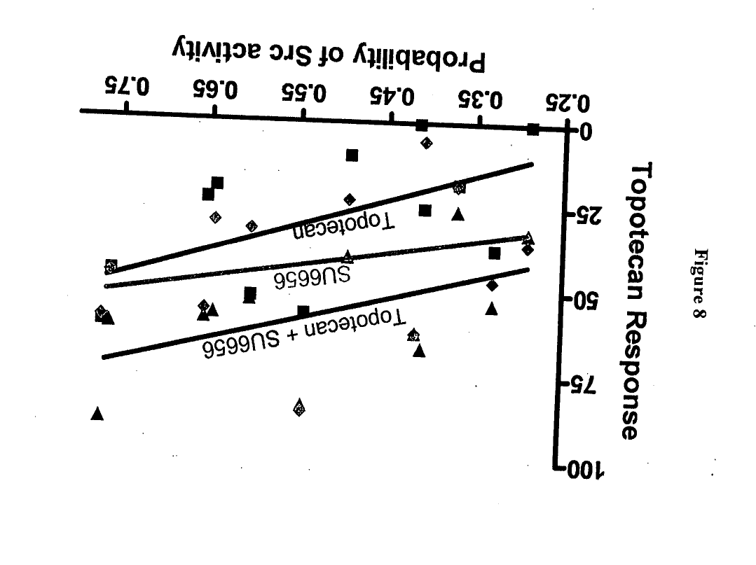

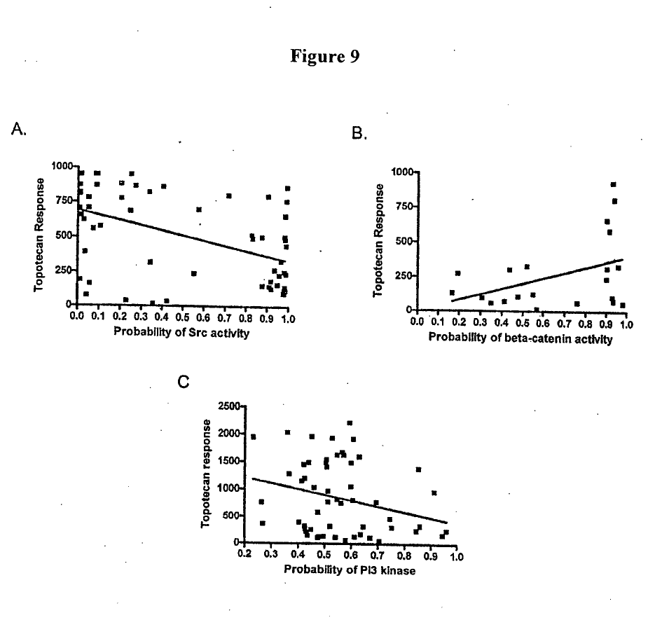

In an effort to further explore the utility of oncogenic pathway deregulation as a predictor of response to topotecan-based therapy for other human cancers we evaluated published genomic and chemotherapeutic response data for the 60 human cancer cell lines (NCI-60) used in “NCI In Vitro Cell Line Screening Project” (http://www.dtp.nei.nih.gov/webdata.html). Consistent with our findings in ovarian cancer cell lines, predicted deregulation of the src pathway was highly correlated with topotecan response (p=0.0002) of the set of 60 human cancer cell lines that represent the NCI In Vitro Cell Line Screening Project (FIG. 9A). Additionally, in the NCI-60 cells a correlation was identified between predicted deregulation of the PI3 Kinase pathways and topotecan response (p=0.04, FIG. 9B). Of interest, predicted activation of the β-catenin pathway was also associated with topotecan response in the ovarian, renal, prostate and colon cell lines within the NCI-60 (p=0.04), though not with breast, lung, leukemia, CNS and melanoma cell lines (FIG. 9C).

### Example 3

### Gene Expression Profiles that Direct Salvage Therapy for Ovarian Cancer

Material and Methods

Topotecan-response predictor—To develop a gene expression based predictor of sensitivity/resistance from the pharmacologic data used in the NCI-60 drug screen studies, we chose cell lines within the NCI-60 panel that would represent the extremes of sensitivity to topotecan. The (21 og10) G150, TGI and LC50 data was used to populate a matrix with MATLAB software, with the relevant expression data for the individual cell lines. Where multiple entries for topotecan existed (by NCS number), the entry with the largest number of replicates was included. Incomplete data were assigned asNaN (not a number) for statistical purposes. Since the TGI and LC50 dose represent the cytostatic and cytotoxic levels of any given drug, cell lines with low LC50 and TGI were considered sensitive and those with the highest TGI and LC50 were considered resistant. The log transformed TGI and LC50 doses of the sensitive and resistant subsets was then correlated with the respective GI50 data to ascertain consistency between the TGI, LC50 and GI50 data. Because the G150 data is non-gaussian with many values around 4, a variance fixed t-test was used to calculate significance. Relevant expression data (updated data available on the Affymetrix U95A2 GeneChip) for the solid tumor cell lines and the respective pharmacological data for topotecan was downloaded from the website (http://dtp.nci.nih.gov/docs/cancer/cancer data.html). The topotecan sensitivity and resistance data from the selected solid tumor NCI-60 cell lines was then used in a supervised analysis using binary regression analysis to develop a model of topotecan response.

Tissues—We measured expression of 22,283 genes in 12 ovarian cancer cell lines and 48 advanced (FIGO stage III/IV) serous epithelial ovarian carcinomas using Affymetrix U133A GeneChips. All ovarian cancers were obtained at initial cytoreductive surgery from patients treated at H. Lee Moffitt Cancer Center & Research Institute or Duke University Medical Center. All patients received topotecan as salvage chemotherapy after initial platinum based therapy. All tissues were collected under the auspices of a respective institutional IRB approved protocol with written informed consent.

Classification of topotecan response in tumors—Response to therapy was retrospectively evaluated from the medical record using standard criteria for patients with measurable disease, based upon WHO guidelines ((Miller A B, et al., Cancer 1981;47:207-14). CA-125 was used to classify responses only in the absence of a measurable lesion; CA-125 response criteria were based on established guidelines (Miller A B, et al. Cancer 1981;47:207-14; Rustin G J, et al., Ann. Onco. 110:21-27, 1999). A complete responder was defined as a complete disappearance of all measurable and assessable disease or, in the absence of measurable lesions, a normalization of the CA-125 level following topotecan therapy. Non-responders/patients with progressive disease (PD) were defined as a 50% o or greater increase in the primary lesion(s) documented within 8 weeks of initiation of therapy or the appearance of any new lesion within 8 weeks of initiation of therapy.

Microarray analysis—Frozen tissue samples were embedded in OCT medium and sections were cut and mounted on slides. The slides were stained with hematoxylin and eosin to assure that samples included greater than 70% cancer. Approximately 30 mg of tissue was added to a chilled BioPulverizer H tube (Bio101). Lysis buffer from the Qiagen Rneasy Mini kit was added and the tissue homogenized for 20 seconds in a Mini-Beadbeater (Biospec Products). Tubes were spun briefly to pellet the garnet mixture and reduce foam. The lysate was transferred to a new 1.5 ml tube using a syringe and 21 gauge needle, followed by passage through the needle 10 times to shear genomic DNA. Total RNA was extracted using the Qiagen RNeasy Mini kit. Two extractions were performed for each cancer and the total RNA pooled at the end of the Rneasy protocol, followed by a precipitation step to reduce volume. MIAME (minimal information about a microarray experiment)-compliant information regarding the analyses performed here, as defined in the guidelines established by MGED (www.mged.org), is detailed in the following sections.

Cell and RNA preparation—Full details of development of gene expression signatures representing deregulation of oncogenic pathways are described in.³⁶ Total RNA was extracted for cell lines using the Qiashredder and Qiagen Rneasy Mini kits. Quality of the RNA was checked by an Agilent 2100 Bioanalyzer. The targets for Affymetrix DNA microarray analysis were prepared according to the manufacturer's instructions. Biotin-labeled cRNA, produced by in vitro transcription, was fragmented and hybridized to the Affymetrix U133A GeneChip arrays (www.affymetrix.com_products_arrays specific_Hu133A.affx) at 45° C. for 16 hours and then washed and stained using the GeneChip Fluidics. The arrays were scanned by a GeneArray Scanner and patterns of hybridization detected as light emitted from the fluorescent reporter groups incorporated into the target and hybridized to oligonucleotide probes.

Cell culture—All liquid media as well as the Thiazolyl Blue Tetrazolium Bromide were purchased from Sigma Aldrich (St. Louis, Mo.). The Src inhibitor SU6656 and the Topotecan hydrochloride were purchased from Calbiochem (San Diego, Calif.). The ovarian cancer cell lines, OV90, OVCA5, TOV21G, and TOV 112D were grown as recommended by the supplier (ATCC, Rockville, Md.). FUOV 1, a human ovarian carcinoma, was grown according to the supplier (DSMZ, Braunschweig, Germany). Seven additional cell lines (C13, OV2008, A2780CP, A2780S, IGROV 1, T8, IMCC3) were provided by Dr. Patricia Kruk, College of Medicine (University of South Florida, Fla.). All of those seven cell lines were grown in RPMI 1640, supplemented with 10% Fetal Bovine Serum, 1% sodium pyruvate, and 1% non essential amino acids. All tissue culture reagents were obtained from Sigma (UK).

Cell proliferation assays—Growth curves for cells were produced by plating 500-10,000 cells per well in 96-well plates. The growth of cells at 12 hour time points (from t=12 hrs) was determined using the CellTiter 96 Aqueous One 23 Solution Cell Proliferation Assay Kit by Promega, which is a colorimetric method for determining the number of growing cells. The growth curves plot the growth rate of cells on the Y-axis and time on the X-axis for each concentration of drug tested against each cell line. Cumulatively, these experiments determined the concentration of cells to use for each cell line, as well as the dosing range of the inhibitors. The dose-response curves in our experiments plot the percent of cell population responding to the chemotherapy on the Y-axis and concentration of drug on the X-axis for each cell line. Sensitivity to topotecan, Src inhibitor (SU6656)(both single alone and combined), and R-Roscovitine, a cell cycle inhibitor, was determined by quantifying the percent reduction in growth (versus DMSO controls) at 96 hrs. Concentrations used were 300 nM-10 μM (SU6656), 20-80 μM (R-Roscovitine) and 100 nM -10 μM (topotecan). All experiments were repeated in triplicate.

Statistical analysis—For microarray analysis experiments, expression was calculated using the robust multi-array average (RMA) algorithm³¹ implemented in the Bioconductor (http://www.bioconductor.org) extensions to the R statistical programming environment (Ihaka R, et al., J. Comput. Graph. Stat. 1996; 5:299-314). RMA generates log-2 scaled measures of expression using a linear model robustly fit to background-corrected and quantile-normalized probe-level expression data and has been shown to have a better ability to detect differential expression in spike-in experiments (Bolstad B M, et al.,. Bioinformatics 2003; 19:185-193). The 22,283 probe sets were screened to remove 68 control genes, those with a small variance and those expressed at low levels. The core methodology for predicting response to topotecan uses statistical classification and prediction tree models, and the gene expression data (RMA values) enter into these models in the form of metagenes. As described in published articles, for example, Huang E, et al., Lancet 2003; 361:1590-1596; Pittman J, et al., Proc. Nat'l. Acad. Sci. 2004; 101:8431-36; and Pittman J, et al., Biostatistics 2004 October;5(4):587-601, metagenes represent the aggregate patterns of variation of subsets of potentially related genes. In this example, metagenes are constructed as the first principal components (singular factors) of clusters of genes created by using k-means clustering. Predictions are based on weighted averages across multiple candidate tree models containing metagenes that are used to predict topotecan response. Iterative out-of-sample, cross-validation predictions (leaving each tumor out of the data set one at a time, refitting the model by selecting both the metagene factors and the partitions used from the remaining tumors, and then predicting the hold-out case) are used to test the predictive value of the model. Full details of the statistical approach, including creation of metagenes, are described in published articles, for example, Huang E, et al., Lancet 2003; 361:1590-1596; Pittman J, et al., Proc. Nat'l. Acad. Sci. 2004; 101:8431-36; and Pittman J, et al., Biostatistics 2004 October;5(4):587-601.

In the analysis of the various oncogenic pathways, analysis of expression data was done as previously described in Bild A, et al., Nature 439:353-357, 2006 and West M, et al., Proc. Natl. Acad. Sci. USA 2001;98(20):11462-7. In brief, a library of gene expression signatures was created by infection of primary human normal epithelial cells with adenovirus expressing either human c-Myc, activated H-Ras, human c-Src, human E2F3, or activated β-catenin. Gene expression data was filtered prior to statistical modeling that excluded probesets with signals present at background noise levels, and for probesets that do not vary significantly across samples. Each oncogenic signature summarizes its constituent genes as a single expression profile, and is derived as the first principal component of that set of genes (the factor corresponding to the largest singular value) as determined by a singular value decomposition. Given a training set of expression vectors (metagenes) representing two biological states (i.e., GFP and Src), a binary probit regression model is estimated using Bayesian methods. The ovarian tumor samples were applied as a separate validation data set, which allows one to evaluate the predictive probabilities of each of the two states for each oncogenic pathway in the validation set. Hierarchical clustering of tumor predictions was performed using Gene Cluster 3.0 (Eisen, M. B.,et al., Proc. Natl. Acad. Sci. USA 1998; 95(25):14863-8). Genes and tumors were clustered using average linkage with the centered correlation similarity metric. For cell lines analysis of response to therapy with topotecan and src inhibitor, the percent response was calculated as follow: Percent response=1−Absorbency of control group (Absorbency of experimental group×100%. Statistical analysis for significance of the difference included a paired two-tailed t-test.

Results

The standard protocol for treatment of advanced stage ovarian cancer patients involves a primary regimen of platinum/taxol. Patients that develop resistance are then treated with a variety of second line salvage agents including topotecan, taxol, adriamycin, gemcitabine, cytoxan, and etoposide. Previous work has not provided evidence for clear superiority of one of these salvage agents. As an example, the results of a phase III randomized trial that compared the efficacy of topotecan with paclitaxel showed that the two drugs have similar activity when given as second line therapy. See, for example, publications by W. W. ten Bokkel Huinink.

With the goal of developing a strategy that could effectively identify the most optimal therapeutic options for patients with platinum-resistant epithelial ovarian cancer, we have made use of clinical studies measuring the response to various salvage cytotoxic chemotherapeutic agents, together with microarray generated gene expression data, to develop expression profiles that could predict the potential response to the drugs. This has then been matched with a capacity to identify deregulation of various oncogenic signaling pathways to create a strategy for combining standard chemotherapy drugs with targeted therapeutics in a way that best matches the characteristics of the individual patient.

Development of Gene Expression Profiles that Predict Topotecan Response

We began with studies to predict response to topotecan. We measured expression of 22,283 genes in 48 advanced (FIGO stage III/IV) serous epithelial ovarian carcinomas using Affymetrix U133A GeneChips. All ovarian cancers were obtained at initial cytoreductive surgery from patients treated at H. Lee Moffitt Cancer Center & Research Institute or Duke University Medical Center. Response to therapy was evaluated from the medical record and patients were classified as either topotecan responders or non responders, by criteria described above. From the group of 48 patients analyzed, 30 were classified as topotecan responders and 18 as non-responders.

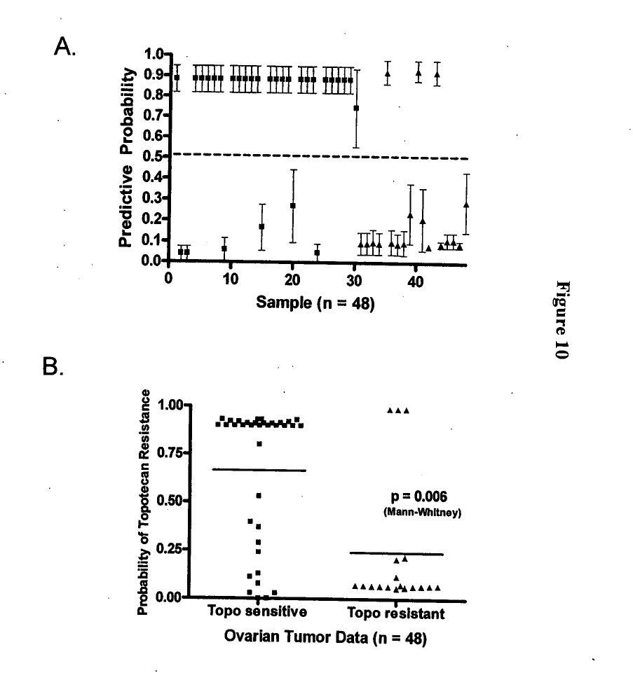

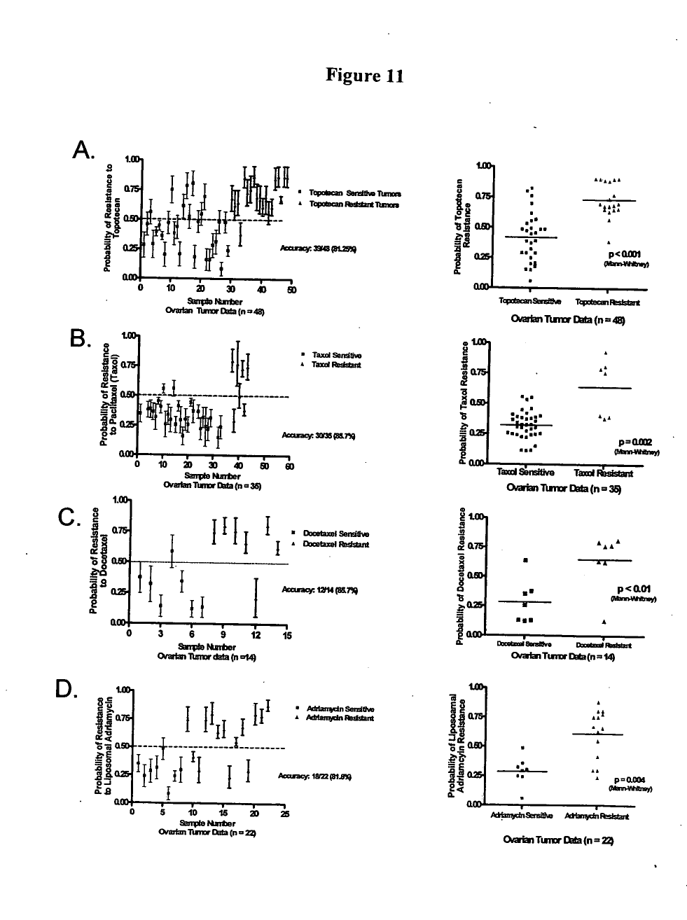

Our recent work in breast cancer has described the development of predictive models that make use of multiple forms of genomic and clinical data to achieve more accurate predictions of individual risk of recurrence of disease (Huang E, et al., Lancet 2003; 361:1590-1596; Pittman J, et al., Proc. Nat'l. Acad. Sci. 2004; 101:8431-36; and Pittman J, et al., Biostatistics 2004 October;5(4):587-601). The method for selecting multiple gene expression patterns, that we term metagenes, makes use of Bayesian-based classification and regression tree analysis. Metagenes are derived from a clustering of the original gene expression data in which genes with similar expression patterns are grouped together. The expression data from the genes in each cluster are then summarized as the first principal component of the expression data, i.e., the metagene for the cluster. The metagenes are sampled by the classification trees to generate partitions of the samples into more and more homogeneous subgroups that in this case reflect the response to topotecan therapy. Bayesian classification tree models were developed that utilized a collection of metagenes that included a total of 261 genes (FIG. 10A). The predictive accuracy of the model, as assessed with a leave-one-out cross validation, was 81% for correctly predicting response to topotecan (FIG. 11B). Further analysis demonstrated a clear statistically significant distinction in predicting responders and non-responders (FIG. 11C).

Utilization of Signatures for Chemotherapy Response Developed from Cancer Cell Lines

Because the majority of advanced stage ovarian cancer patients receive topotecan as the primary therapy in the salvage setting, it was possible to make use of the patient response data to develop a gene expression signature predicting topotecan response. In contrast, our ability to do the equivalent for other used salvage agents is limited by the availability of patient samples. Clearly, this is a critical limitation since the goal is to predict sensitivity to a variety of potential agents to then select the most appropriate therapy for the individual patient. As an alternative approach, we have taken advantage of our recent work that has made use of assays in cancer cell lines to generate predictors of chemotherapy response, discussed in further detail in Example 5. In particular, we have made use of in vitro drug response data generated with the NCI-60 panel of cancer cell lines, coupled with Affymetrix gene expression data, to develop genomic predictors of response and resistance for a series of commonly used chemotherapeutic drugs. The predictor set for commonly used chemotherapeutics is disclosed in Table 5. The ability of these signatures to predict drug sensitivity has been validated in independent cell lines as well as patient samples.

We began with a proof of principle to ask if a predictor developed from cancer cell line assays for identifying response to topotecan could also predict response in the patient samples utilized in FIG. 10, using the patient samples as a validation/test set. As shown in FIG. 11A, this analysis revealed an accuracy of prediction of topotecan response in the patient samples (82%) that equaled that achieved with the patient-derived predictive model. Again, a test of statistical significance clearly demonstrated the ability of the signature to distinguish responder versus non-responder patients.

In addition to the validation of the topotecan predictor, we have also made use of small sets of samples from ovarian cancer patients treated with either docetaxel, adriamycin and taxol in the salvage setting. Again, the adriamycin, docetaxel and taxol signatures that were developed in the NCI-60 cell lines were used to predict the patient sample data. As shown in FIG. 11B, 11C both of these predictors were also capable of accurately predicting the response to the drugs in patient samples, achieving an accuracy in excess of 82% overall. Taken together, we conclude that it is possible to generate gene expression signatures that can predict with high accuracy the sensitivity to salvage chemotherapeutic drugs in ovarian cancer patients. The availability of predictors for these three agents, as well as the other predictors generated from the NCI-60 data, provides an opportunity to guide the selection of which drug would be optimally used for an individual patient. This is especially relevant given past studies that have not shown a clear superiority for either drug.

Patterns of Predicted Sensitivity to the Salvage Chemotherapy Drugs

To evaluate the potential for employing a battery of chemotherapy response predictors to guide decisions about salvage therapy, we examined the predicted sensitivity to various chemotherapies used in the salvage setting in a group of ovarian patients. Predictions are illustrated as a heatmap with red color indicating highest probability of response for the drug and blue color indicating lowest probability of response (FIG. 12A). It is evident from this analysis that while there are overlaps in the predicted sensitivities to the agents, there are also distinct groups of patients that are predicted to be sensitive to various single agent salvage agents. This is most clearly seen from the regression analyses depicted in FIG. 12B where it is clear that there is a strong inverse relationship between predicted topotecan sensitivity and sensitivity to either adriamycin, docetaxel, or etoposide. As such, this would provide an opportunity to direct the use of one or the other drugs based on the profile of the patient has the potential to achieve a better patient response.

In addition to the non-overlapping predicted sensitivities as illustrated above, there were also examples of overlap in the predicted sensitivity to the various agents. In particular, there was a significant predicted co-sensitivity between topotecan and taxol, again illustrated by a regression analysis as shown in FIG. 12C. Such a result might suggest the opportunity for the combination of topotecan and taxol, one not previously employed, to achieve a more effective therapeutic benefit.

Expanding Therapeutic Options for Advanced Stage Ovarian Cancer Patients

A series of gene expression profiles that predict salvage agent response, as detailed above and in Table 5, has the important potential to facilitate the identification of patients likely to benefit from various either single agent therapies or from novel combinations of agents. Nevertheless, it is also evident from the data in FIG. 12 that this will also identify patients resistant to both agents. Moreover, even those patients that initially respond to salvage therapies like topotecan or adriamycin are likely to eventually suffer a relapse. In either case, additional therapeutic options are needed.

In an effort to identify therapeutic options for topotecan or adriamycin resistant patients, we have used the development of gene expression profiles (or signatures) that reflect the activation status of several oncogenic pathways. We have applied these signatures to evaluate the status of pathways in the primary ovarian cancer samples. This approach provides a prediction of the relative probability of pathway deregulation of each of the primary ovarian cancers based on previously developed signatures.

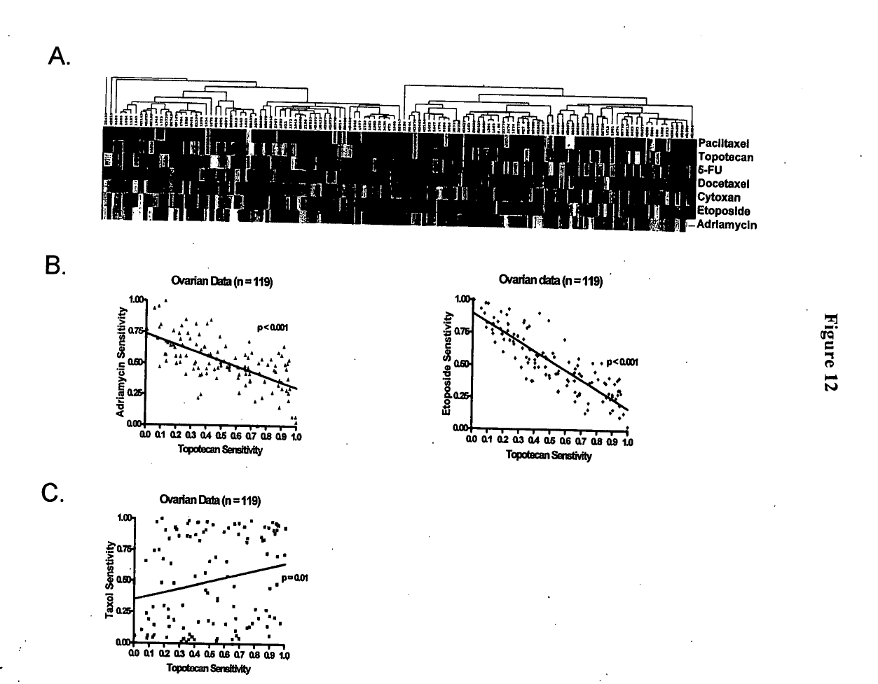

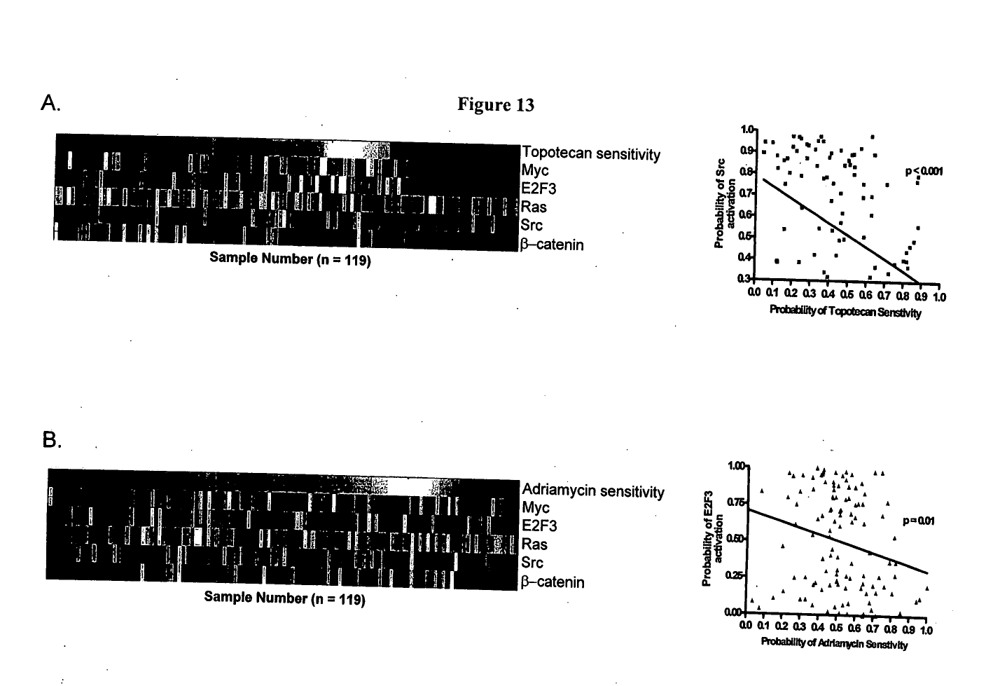

To illustrate the potential opportunity, we first stratified the patient samples based on predicted topotecan response to then determine if there were characteristic patterns of pathway deregulation associated with topotecan sensitivity or resistance. As shown in FIG. 13A, this analysis revealed a significant relationship between Src pathway deregulation and topotecan resistance. A similar analysis in the context of predicted adriamycin sensitivity revealed a significant relationship between deregulation of the E2F pathway and predicted resistance to adriamycin (FIG. 13B).

The results shown in FIG. 13 suggest that topotecan or adriamycin resistant tumors exhibit characteristic pathway deregulation and thus might display a sensitivity to inhibitors that target these pathways, based on our recent observations of a correlation between pathway deregulation and targeted drug sensitivity. To evaluate this possibility, we first examined the predicted relationships between topotecan sensitivity/resistance and predicted deregulation of Src pathway in a collection of 12 ovarian cancer cell lines. As shown in FIG. 14A, the predicted topotecan resistance in these cells is again associated with Src pathway deregulation. In parallel with the determination of pathway status in primary tumor specimens, these 12 ovarian cancer cell lines were subjected to assays for sensitivity to a Src-specific inhibitor (SU6656), both in single agent and combination with topotecan, using standard measures of cell proliferation. In each case, the measure of sensitivity to the drug was an effect on cell proliferation. The results of these assays clearly demonstrate a relationship between predicted topotecan resistance and sensitivity to the Src drug (FIG. 14B).

To explore a potential link between adriamycin resistance and deregulation of the E2F pathway, we have made use of the cdk inhibitor R-Roscovitine. Cyclin-dependent kinases (cdk), particularly cdk2 and cdk4, are critical regulatory activities controlling function of the retinoblastoma (Rb) protein which in turn, directly regulates E2F activity. As such, one might predict that deregulation of E2F pathway activity would also be linked with sensitivity to Roscovitine. Once again, the relationship between adriamycin resistance and E2F pathway deregulation that was seen in the ovarian tumors is also observed in the ovarian cancer cell lines (FIG. 14C). It is also clear that the predicted resistance to adriamycin coincides with sensitivity to R-Roscovitine (FIG. 14D).

Discussion

The challenge of cancer therapy is the ability to match the right drug with the right patient so as to achieve optimal therapeutic benefit and decrease toxicity related to empiric therapy. The availability of biomarkers of chemotherapy response is very limited such that overall response rate to treatment for recurrent disease are poor. In addition, it is also clear that the capacity of any one therapeutic agent to achieve success is likely low given the complexity of the oncogenic process that involves the accumulation of a large number of alterations, particularly in the context of advanced stage and recurrent disease. In light of this, the ability to develop predictors of response, as well as an ability to develop strategies for generating the most effective combinations of drugs for an individual patient, is key to moving toward therapeutic success. The work we describe here is, we believe, a step in this direction. In particular, our ability to develop predictors for salvage therapy response, coupled with information that can direct the use of other agents in combination with the salvage therapy, represents an opportunity to begin to tailor the most effective therapy for the individual patient with ovarian cancer.

Up to 30% of patients with advanced stage epithelial ovarian cancer fail to achieve a complete response to primary platinum-based therapy, and the majority those that initially demonstrate a complete response ultimately experience recurrent disease. Often these patients remain on minimally active chemotherapy for much of the remainder of their lives. As such, many of the challenges that women with ovarian cancer face are related to the chemotherapeutics they receive. Current empiric-based treatment strategies result in patients with chemo-resistant disease receiving multiple cycles of toxic therapy without success, prior to initiation of therapy with other potentially more active agents, or enrolment in clinical trials of new therapies. Throughout treatment for ovarian cancer, prolongation of survival and the successful maintenance of quality of life remain important goals, and improving our ability to manage the disease by optimizing the use of existing drugs and/or developing new agents is essential. In view of this, it is important that the choice of chemotherapy be individualized to each patient to reduce the incidence and severity of toxicities that could not only potentially limit quality of life, but also the ability to tolerate further therapy. To this end, individualizing treatments by identifying patients who are most likely to respond to specific agents, will not only increase response rates to those agents, but also limit toxicity and therefore improve quality of life for patients with non-responsive disease.

We believe the ability to accurately identify those patients likely to respond to single-agent salvage chemotherapies is a positive step towards the successful clinical application of predictive profiles. Currently, patients may receive multiple cycles of these salvage therapies before it becomes clear that they are not responding. These patients may experience detriment to bone marrow reserve, quality of life and a delay in timely initiation of alternate therapies, which include doxorubicin, gemcitabine, cyclophosphamide and oral etoposide, or enrolled in clinical trials. Nevertheless, the ability to identify those patients likely to respond to commonly used salvage chemotherapies is only one step in the path of achieving truly personalized medicine for cancer care, with the ultimate goal being effective cure of the disease. The capacity to identify additional therapeutic options, both for the patient predicted to be resistant to these salvage agents, but also to provide opportunities for combination therapy that might be more effective than single agent therapy, is clearly critical to achieving a successful strategy for treatment of the advanced stage ovarian cancer patient.

A potential limitation of the analysis we have described lies in the fact that primary tumor samples were used for gene expression measurements, prior to the initiation of adjuvant platinum/taxane and other salvage therapies. It might be argued that by the time salvage therapy was to be initiated substantial genetic alterations have occurred rendering the cells quite different from the primary resected tumor such that predictions based on gene expression profiles from primary specimen are unlikely to be accurate. The data we present does not support this position. While the genetic changes that occur with treatment and recurrence undoubtedly impact the overall genotype and phenotype, it is likely that many of the fundamental alterations that exist in the primary tumor are not only detectable at time of initial diagnosis but may also drive the response of clonally expanded recurrences to salvage therapy. Our preliminary predictive profiles and the analysis of oncogenic pathway deregulation in cell lines support this premise. Although gene expression profiles of recurrent ovarian cancer biopsy specimens prior to the initiation of each salvage therapy would likely provide additional information, such specimens are not routinely obtained and access to them cannot be relied upon for clinical or research purposes.

We suggest a next step in the path towards more effective and ultimately personal treatment is an ability to identify combinations of therapeutic agents that might best match characteristics of the individual patient. We believe the ability to make use of multiple forms of genomic information, both measures of pathway deregulation as well as signatures developed to predict sensitivity to cytotoxic chemotherapy drugs, provides such an opportunity (FIG. 15). Of course, this is only a proposal and must await prospective clinical studies that can evaluate the efficacy of such treatment strategies. Nevertheless, we suggest that the importance of this approach is also an ability to identify potential such therapeutic opportunities that in fact can then be tested in such trials. As such, response rates can be improved, non-active toxic agents avoided, bone marrow spared, and quality of life enhanced. Ultimately, defining the biologic underpinnings of response to therapy will facilitate the development of more active agents that may improve survival for women with ovarian cancer.

### Example 4

### Gene Expression Profiles for Predicting Response to Chemotherapy for Advanced Stage Ovarian Cancer

The purpose of this experiment is to validate the ability of expression profiles to predict response to chemotherapy for advanced stage epithelial ovarian cancer, by analysis of primary ovarian cancer and also cells obtained from ascites. These profiles can be obtained by analysis of the primary ovarian cancer and also from ovarian cancer cells retrieved from ascites.

Methods and Procedures

We validate our ability to predict response to adjuvant chemotherapy for advanced stage ovarian cancer by using microarray expression analysis of primary ovarian cancers and cytologic ascites specimens. This also validates expression patterns as predictors of response to salvage therapies in patients who experience persistent or recurrent disease.

Following IRB-approved informed consent, ovarian cancer and ascites specimens are obtained from patients undergoing primary surgical cytoreduction at the H. Lee Moffitt Cancer Center and Research Institute. In addition to ovarian tissue, approximately 300 cc of ascites is collected. Microarray analysis is applied to a series of approximately 60 advanced stage epithelial ovarian cancers and a subset of 20 cytologic (ascites) specimens. For each ascites specimen, a cell count is obtained. For ascites specimens, where necessary, the Arcturus RiboAmp OA Kit that is optimized for amplification of RNA for use with oligonucleotide arrays is used to amplify sufficient quantities of RNA for use in array analysis. Following array analysis, for primary ovarian cancers and ascites specimens, gene expression profiles are interrogated using the statistical predictive model described herein.

Following microarray analysis of resected cancer specimen, patients are classified as “platinum-sensitive” or “platinum-resistant” according to the predictive model, and followed using standard medical protocols (e.g., using clinical exam, CA125, and radiographic imaging, where indicated). At completion of 6 cycles of adjuvant platinum-based chemotherapy, patients are evaluated for response and categorized as “platinum-sensitive” or “platinum-resistant,” as measured by established clinical parameters. Response criteria for patients with measurable disease are based upon WHO guidelines (Miller et al., Cancer 1981; 47:207-14). CA-125 is used to classify responses only in the absence of a measurable lesion; CA-125 response criteria is based on established guidelines (Rustin et al., J. Clin. Oncol. 1996;14: 1545-51, Rustin et al., Ann. Oncol. 1999; 10). A complete response (“platinum-sensitive”) is defined as a complete disappearance of all measurable and assessable disease or, in the absence of measurable lesions, a normalization of the CA-125 level following 3 cycles of adjuvant therapy. “Platinum resistant” is classified as patients who demonstrate only a partial response, have no response, or progress during adjuvant therapy. A partial response is considered a 50% or greater reduction in the product obtained from measurement of each bi-dimensional lesion for at least 4 weeks or a drop in the CA-125 by at least 50% for at least 4 weeks. Disease progression is defined as a 50% or greater increase in the product from any lesion documented within 8 weeks of study entry, the appearance of any new lesion within 8 weeks of entry onto study, or any increase in the CA-125 from baseline at study entry. Stable disease is defined as disease not meeting any of the above criteria. The clinical response is then compared to the response predicted by expression profile. Predictive values of the expression profile is then calculated.

Microarray Analysis Methodology—We analyze 22,000 well-substantiated human genes using the Affymetrix Human U133A GeneChip. Total RNA and the target probes are prepared, hybridized, washed and scanned according to the manufacturer's instructions. The average difference measurements computed in the Affymetrix Microarray Analysis Suite (v.5.0) serve as a relative indicator of the level of expression. Expression profiles are compared between samples from women who did, and did not, exhibit a response to chemotherapy. Gene expression profiles are interrogated using our predictive tool.

Microarray statistical analysis—In addition to application of our statistical predictive model to ovarian cancers, we also seek to further improve the model. Ongoing analysis is performed using predictive statistical tree models. Large numbers of clusters are used to generate a corresponding number of metagene patterns. These metagenes are then subjected to formal predictive analysis in a Bayesian classification tree analysis. Overall predictions for an individual sample will be generated by averaging predictions. We perform iterative leave-out-one-sample cross-validation predictions, which involves leaving each tumor out of the data set one at a time and then refitting the model from the remaining tumors and predicting the hold-out case. This rigorously tests and improves the predictive value of the model with each additional collected case.

Gene expression profiles are also analyzed on the basis of response to salvage therapies. Patients with persistent or recurrent disease are followed through their salvage chemotherapy and their response evaluated and compared to the gene expression profile predicted response. In this subset of patients, expression profiles from primary specimens are evaluated to identify gene expression patterns associated with, and predictive of, response to individual salvage therapies. Ability to predict response to salvage therapy is thus evaluated.

Ethical Considerations—Patients undergo pre-operative informed consent prior to any intra-operative cancer specimen being collected for analysis. Confidentiality is maintained to avoid, whenever possible, the risk for discrimination towards the individual. All information relating to the patient's participation in this study is kept strictly confidential. DNA and tumor tissue samples are identified by a code number and all other identifying information are removed when the specimen arrives in the tumor bank following collection. The patient is informed that she will not be contacted regarding research findings from analysis done using the samples due to the preliminary nature of this type of research. Necessary data is abstracted from the patient's hospital records. The patients are not contacted. Patients are assigned unique identifiers separate from their hospital record numbers and the working database contains only the unique identifier. This study validates the concept of using gene expression profiles to predict response to chemotherapy. The results of this study are not expected to have implication for the treatment of the individual subjects.

Statistical considerations and Endpoints—To date, no reliable statistical technique exists for power analysis and sample-size calculations for microarray studies. Based on our experience with array studies and the development of the predictive model from analysis of 32 advanced ovarian cancers, we have chosen a sample size of approximately 60 prospectively collected cancers in an effort to further validate our model. Gene expression profiles are analyzed and compared to our predictive statistical model. Samples are classified as either platinum-responders or non-responders. The patient is followed and their response to platinum therapy is recorded. Predicted response and actual response are compared and the positive and negative predictive values of the model are determined. The study endpoint is the completion of array analysis, as well as predicted and clinical categorization of all 60 patients as platinum-responders or non-responders.

### Example 5

### A Gene Expression Based Predictor of Sensitivity to Docetaxel

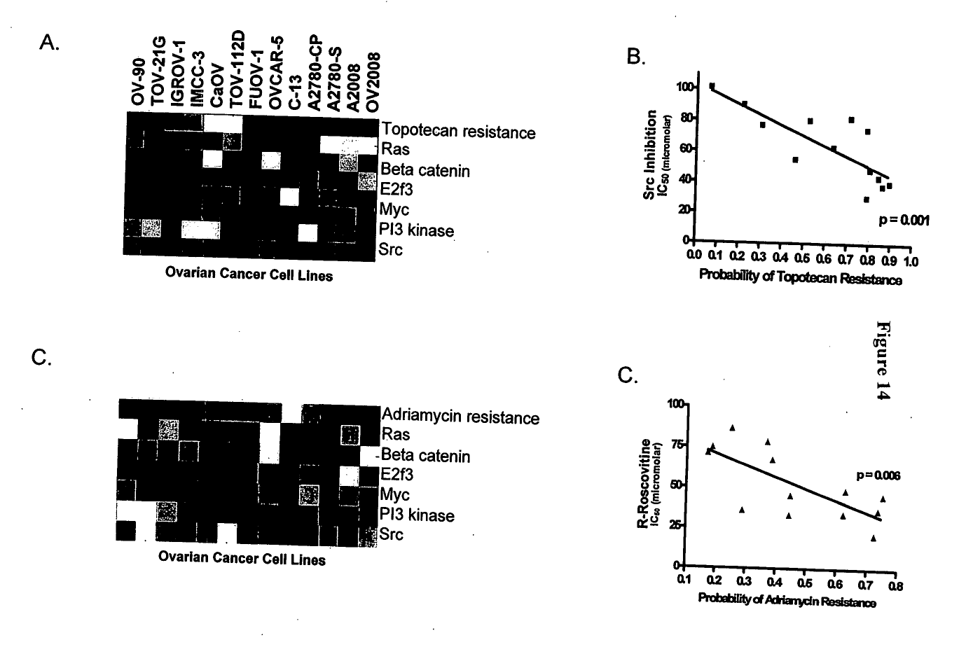

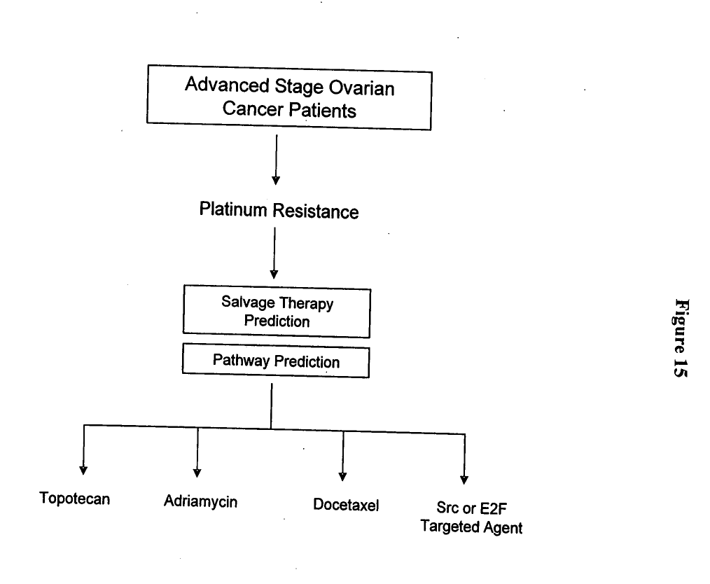

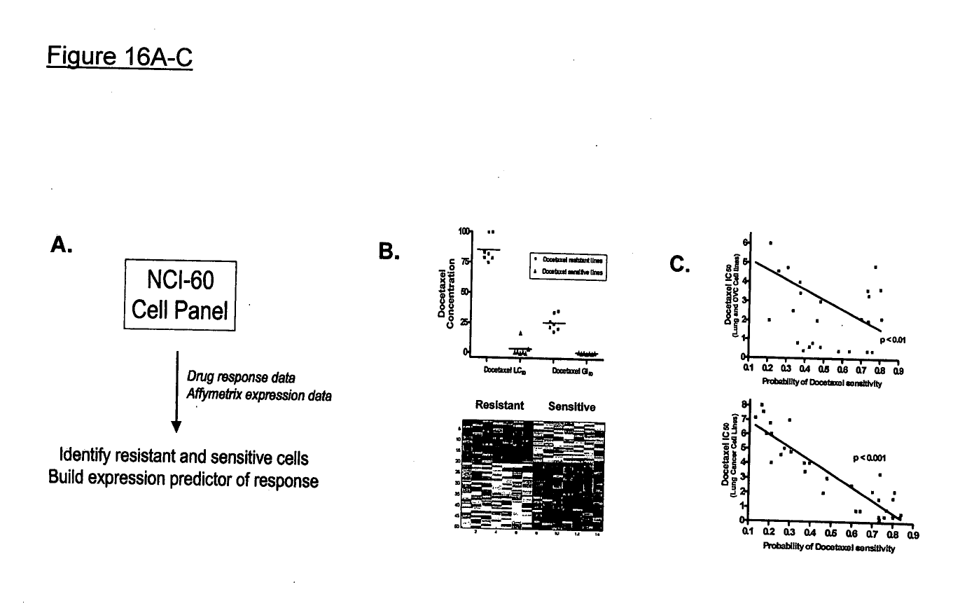

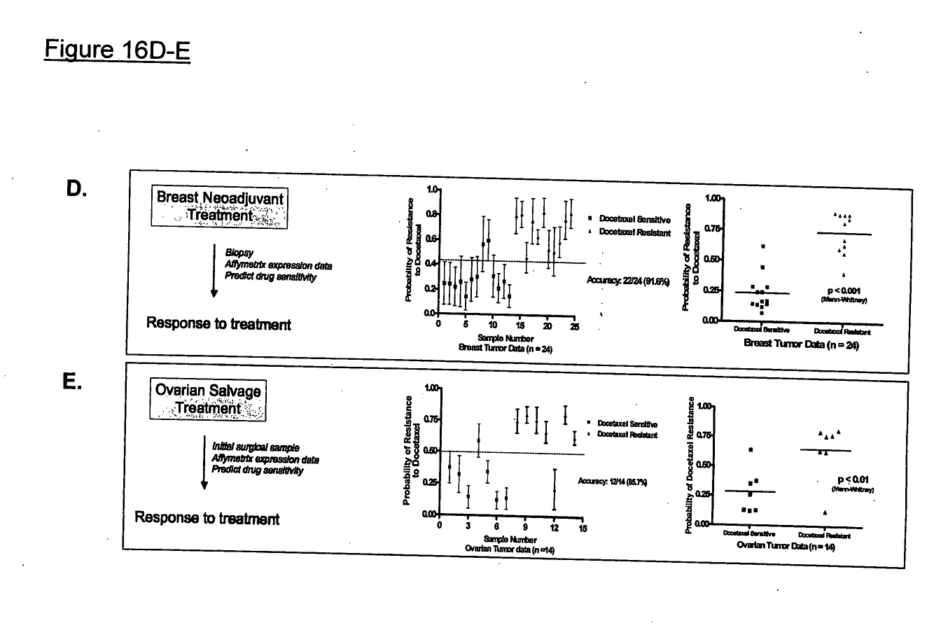

To develop predictors of cytotoxic chemotherapeutic drug response, we used an approach similar to previous work analyzing the NCI-60 panel,⁴⁹ first identifying cell lines that were most resistant or sensitive to docetaxel (FIG. 16A, B) and then genes whose expression most highly correlated with drug sensitivity, using Bayesian binary regression analysis to develop a model that differentiates a pattern of docetaxel sensitivity from resistance. A gene expression signature consisting of 50 genes was identified that classified on the basis of docetaxel sensitivity (FIG. 16B, bottom panel).

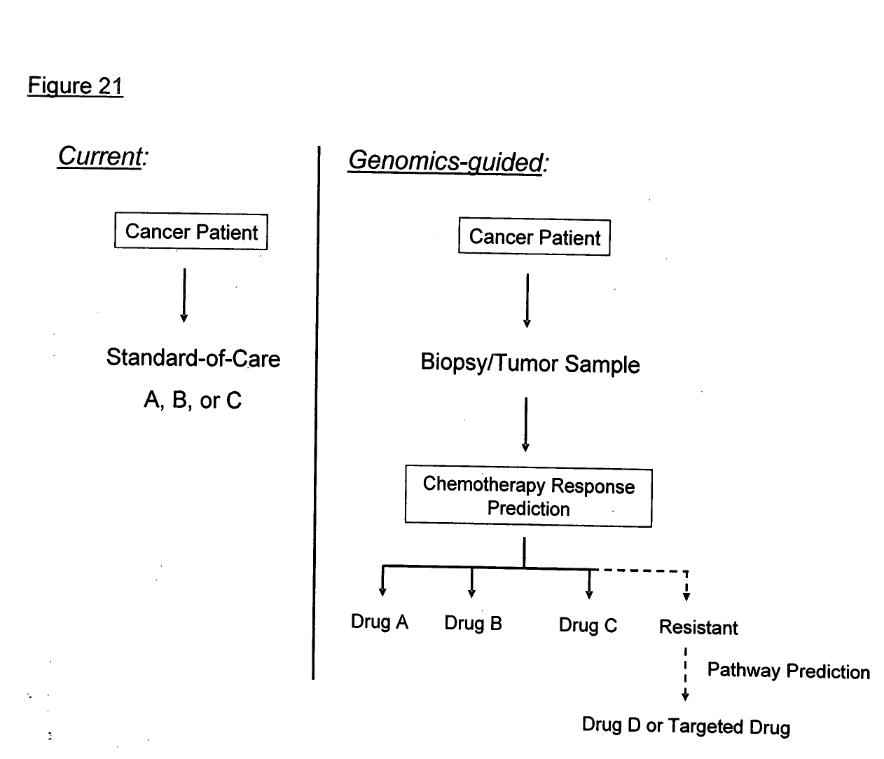

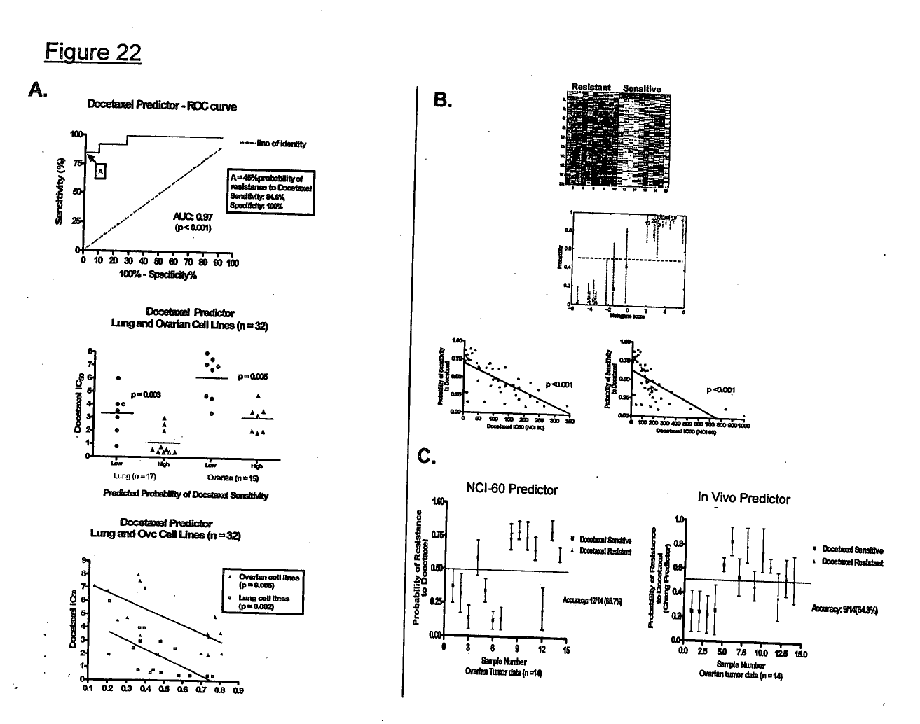

In addition to leave-one-out cross validation, we utilized an independent dataset derived from docetaxel sensitivity assays in a series of 30 lung and ovarian cancer cell lines for further validation. As shown in FIG. 16C (top panel), the correlation between the predicted probability of sensitivity to docetaxel (in both lung and ovarian cell lines) and the respective IC50 for docetaxel confirmed the capacity of the docetaxel predictor to predict sensitivity to the drug in cancer cell lines (FIG. 22). In each case, the accuracy exceeded 80%. Finally, we made use of a second independent dataset that measured docetaxel sensitivity in a series of 29 lung cancer cell lines (Gemma A, GEO accession number: GSE 4127). As shown in FIG. 16C (bottom panel), the docetaxel sensitivity model developed from the NCI-60 panel again predicted sensitivity in this independent dataset, again with an accuracy exceeding 80%.

Utilization of the Expression Signature to Predict Docetaxel Response in Patients

The development of a gene expression signature capable of predicting in vitro docetaxel sensitivity provides a tool that might be useful in predicting response to the drug in patients. We have made use of published studies with clinical and genomic data that linked gene expression data with clinical response to docetaxel in a breast cancer neoadjuvant study⁵⁰ (FIG. 16D) to test the capacity of the in vitro docetaxel sensitivity predictor to accurately identify those patients that responded to docetaxel. Using a 0.45 predicted probability of response as the cut-off for predicting positive response, as determined by ROC curve analysis (FIG. 22A), the in vitro generated profile correctly predicted docetaxel response in 22 out of 24 patient samples, achieving an overall accuracy of 91.6% (FIG. 16D). Applying a Mann-Whitney U test for statistical significance demonstrates the capacity of the predictor to distinguish resistant from sensitive patients (FIG. 16D, right panel). We extended this further by predicting the response to docetaxel as salvage therapy for ovarian cancer. As shown in FIG. 16E, the prediction of response to docetaxel in patients with advanced ovarian cancer achieved an accuracy exceeding 85% (FIG. 16E, middle panel). Further, an analysis of statistical significance demonstrated the capacity of the predictors to distinguish patients with resistant versus sensitive disease (FIG. 16E, right panel).

We also performed a complementary analysis using the patient response data to generate a predictor and found that the in vivo generated signature of response predicted sensitivity of NCI-60 cell lines to docetaxel (FIG. 22B). This crossover is further emphasized by the fact that the genes represented in either the initial in vitro generated docetaxel predictor or the alternative in vivo predictor exhibit considerable overlap. Importantly, both predictors link to expected targets for docetaxel including bcl-2, TRAG, erb-B2, and tubulin genes, all previously described to be involved in taxane chemoresistance⁵¹⁻⁵⁴ (Table 5). We also note that the predictor of docetaxel sensitivity developed from the NCI-60 data was more accurate in predicting patient response in the ovarian samples than the predictor developed from the breast neoadjuvant patient data (85.7% vs. 64.3%) (FIG. 22C).

Development of a Panel of Gene Expression Signatures that Predict Sensitivity to Chemotherapeutic Drugs

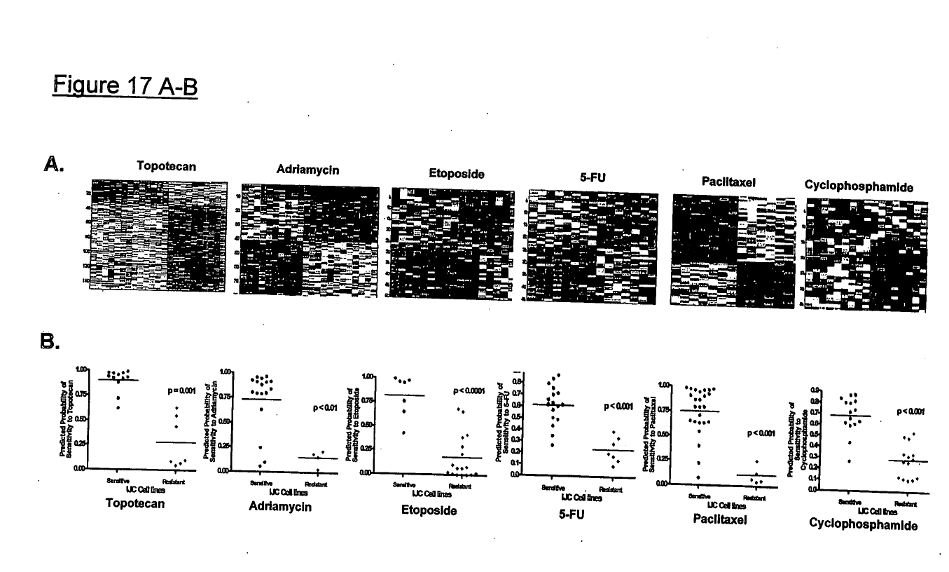

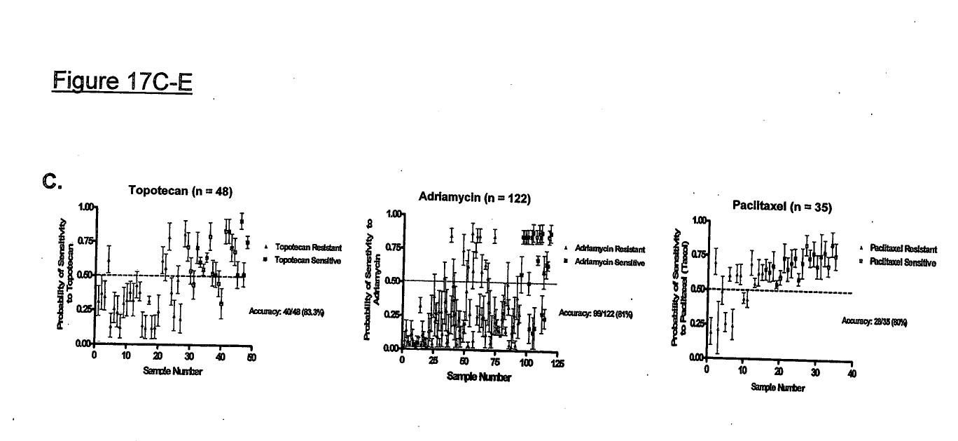

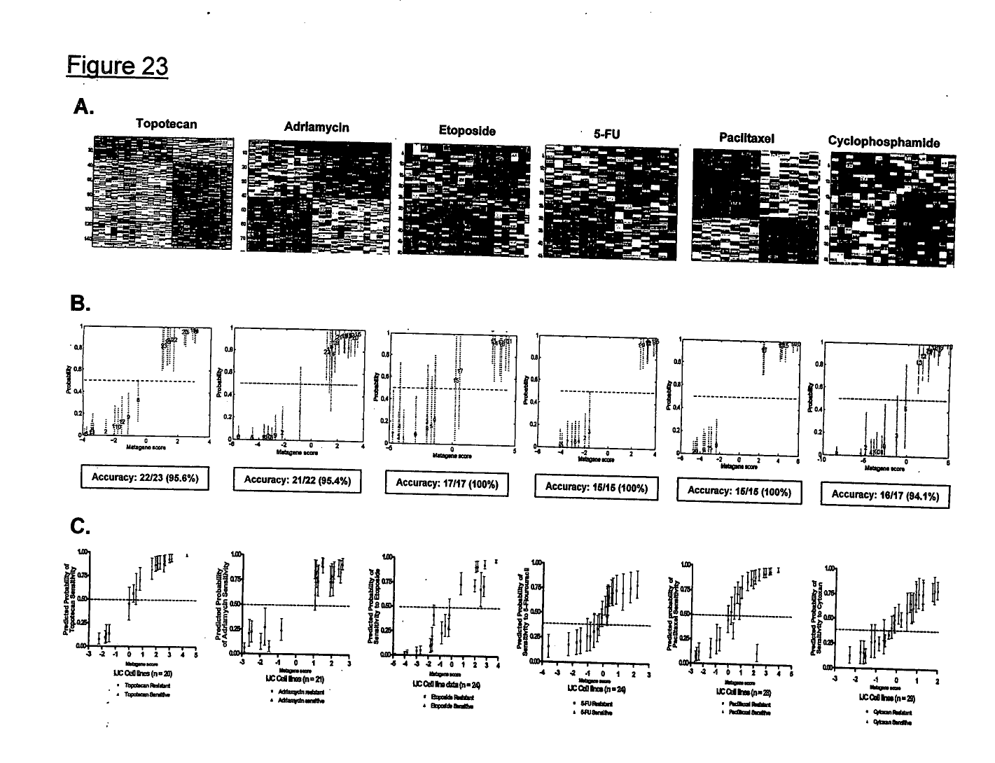

Given the development of a docetaxel response predictor, we have examined the NCI-60 dataset for other opportunities to develop predictors of chemotherapy response. Shown in FIG. 17A are a series of expression profiles developed from the NCI-60 dataset that predict response to topotecan, adriamycin, etoposide, 5-flourouracil (5-FU), paclitaxel, and cyclophosphamide. In each case, the leave-one-out cross validation analyses demonstrate a capacity of these profiles to accurately predict the samples utilized in the development of the predictor (FIG. 23, middle panel). Each profile was then further validated using in vitro response data from independent datasets; in each case, the profile developed from the NCI-60 data was capable of accurately (>85%) predicting response in the separate dataset of approximately 30 cancer cell lines for which the dose response information and relevant Affymetrix U133A gene expression data is publicly available³⁷ (FIG. 23 (bottom panel) and Table 6). Once again, applying a Mann-Whitney U test for statistical significance demonstrates the capacity of the predictor to distinguish resistant from sensitive patients (FIG. 17B).

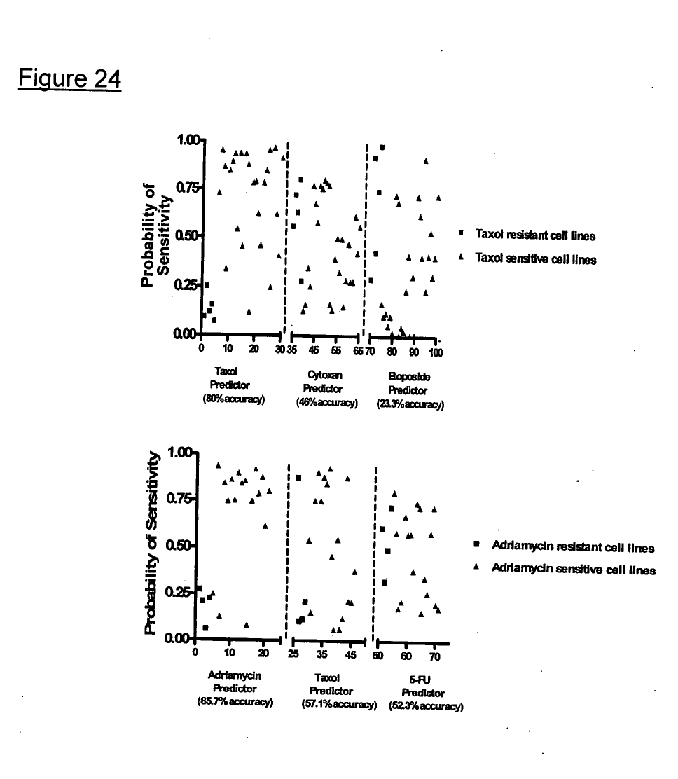

In addition to the capacity of each signature to distinguish cells that are sensitive or resistant to a particular drug, we also evaluated the extent to which a signature was also specific for an individual chemotherapeutic agent. From the example shown in FIG. 24, using the validations of chemosensitivity seen in the independent European (IJC) cell line data it is clear that each of the signatures is specific for the drug that was used to develop the predictor. In each case, individual predictors of response to the various cytotoxic drugs was plotted against cell lines known to be sensitive or resistant to a given chemotherapeutic agent (e.g., adriamycin, paclitaxel).

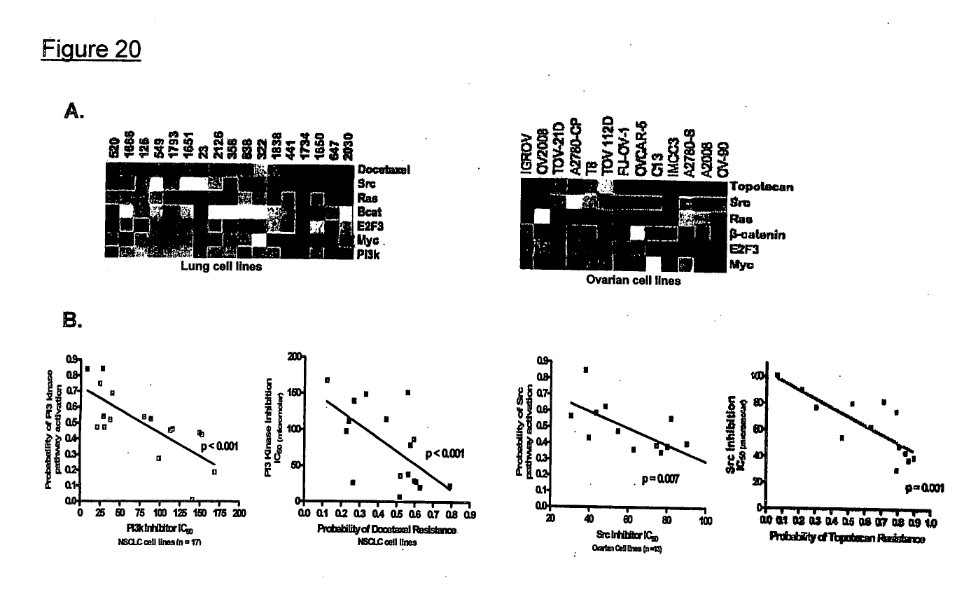

Given the ability of the in vitro developed gene expression profiles to predict response to docetaxel in the clinical samples, we extended this approach to test the ability of additional signatures to predict response to commonly used salvage therapies for ovarian cancer and an independent dataset of samples from adriamycin treated patients (Evans W, GSE650, GSE651). As shown in FIG. 20C, each of these predictors was capable of accurately predicting the response to the drugs in patient samples, achieving an accuracy in excess of 81% overall. In each case, the positive and negative predictive values confirm the validity and clinical utility of the approach (Table 6).

Chemotherapy Response Signatures Predict Response to Multi-drug Regimens

Many therapeutic regimens make use of combinations of chemotherapeutic drugs raising the question as to the extent to which the signatures of individual therapeutic response will also predict response to a combination of agents. To address this question, we have made use of data from a breast neoadjuvant treatment that involved the use of paclitaxel, 5-flourouracil, adriamycin, and cyclophosphamide (TFAC)^{55,56} (FIG. 18A). Using available data from the 51 patients to then predict response with each of the single agent signatures (paclitaxel, 5-FU, adriamycin and cyclophosphamide) developed from the NCI-60 cell line analysis; we then compared to the clinical outcome information which was represented as complete pathologic response. As shown in FIG. 18A (middle panel), the predicted response based on each of the individual chemosensitivity signatures indicated a significant distinction between the responders (n=13) and non-responders (n=38) with the exception of 5-flourouracil. Importantly, the combined probability of sensitivity to the four agents in this TFAC neoadjuvant regimen was calculated using the probability theorem and it is clear from this analysis that the prediction of response based on a combined probability of sensitivity, built from the individual chemosensitivity predictions yielded a statistically significant (p<0.0001, Mann Whitney U) distinction between the responders and non-responders (FIG. 18A, right panel).

As a further validation of the capacity to predict response to combination therapy, we have made use of gene expression data generated from a collection of breast cancer (n=45) samples from patients who received 5-flourouracil, adriamycin and cyclophosphamide (FAC) in the adjuvant chemotherapy set. As shown in FIG. 18B (left panel), the predicted response based on signatures for 5-FU, adriamycin, and cyclophosphamide indicated a significant distinction between the responders (n=34) and non-responders (n=11) for each of the single agent predictors. Furthermore, the combined probability of sensitivity to the three agents in the FAC regimen was calculated and shown in the middle panel of FIG. 18B. It is evident from this analysis that the prediction of response based on a combined probability of sensitivity to the FAC regimen yielded a clear, significant (p<0.001, Mann Whitney U) distinction between the responders and non-responders (accuracy: 82.2%, positive predictive value: 90.3%, negative predictive value: 64.3%). We note that while it is difficult to interpret the prediction of clinical response in the adjuvant setting since many of these patients were likely free of disease following surgery, the accurate identification of non-responders is a clear endpoint that does confirm the capacity of the signatures to predict clinical response.

As a further measure of the relevance of the predictions, we examined the prognostic significance of the ability to predict response to FAC. As shown in FIG. 18B (right panel), there was a clear distinction in the population of patients identified as sensitive or resistant to FAC, as measured by disease-free survival. These results, taken together with the accuracy of prediction of response in the neoadjuvant setting where clinical endpoints are uncomplicated by confounding variables such as prior surgery, and results of the single agent validations, leads us to conclude that the signatures of chemosensitivity generated from the NCI-60 panel do indeed have the capacity to predict therapeutic response in patients receiving either single agent or combination chemotherapy (Table 7).

When comparing individual genes that constitute the predictors, it was interesting to observe that the gene coding for MAP-Tau, described previously as a determinant of paclitaxel sensitivity,⁵⁶ was also identified as a discriminator gene in the paclitaxel predictor generated using the NCI-60 data. Although, similar to the docetaxel example described earlier, a predictor for TFAC chemotherapy developed using the NCI-60 data was superior to the ability of the MAP-Tau based predictor described by Pusztai et al (Table 8). Similarly, p53, methyltetrahydrofolate reductase gene and DNA repair genes constitute the 5-flourouracil predictor, and excision repair mechanism genes (e.g., ERCC4), retinoblastoma pathway genes, and bcl-2 constitute the adriamycin predictor, consistent with previous reports (Table 5).

Patterns of Predicted Chemotherapy Response Across a Spectrum of Tumors

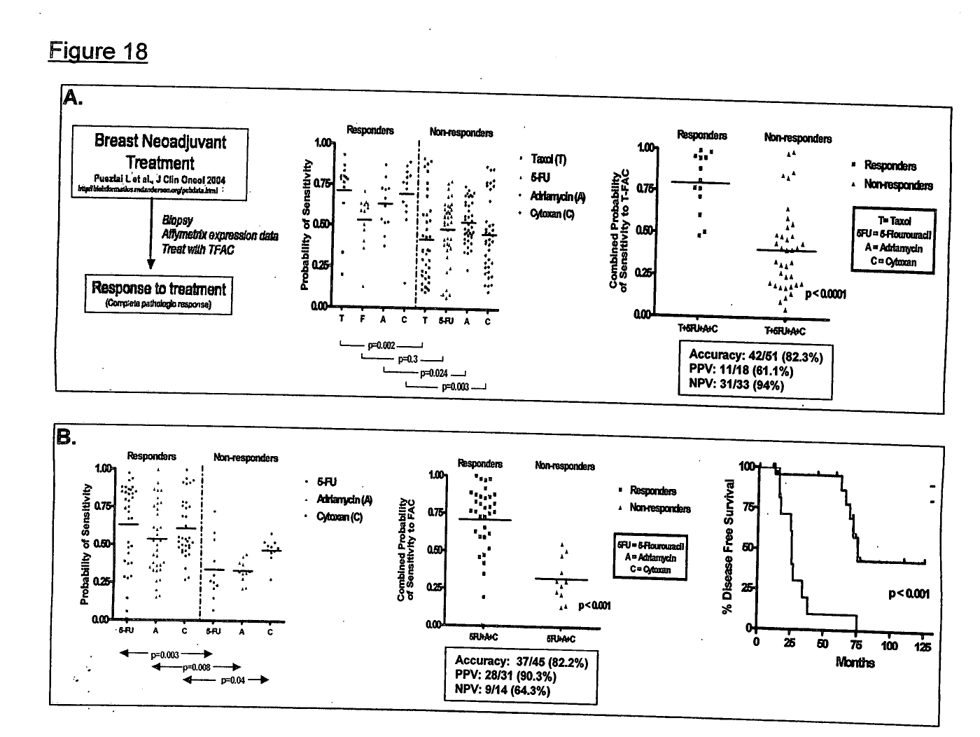

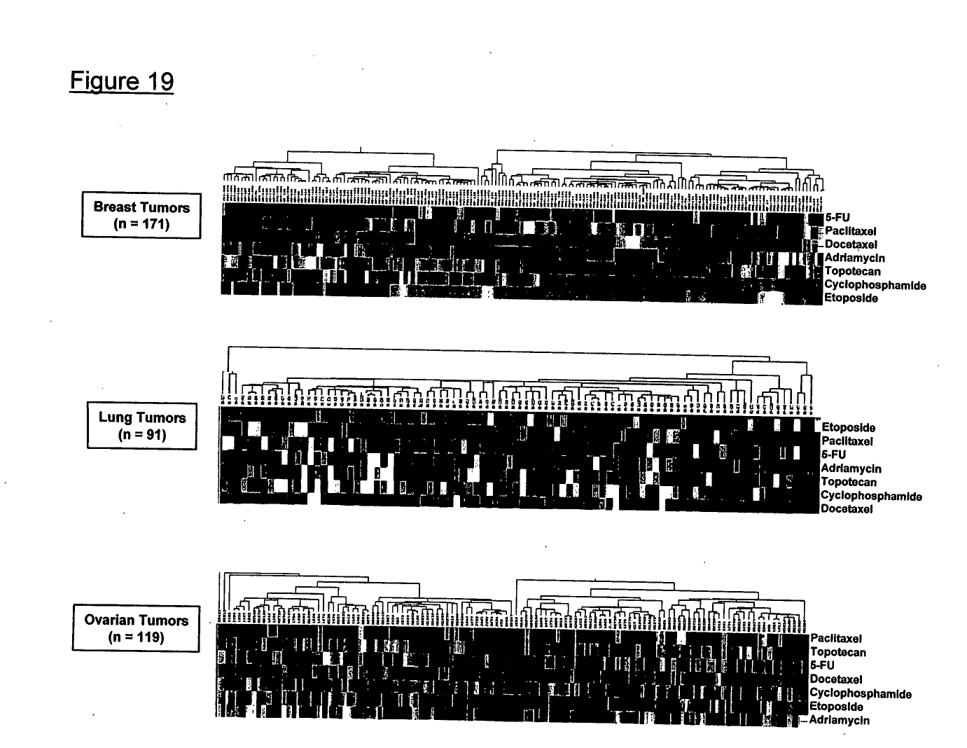

The availability of genomic-based predictors of chemotherapy response could potentially provide an opportunity for a rational approach to selection of drugs and combination of drugs. With this in mind, we have utilized the panel of chemotherapy response predictors described in FIG. 21 to profile the potential options for use of these agents, by predicting the likelihood of sensitivity to the seven agents in a large collection of breast, lung, and ovarian tumor samples. We then clustered the samples according to patterns of predicted sensitivity to the various chemotherapeutics, and plotted a heatmap in which high probability of sensitivity/response is indicated by red and low probability or resistance is indicated by blue (FIG. 19).

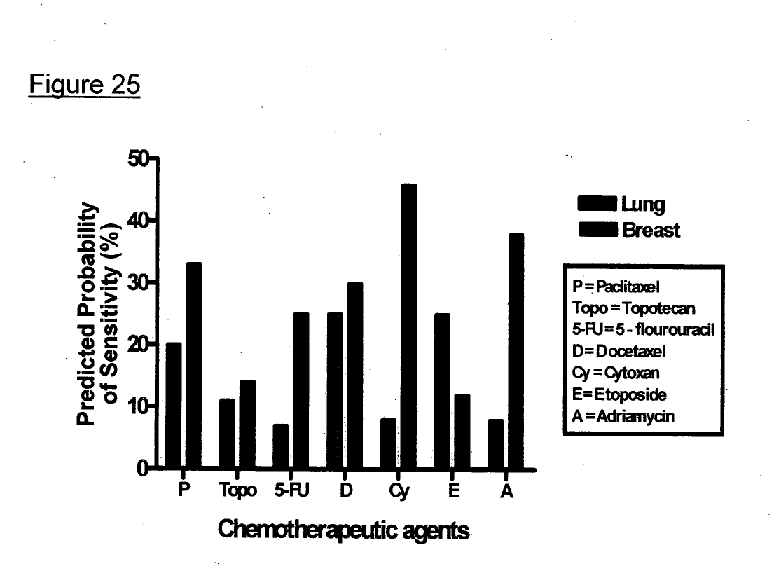

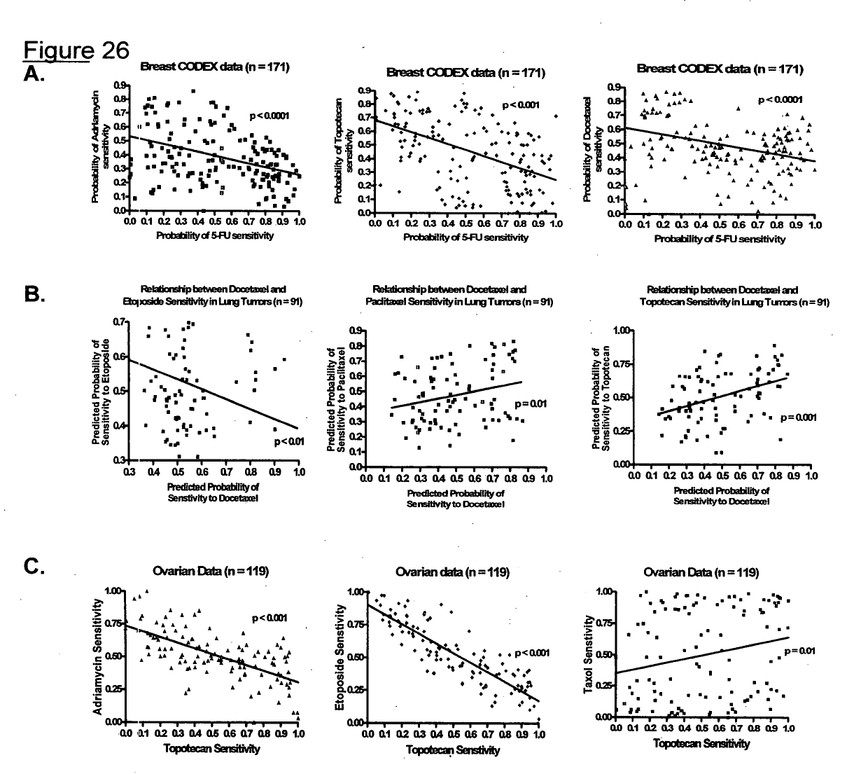

As shown in FIG. 18, there are clearly evident patterns of predicted sensitivity to the various agents. In many cases, the predicted sensitivities to the chemotherapeutic agents are consistent with the previously documented efficacy of single agent chemotherapies in the individual tumor types⁵⁷. For instance, the predicted response rate for etoposide, adriamycin, cyclophosphamide, and 5-FU approximate the observed response for these single agents in breast cancer patients (FIG. 25). Likewise, the predicted sensitivity to etoposide, docetaxel, and paclitaxel approximates the observed response for these single agents in lung cancer patients (FIG. 25). This analysis also suggests possibilities for alternate treatments. As an example, it would appear that breast cancer patients likely to respond to 5-flourouracil are resistant to adriamycin and docetaxel (FIG. 26A). Likewise, in lung cancer, docetaxel sensitive populations are likely to be resistant to etoposide (FIG. 26B). This is a potentially useful observation considering that both etoposide and docetaxel are viable front-line options (in conjunction with cis/carboplatin) for patients with lung cancer.⁵⁸ A similar relationship is seen between topotecan and adriamycin, both agents used in salvage chemotherapy for ovarian cancer (FIG. 26C). Thus, by identifying patients/patient cohorts resistant to certain standard of care agents, one could avoid the side effects of that agent (e.g. topotecan) without compromising patient outcome, by choosing an alternative standard of care (e.g., adriamycin).

Linking Predictions of Chemotherapy Sensitivity to Oncogenic Pathway Deregulation

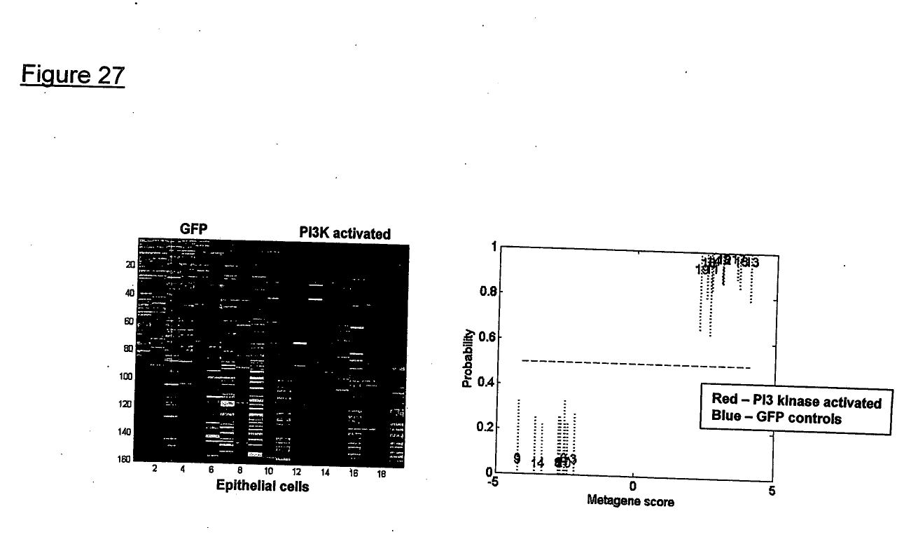

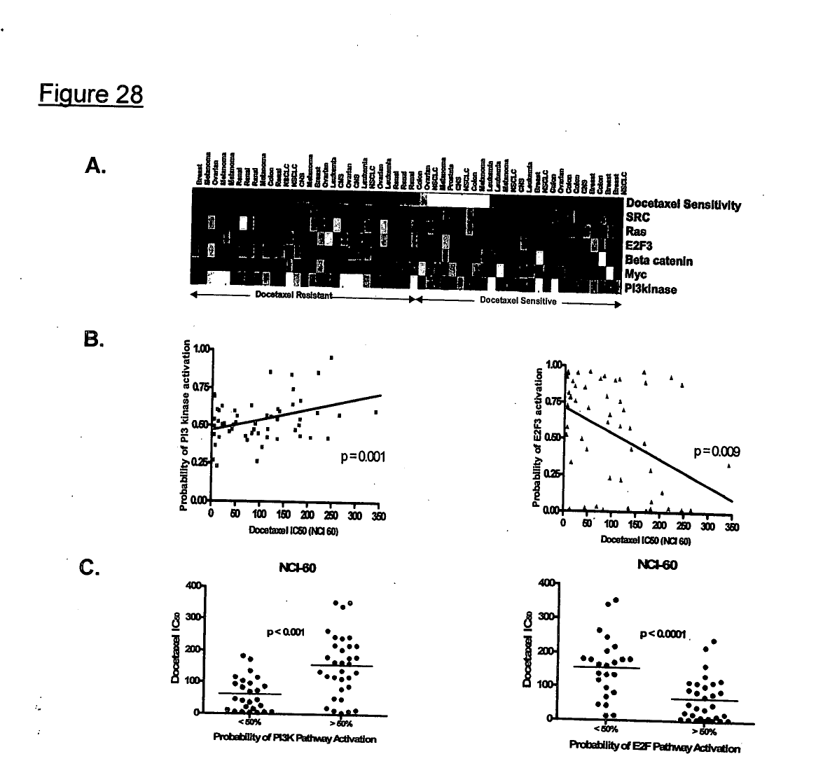

Most patients who are resistant to chemotherapeutic agents are then recruited into a second or third line therapy or enrolled to a clinical trial.^{38,59} Moreover, even those patients who initially respond to a given agent are likely to eventually suffer a relapse and in either case, additional therapeutic options are needed. As one approach to identifying such options, we have taken advantage of our recent work that describes the development of gene expression signatures that reflect the activation of several oncogenic pathways.³⁶ To illustrate the approach, we first stratified the NCI cell lines based on predicted docetaxel response and then examined the patterns of pathway deregulation associated with docetaxel sensitivity or resistance (FIG. 28A). Regression analysis revealed a significant relationship between PI3 kinase pathway deregulation and docetaxel resistance, as seen by the linear relationship (p=0.001) between the probability of PI3 kinase activation and the IC50 of docetaxel in the cell lines (FIG. 27, 28B, and Table 9).

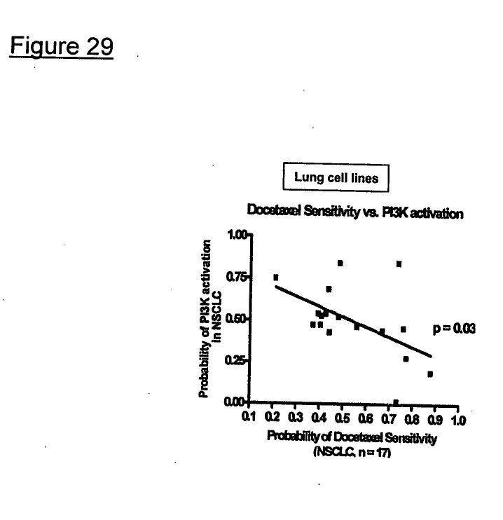

The results linking docetaxel resistance with deregulation of the PI3 kinase pathway, suggests an opportunity to employ a PI3 kinase inhibitor in this subgroup, given our recent observations that have demonstrated a linear positive correlation between the probability of pathway deregulation and targeted drug sensitivity.³⁶ To address this directly, we predicted docetaxel sensitivity and probability of oncogenic pathway deregulation using DNA microarray data from 17 NSCLC cell lines (FIG. 20A, left panel). Consistent with the analysis of the NCI-60 cell line panel, the cell lines predicted to be resistant to docetaxel were also predicted to exhibit PI3 kinase pathway activation (p=0.03, log-rank test, FIG. 29). In parallel, the lung cancer cell lines were subjected to assays for sensitivity to a PI3 kinase specific inhibitor (LY-294002), using a standard measure of cell proliferation.^{36, 38, 59} As shown by the analysis in FIG. 20B (left panel), the cell lines showing an increased probability of PI3 kinase pathway activation were also more likely to respond to a PI3 kinase inhibitor (LY-294002)(p=0.001, log-rank test)). The same relationship held for prediction of resistance to docetaxel—these cells were more likely to be sensitive to PI3 kinase inhibition (p<0.001, log-rant test)(FIG. 20B, left panel).

An analysis of a panel of ovarian cancer cell lines provided a second example. Ovarian cell lines that are predicted to be topotecan resistant (FIG. 20A, right panel) have a higher likelihood of Src pathway deregulation and there is a significant linear relationship (p=0.001, log rank) between the probability of topotecan resistance and sensitivity to a drug that inhibits the Src pathway (SU6656)(FIG. 20B, right panel). The results of these assays clearly demonstrate an opportunity to potentially mitigate drug resistance (e.g., docetaxel or topotecan) using a specific pathway-targeted agent, based on a predictor developed from pathway deregulation (i.e., PI3 kinase or Src inhibition).

Taken together, these data demonstrate an approach to the identification of therapeutic options for chemotherapy resistant patients, as well as the identification of novel combinations for chemotherapy sensitive patients, and thus represents a potential strategy to a more effective treatment plan for cancer patients, after future prospective validations trials (FIG. 21).

Methods

NCI-60 data. The (−log 10(M)) GI50/IC50, TGI (Total Growth Inhibition dose) and LC50 (50% cytotoxic dose) data was used to populate a matrix with MATLAB software, with the relevant expression data for the individual cell lines. Where multiple entries for a drug screen existed (by NCS number), the entry with the largest number of replicates was included. Incomplete data were assigned as Nan (not a number) for statistical purposes. To develop an in vitro gene expression based predictor of sensitivity/resistance from the pharmacologic data used in the NCI-60 drug screen studies, we chose cell lines within the NCI-60 panel that would represent the extremes of sensitivity to a given chemotherapeutic agent (mean GI50+/−1SD). Relevant expression data (updated data available on the Affymetrix U95A2 GeneChip) for the solid tumor cell lines and the respective pharmacological data for the chemotherapeutics was downloaded from the NCI website (http://dtp.nci.nih.gov/docs/cancer/cancer_data.html). The individual drug sensitivity and resistance data from the selected solid tumor NCI-60 cell lines was then used in a supervised analysis using binary regression methodologies, as described previously,⁶⁰ to develop models predictive of chemotherapeutic response.

Human ovarian cancer samples. We measured expression of 22,283 genes in 13 ovarian cancer cell lines and 119 advanced (FIGO stage III/IV) serous epithelial ovarian carcinomas using Affymetrix U133A GeneChips. All ovarian cancers were obtained at initial cytoreductive surgery from patients. All tissues were collected under the auspices of respective institutional (Duke University Medical Center and H. Lee Moffitt Cancer Center) IRB approved protocols involving written informed consent.

Full details of the methods used for RNA extraction and development of gene expression signatures representing deregulation of oncogenic pathways in the tumor samples are recently described.³⁶ Response to therapy was evaluated using standard criteria for patients with measurable disease, based upon WHO guidelines.²⁸

Lung and ovarian cancer cell culture. Total RNA was extracted and oncogenic pathway predictions was performed similar to the methods described previously.³⁶

Cross-platform Affymetrix Gene Chip comparison. To map the probe sets across various generations of Affymetrix GeneChip arrays, we utilized an in-house program, Chip Comparer (http://tenero.duhs.duke.edu/genearray/perl/chip/chipcomparer.pl) as described previously.³⁶

Cell proliferation assays. Growth curves for cells were produced by plating 500-10,000 cells per well in 96-well plates. The growth of cells at 12 hr time points (from t=12 hrs) was determined using the CellTiter 96 Aqueous One 23 Solution Cell Proliferation Assay Kit by Promega, which is a colorimetric method for determining the number of growing cells.³⁶ The growth curves plot the growth rate of cells vs. each concentration of drug tested against individual cell lines. Cumulatively, these experiments determined the concentration of cells to use for each cell line, as well as the dosing range of the inhibitors. The final dose-response curves in our experiments plot the percent of cell population responding to the chemotherapy vs. the concentration of the drug for each cell line. Sensitivity to docetaxel and a phosphatidylinositol 3-kinase (PI3 kinase) inhibitor (LY-294002)³⁶ in 17 lung cell lines, and topotecan and a Src inhibitor (SU6656) in 13 ovarian cell lines was determined by quantifying the percent reduction in growth (versus DMSO controls) at 96 hrs using a standard MTT colorimetric assay.³⁶ Concentrations used ranged from 1-10 nM for docetaxel, 300 nM-10 μM (SU6656), and 300 nM-10 M for LY-294002. All experiments were repeated at least three times.

Statistical analysis methods. Analysis of expression data are as previously described.^{36, 60-62} Briefly, prior to statistical modeling, gene expression data is filtered to exclude probesets with signals present at background noise levels, and for probesets that do not vary significantly across samples. Each signature summarizes its constituent genes as a single expression profile, and is here derived as the top principal components of that set of genes. When predicting the chemosensitivity patterns or pathway activation of cancer cell lines or tumor samples, gene selection and identification is based on the training data, and then metagene values are computed using the principal components of the training data and additional cell line or tumor expression data. Bayesian fitting of binary probit regression models to the training data then permits an assessment of the relevance of the metagene signatures in within-sample classification,⁶⁰ and estimation and uncertainty assessments for the binary regression weights mapping metagenes to probabilities. To guard against over-fitting given the disproportionate number of variables to samples, we also performed leave-one-out cross validation analysis to test the stability and predictive capability of our model. Each sample was left out of the data set one at a time, the model was refitted (both the metagene factors and the partitions used) using the remaining samples, and the phenotype of the held out case was then predicted and the certainty of the classification was calculated. Given a training set of expression vectors (of values across metagenes) representing two biological states, a binary probit regression model, of predictive probabilities for each of the two states (resistant vs. sensitive) for each case is estimated using Bayesian methods. Predictions of the relative oncogenic pathway status and chemosensitivity of the validation cell lines or tumor samples are then evaluated using methods previously described^{36,60} producing estimated relative probabilities—and associated measures of uncertainty—of chemosensitivity/oncogenic pathway deregulation across the validation samples. In instances where a combined probability of sensitivity to a combination chemotherapeutic regimen was required based on the individual drug sensitivity patterns, we employed the theorem for combined probabilities as described by Feller: [Probability (Pr) of (A), (B), (C) . . . (N)]=ΣPr (A)+Pr (B)+Pr (C) . . . +Pr (N)−[Pr(A)×Pr(B)×Pr(C) . . . ×Pr (N)]. Hierarchical clustering of tumor predictions was performed using Gene Cluster 3.0.⁶³ Genes and tumors were clustered using average linkage with the uncentered correlation similarity metric. Standard linear regression analyses and their significance (log rank test) were generated for the drug response data and correlation between drug response and probability of chemosensitivity/pathway deregulation using GraphPad® software.

Reference Bibliography

1. Levin L, Simon R, Hryniuk W: Importance of multiagent chemotherapy regimens in ovarian carcinoma: dose intensity analysis. J. Natl. Canc. Inst. 85:1732-1742, 1993

2. McGuire W P, Hoskins W J, Brady M F, et al: Assessment of dose-intensive therapy in suboptimally debulked ovarian cancer: a Gynecologic Oncology Group study. J. Clin. Oncol. 13:1589-1599, 1995

3. Jodrell D I, Egorin M J, Canetta R M, et al: Relationships between carboplatin explosure and tumor response and toxicity in patients with ovarian cancer. J. Clin. Oncol. 10:520-528, 1992

4. McGuire W P, Hoskins W J, Brady M F, et al: Cyclophosphamide and cisplatin compared with paclitaxel and cisplatin in patients with stage III and stage IV ovarian cancer. N. Engl. J. Med. 334:1-6, 1996

5. McGuire W P, Brady M F, Ozols R F: The Gynecologic Oncology Group experience in ovarian cancer. Ann. Oncol. 10:29-34, 1999

6. Piccart M J, Bertelsen K, Stuart G, et al: Long-term follow-up confirms a survival advantage of the paclitaxel-cisplatin regimen over the cyclophosphamide-cisplatin combination in advanced ovarian cancer. Int. J. Gynecol. Cancer 13:144-148, 2003

7. Wenham R M, Lancaster J M, Berchuck A: Molecular aspects of ovarian cancer. Best Pract. Res. Clin. Obstet. Gynaecol. 16:483-497, 2002

8. Berchuck A, Kohler M F, Marks J R, et al: The p53 tumor suppressor gene frequently is altered in gynecologic cancers. Am. J. Obstet. Gynecol. 170:246-252, 1994

9. Kohler M F, Marks J R, Wiseman R W, et al: Spectrum of mutation and frequency of allelic deletion of the p53 gene in ovarian cancer. J. Natl. Canc. Inst. 85:1513-1519, 1993

10. Havrilesky L, Alvarez A A, Whitaker R S, et al: Loss of expression of the p16 tumor suppressor gene is more frequent in advanced ovarian cancers lacking p53 mutations. Gynecol. Oncol. 83:491-500, 2001

11. Reles A, Wen W H, Schmider A, et al: Correlation of p53 mutations with resistance to platinum-based chemotherapy and shortened survival in ovarian cancer. Clinical Cancer Research 7:2984-2997, 2001

12. Schmider A, Gee C, Friedmann W, et al: p21 (WAF1/CIP1) protein expression is associated with prolonged survival but not with p53 expression in epithelial ovarian carcinoma. Gynecol. Oncol. 77:237-242, 2000

13. Wong K K, Cheng R S, Mok S C: Identification of differentially expressed genes from ovarian cancer cells by MICROMAX cDNA microarray system. Biotechniques 30:670-675, 2001

14. Welsh J B, Zarrinkar P P, Sapinoso L M, et al: Analysis of gene expression profiles in normal and neoplastic ovarian tissue samples identifies candidate molecular markers of epithelial ovarian cancer. Proc. Natl. Acad. Sci. USA 98:1176-1181, 2001

15. Shridhar V, Lee J-S, Pandita A, et al: Genetic analysis of early-versus late-state ovarian tumors. Cancer Res. 61:5895-5904, 2001

16. Schummer M, Ng W W, Bumgarner R E, et al: Comparative hybridization of an array of 21,500 ovarian cDNAs for the discovery of genes overexpressed in ovarian carcinomas. Gene 238:375-385, 1999

17. Ono K, Tanaka T, Tsunoda T, et al: Identification by cDNA microarray of genes involved in ovarian carcinogenesis. Cancer Res. 60:5007-5011, 2000

18. Sawiris G P, Sherman-Baust C A, Becker K G, et al: Development of a highly specialized cDNA array for the study and diagnosis of epithelial ovarian cancer. Cancer Res. 62:2923-2928, 2002

19. Jazaeri A A, Yee C J, Sotiriou C, et al: Gene expression profiles of BRCA1-linked, BRCA2-linked, and sporadic ovarian cancers. J. Natl. Canc. Inst. 94:990-1000, 2002

20. Schaner M E, Ross D T, Ciaravino G, et al: Gene expression patterns in ovarian carcinomas. Mol. Biol. Cell 14:4376-4386, 2003

21. Lancaster J M, Dressman H, Whitaker R S, et al: Gene expression patterns that characterize advanced stage serous ovarian cancers. J. Surgical Gynecol. Invest. 11:51-59, 2004

22. Berchuck A, Iversen E S, Lancaster J M, et al: Patterns of gene expression that characterize long term survival in advanced serous ovarian cancers. Clin. Can. Res. 11:3686-3696, 2005

23. Berchuck A, Iversen E, Lancaster J M, et al: Prediction of optimal versus suboptimal cytoreduction of advanced stage serous ovarian cancer using microarrays. Am. J. Obstet. Gynecol. 190:910-925, 2004

24. Jazaeri A A, Awtrey Cs, Chandramouli G V, et al: Gene expression profiles associated with response to chemotherapy in epithelial ovarian cancers. Clin. Cancer Res. 11:6300-6310, 2005

25. Helleman J, Jansen M P, Span P N, et al: Molecular profiling of platinum resistant ovarian cancer. Int. J. Cancer 118:1963-1971, 2005

26. Spentzos D, Levine D A, Kolia s, et al: Unique gene expression profile based on pathologic response in epithelial ovarian cancer. J. Clin. Oncol. 23:7911-7918, 2005

27. Spentzos D, Levine D A, Ramoni M F, et al: Gene expression signature with independent prognostic significance in epithelial ovarian cancer. J. Clin. Oncol. 22:4700-4710, 2004

28. Miller A B, Hoogstraten B, Staquet M, et al: Reporting results of cancer treatment. Cancer 47:207-214, 1981

29. Rustin G J, Nelstrop A E, Bentzen S M, et al: Use of tumor markers in monitoring the course of ovarian cancer. Ann. Oncol. 10:21-27, 1999

30. Rustin G J, Nelstrop A E, McClean P, et al: Defining response of ovarian carcinoma to initial chemotherapy according to serum CA 125. J. Clin. Oncol. 14:1545-1551, 1996

31. Irizarry R A, Hobbs B, Collin F, et al: Exploration, normalization, and summaries of high density oligonucleotide array probe level data. Biostatistics 4:249-263, 2003

32. Bolstad B M, Irizarry R A, Astrand M, et al: A comparison of normalizaton methods for high density oligonucleotide array data based on variance and bias. Bioinformatics 19:185-193, 2003

33. Lucus J, Carvalho C, Wang Q, et al: Sparse statistical modeling in gene expression genomics. Cambridge, Cambridge University Press, 2006

34. Rich J, Jones B, Hans C, et al: Gene expression profiling and genetic markers in glioblastoma survival. Cancer Res. 65:4051-4058, 2005

35. Hans C, Dobra A, West M: Shotgun stochastic search for regression with many candidate predictors. JASA in press., 2006

36. Bild A, Yao G, Chang J T, et al: Oncogenic pathway signatures in human cancers as a guide to targeted therapies. Nature 439:353-357, 2006.

37. Gyorrfy B, Surowiak P, Kiesslich O, Denkert C, Schafer R, Dietel M, Lage H: Gene expression profiling of 30 cancer cell lines predicts resistance towards 11 anticancer drugs at clinically achieved concentrations. Int. J. Cancer 118(7):1699-712, 2006

38. Minna, J D, Gazdar, A F, Sprang, S R & Herz, J: Cancer. A bull's eye for targeted lung cancer therapy. Science 304: 1458-1461, 2004

39. Jemal et al., CA Cancer J. Clin., 53, 5-26, 2003

40. Cancer Facts and Figures: American Cancer Society, Atlanta, p. 11, 2002

41. Travis et al., Lung Cancer Principles and Practice, Lippincott-Raven, New York, pps. 361-395, 1996

42. Gazdar et al., Anticancer Res. 14:261-267,

43. Niklinska et al., Folia Histochem. Cytobiol. 39:147-148, 2001

44. Parker et al, CA Cancer J. Clin. 47:5-27, 1997

45. Chu et al, J. Nat. Cancer Inst. 88:1571-1579, 1996

46. Baker, V V: Salvage therapy for recurrent epithelial ovarian cancer. Hematol. Oncol. Clin. N. Am. 17: 977-988, 2003

47. Hansen, H H, Eisenhauer, E A, Hasen M, Neijt J P, Piccart M J, Sessa C, Thigpen J T: New cytostatis drugs in ovarian cancer. Ann. Oncol. 4:S63-S70, 1993.

48. Herrin, V E, Thigpen J T: Chemotherapy for ovarian cancer: current concepts. Semin. Surg. Oncol. 17:181-188, 1999

49. Staunton, J. E. et al. Chemosensitivity prediction by transcriptional profiling. Proc Natl Acad Sci USA 98:10787-19792, 2001

50. Chang, J. C. et al. Gene expression profiling for the prediction of therapeutic response to docetaxel in patients with breast cancer. Lancet 362:362-369, 2003

51. Emi, M., Kim, R., Tanabe, K., Uchida, Y. & toge, T. Targeted therapy against Bcl-2-related proteins in breast cancer cells. Breast Cancer Res 7: R940-R952, 2005

52. Takahashi, T. et al. Cyclin A-associated kinase activity is needed for paclitaxel sensitivity. Mol. Cancer Ther 4:1039-1046, 2005

53. Modi, S. et al. Phosphorylated/activated HER2 as a marker of clinical resistance to single agent taxane chemotherapy for metastatic breast cancer. Cancer Invest 23: 483-487, 2005

54. Langer, R. et al. Association of pretherapeutic expression of chemotherapy-related genes with response to neoadjuvant chemotherapy in Barrett carcinoma. Clin Cancer Res. 11: 7462-7469, 2005

55. Rouzier, R. et al. Breast cancer molecular subtypes respond differently to preoperative chemotherapy. Clin Cancer Res. 11: 5678-5685, 2005

56. Rouzier, R. et al. Microbubule-associated protein tau: a marker of paclitaxel sensitivity on breast cancer. Proc Natl Acad Sci USA 102: 8315-8320, 2005

57. DeVita, V. T., Hellman, S. & Rosenberg, S. A. Cancer: Principles and Practice of Oncology, Lippincott-Raven, Philadelphia, 2005

58. Herbst, R. S. et al. Clinical Cancer Advances 2005; Major research advances in cancer treatment, prevention, and screening—a report from the American Society of Clinical Oncology. J. Clin. Oncol. 24: 190-205, 2006

59. Broxterman, H. J. & Georgopapadakou, N. H. Anticancer therapeutics: Addictive targets, multi-targeted drugs, new drug combinations. Drug Resist Update 8:183-197, 2005

60. Pittman, J., Huang, E., Wang, Q., Nevins, J. R. & West, M. Bayesian analysis of binary prediction tree models for retrospectively sampled outcomes. Biostatistics 5: 587-601, 2004

61. West, M. et al. Predicting the clinical status of human breast cancer by using gene expression profiles. Proc Natl Acad Sci USA 98:11462-11467, 2001

62. Ihaka, R. & Gentleman, R. A language for data analysis and graphics. J. Comput. Graph. Stat. 5: 299-314, 1996

63. Eisen, M. B., Spellman, P. T., Brown, P. O. & Botstein, D. Cluster analysis and display of genome-wide expression patterns. Proc Natl Acad Sci USA 95:14863-14868, 1998

TABLE 1
Clinico-pathologic characteristics of ovarian cancer samples analyzed

| | Clinical Complete | Clinical Incomplete |
|---|---|---|
| | Responders | Responders |
| | (N = 85) | (N = 34) |
| Mean age (Yrs) | 63 | 65 |
| Stage (n) | | |
| III | 72 | 27 |
| IV | 13 | 7 |
| Grade (n) | | |
| I | 2 | 1 |
| II | 42 | 15 |
| III | 41 | 18 |
| Surgical Debulking (n) | | |
| Optimally (<1 cm) | 51 | 12 |
| Suboptimal (>1 cm) | 34 | 22 |
| Chemotherapy (n) | | |
| Platinum/Cytoxan | 23 | 11 |
| Platinum/Taxol | 60 | 22 |
| Single Agent Platinum | 2 | 1 |
| Mean Serum CA125 (u/ml) | | |
| Pre-platinum | 2601 | 4635 |
| Post-platinum | 16 | 529 |
| Mean Survival (Months) | 45 | 31 |

TABLE 2
Highest weighted genes in the platinum prediction response models using 83-
sample training set and validated in 36-sample validation set

| Gene Title | Gene Symbol | Representative Public ID |
|---|---|---|
| sialidase 1 (lysosomal sialidase) | NEU1 | U84246 |
| translocated promoter region (to activated | TPR | NM_003292 |
| MET oncogene) | | |
| periplakin | PPL | NM_002705 |
| H3 histone, family 3B (H3.3B) | H3F3B | BC001124 |
| zinc finger protein 264 | ZNF264 | NM_003417 |
| proteasome (prosome, macropain) 26S subunit, | PSMD4 | AB033605 |
| non-ATPase, 4 | | |
| heterogeneous nuclear ribonucleoprotein U | HNRPU | BC003621 |
| peptidylglycine alpha-amidating | PAM | NM_000919 |
| monooxygenase | | |
| glyceronephosphate O-acyltransferase | GNPAT | NM_014236 |
| splicing factor 3a, subunit 3, 60 kDa | SF3A3 | NM_006802 |
| glycine cleavage system protein H | GCSH | AW237404 |
| (aminomethyl carrier) | | |
| reticulocalbin 1, EF-hand calcium binding | RCN1 | NM_002901 |
| domain | | |
| hypothetical protein FLJ10404 | FLJ10404 | NM_019057 |
| trophinin associated protein (tastin) | TROAP | NM_005480 |
| tissue inhibitor of metalloproteinase 2 | TIMP2 | NM_003255 |
| ribosomal protein S20 | RPS20 | BF184532 |
| PTK7 protein tyrosine kinase 7 | PTK7 | NM_002821 |
| suppressor of cytokine signaling 5 | SOCS5 | AW664421 |
| NADH dehydrogenase (ubiquinone) | NDUFV1 | AF092131 |
| flavoprotein 1, 51 kDa | | |
| protein phosphatase 4, regulatory subunit 1 | PPP4R1 | NM_005134 |
| cysteine-rich, angiogenic inducer, 61 | CYR61 | NM_001554 |
| MCM4 minichromosome maintenance | MCM4 | AA604621 |
| deficient 4 | | |
| thyroid hormone receptor associated protein 1 | THRAP1 | AB011165 |
| calcyclin binding protein /// calcyclin binding | CACYBP | BC005975 |
| protein | | |
| hydroxysteroid (17-beta) dehydrogenase 12 | HSD17B12 | NM_016142 |
| DnaJ (Hsp40) homolog, subfamily C, member 9 | DNAJC9 | BE551340 |
| translocated promoter region (to activated | TPR | BF110993 |
| MET oncogene) | | |
| PERP, TP53 apoptosis effector | PERP | NM_022121 |
| importin 13 | IPO13 | NM_014652 |
| pleckstrin homology domain interacting | PHIP | BF224151 |
| protein | | |
| cyclin B2 | CCNB2 | NM_004701 |
| CDC5 cell division cycle 5-like (S. pombe) | CDC5L | NM_001253 |
| zinc finger protein 592 | ZNF592 | NM_014630 |
| Kazrin | KIAA1026 | AB028949 |
| Nuclear receptor coactivator 2 | NCOA2 | AI040324 |
| DKFZP564G2022 protein | DKFZP564G2022 | BG493972 |
| GK001 protein | GK001 | NM_020198 |
| IQ motif containing GTPase activating protein 1 | IQGAP1 | AI679073 |
| lysosomal associated protein transmembrane 4 | LAPTM4B | NM_018407 |
| beta | | |
| protein-kinase, interferon-inducible double | | |
| stranded RNAdependent inhibitor, repressor of | | |
| (P58 repressor) | | |
| ash2 (absent, small, or homeotic)-like | ASH2L | AB020982 |
| (Drosophila) | | |
| kallikrein 5 | KLK5 | AF243527 |
| low density lipoprotein-related protein 1 (alpha- | | |
| 2-macroglobulin receptor) | | |
| membrane-associated ring finger (C3HC4) 5 | C3HC4 | NM_017824 |
| ring-box 1 | RBX1 | NM_014248 |
| SET domain, bifurcated 1 | SETDB1 | NM_012432 |
| epiplakin 1 /// epiplakin 1 | EPPK1 | NM_031308 |
| HIV-1 Tat interacting protein, 60 kDa | HTATIP | BC000166 |
| CGI-128 protein | CGI-128 | NM_016062 |
| reticulon 3 | RTN3 | NM_006054 |
| CGI-62 protein | CGI-62 | NM_016010 |
| 7-dehydrocholesterol reductase | DHCR7 | AW150953 |
| chromosome 9 open reading frame 10 | C9orf10 | BE963765 |
| replication factor C (activator 1) 1 | RFC1 | NM_002913 |
| nuclear transcription factor Y, beta | NFYB | AI804118 |
| chromosome 8 open reading frame 33 | C8orf33 | NM_023080 |
| tumor rejection antigen (gp96) 1 | TRA1 | NM_003299 |
| transportin 1 | TNPO1 | NM_002270 |
| protein phosphatase 3 (formerly 2B), catalytic | PPP3CB | NM_021132 |
| subunit | | |
| high-mobility group 20B | HMG20B | BC002552 |
| Lamin A/C | LMNA | AA063189 |
| phosphoglycerate kinase 1 | PGK1 | NM_000291 |
| RNA (guanine-7-) methyltransferase | RNMT | NM_003799 |
| HSPC038 protein | LOC51123 | NM_016096 |
| myosin VI | MYO6 | AA877789 |
| lipase A, lysosomal acid, cholesterol esterase | LIPA | NM_000235 |
| DiGeorge syndrome critical region gene 6 /// | | |
| DiGeorge syndrome critical region gene 6-like | | |
| protein kinase C, zeta | PRKCZ | NM_002744 |
| tankyrase, TRF1-interacting ankyrin-related | | |
| ADP-ribose polymerase 2 | | |
| Nedd4 binding protein 1 | N4BP1 | BF436315 |
| tetraspanin 6 | TSPAN6 | AF053453 |
| mitochondrial ribosomal protein L9 /// | | |
| mitochondrial ribosomal protein L9 | | |
| chromosome 20 open reading frame 47 | C20orf47 | AF091085 |
| macrophage stimulating 1 (hepatocyte growth | MST1 | NM_020998 |
| factor-like) | | |
| Mlx interactor | MONDOA | NM_014938 |
| RAB31, member RAS oncogene family | RAB31 | NM_006868 |
| prosaposin (variant Gaucher disease and | | |
| variant metachromatic leukodystrophy) | | |
| solute carrier family 25 (mitochondrial carrier; | | |
| oxoglutarate carrier) | | |
| small nuclear ribonucleoprotein polypeptide A | SNRPA | NM_004596 |
| KIAA0247 | KIAA0247 | NM_014734 |
| cyclin M3 | CNNM3 | NM_017623 |
| zinc finger protein 443 | ZNF443 | NM_005815 |
| matrix-remodelling associated 5 | MXRA5 | AF245505 |
| RAE1 RNA export 1 homolog (S. pombe) | RAE1 | NM_003610 |
| ATP synthase, H+ transporting, mitochondrial | | |
| F0 complex, subunit d | | |
| Coenzyme A synthase | COASY | NM_025233 |
| mutS homolog 6 (E. coli) | MSH6 | NM_000179 |
| ubiquitin specific protease 25 | USP25 | NM_013396 |
| quiescin Q6 | QSCN6 | NM_002826 |
| adenylate kinase 2 | AK2 | W02312 |
| GNAS complex locus | GNAS | AI591100 |
| nucleolar protein family A, member 3 (H/ACA | | |
| small nucleolar RNPs) | | |
| phosphatidylinositol-4-phosphate 5-kinase, | PIP5K1C | AB011161 |
| type I, gamma | | |
| microtubule-associated protein 4 | MAP4 | W28892 |
| torsin family 3, member A | TOR3A | NM_022371 |
| ankyrin repeat domain 10 | ANKRD10 | NM_017664 |
| muscleblind-like (Drosophila) | MBNL1 | NM_021038 |
| shank-interacting protein-like 1 /// shank- | | |
| interacting protein-like 1 | | |
| natriuretic peptide receptor A/guanylate | | |
| cyclase A (atrionatriuretic peptide receptor A) | | |
| geranylgeranyl diphosphate synthase 1 | GGPS1 | NM_004837 |

TABLE 3

| Number of | | |
|---|---|---|
| Gene | | In |
| Ontology | | (Bayes |
| group | Annotation Name | factor) |
| 1 | GO:0001558 [4]: regulation of cell growth | 4.177 |
| 2 | GO:0040008 [4]: regulation of growth | 3.802 |
| 3 | GO:0016049 [4]: cell growth | 3.005 |
| 4 | GO:0008361 [5]: regulation of cell size | 3.005 |
| 5 | GO:0040007 [3]: growth | 2.044 |
| 6 | GO:0050793 [3]: regulation of development | 2.021 |
| 7 | GO:0016043 [4]: cell organization and | 1.955 |
| | biogenesis | |
| 8 | GO:0051169 [6]: nuclear transport | 1.896 |
| 9 | GO:0000902 [4]: cellular morphogenesis | 1.833 |
| 10 | GO:0006913 [6]: nucleocytoplasmic transport | 1.646 |
| 11 | GO:0000059 [8]: protein-nucleus import, | 1.175 |
| | docking | |
| 12 | GO:0007004 [9]: telomerase-dependent | 1.066 |
| | telomere maintenance | |
| 13 | GO:0000723 [8]: telomere maintenance | 0.964 |
| 14 | GO:0051170 [7]: nuclear import | 0.963 |
| 15 | GO:0006606 [7]: protein-nucleus import | 0.963 |
| 16 | GO:0045581 [7]: negative regulation of T-cell | 0.862 |
| | differentiation | |
| 17 | GO:0045623 [8]: negative regulation of T-helper | 0.862 |
| | cell differentiation | |
| 18 | GO:0045629 [9]: negative, regulation of T-helper | 0.862 |
| | 2 cell differentiation | |
| 19 | GO:0001519 [6]: peptide amidation | 0.862 |
| 20 | GO:0001522 [7]: pseudouridine synthesis | 0.862 |

TABLE 4
Topotecan Predictor Set of Gene Expression Profiles

| | | | | Representative |
|---|---|---|---|---|
| Probe Set ID | Gene Title | Gene Sym | UniGene | Public ID |
| 200050_at | zinc finger protein 146 /// zinc finger | ZNF146 | 301819 | NM_007145 |
| | protein 146 | | | |
| 200065_s_at | ADP-ribosylation factor 1 /// ADP- | ARF1 | 286221 | AF052179 |
| | ribosylation factor 1 | | | |
| 200077_s_at | ornithine decarboxylase antizyme 1 /// | OAZ1 | 446427 | D87914 |
| | ornithine decarboxylase antizyn | | | |
| 200710_at | acyl-Coenzyme A dehydrogenase, very | ACADVL | 437178 | NM_000018 |
| | long chain | | | |
| 200717_x_at | ribosomal protein L7 | RPL7 | 421257 | NM_000971 |
| 200819_s_at | ribosomal protein S15 | RPS15 | 406683 | NM_001018 |
| 200839_s_at | cathepsin B | CTSB | 520898 | NM_001908 |
| 200949_x_at | ribosomal protein S20 | RPS20 | 8102 | NM_001023 |
| 201193_at | isocitrate dehydrogenase 1 (NADP+), | IDH1 | 11223 | NM_005896 |
| | soluble | | | |
| 201219_at | C-terminal binding protein 2 /// | CTBP2 /// I | 501345 | AW269836 |
| | LOC440008 | | | |
| 201381_x_at | calcyclin binding protein | CACYBP | 508524 | AF057356 |
| 201434_at | tetratricopeptide repeat domain 1 | TTC1 | 519718 | NM_003314 |
| 201482_at | quiescin Q6 | QSCN6 | 518374 | NM_002826 |
| 201568_at | low molecular mass ubiquinone-binding | QP-C | 146602 | NM_014402 |
| | protein (9.5 kD) | | | |
| 201592_at | eukaryotic translation initiation factor 3, | EIF3S3 | 492599 | NM_003756 |
| | subunit 3 gamma, 40 kDa | | | |
| 201758_at | tumor susceptibility gene 101 | TSG101 | 523512 | NM_006292 |
| 201795_at | lamin B receptor | LBR | 435166 | NM_002296 |
| 201838_s_at | suppressor of Ty 7 (S. cerevisiae)-like | SUPT7L | 6232 | NM_014860 |
| 201848_s_at | BCL2/adenovirus E1B 19 kDa interacting | BNIP3 | 144873 | U15174 |
| | protein 3 | | | |
| 201867_s_at | transducin (beta)-like 1X-linked | TBL1X | 495656 | AW968555 |
| 202000_at | NADH dehydrogenase (ubiquinone) 1 | NDUFA6 | 274416 | BC002772 |
| | alpha subcomplex, 6, 14 kDa | | | |
| 202042_at | histidyl-tRNA synthetase | HARS | 528050 | NM_002109 |
| 202087_s_at | cathepsin L | CTSL | 418123 | NM_001912 |
| 202090_s_at | ubiquinol-cytochrome c reductase, | UQCR | 8372 | NM_006830 |
| | 6.4 kDa subunit | | | |
| 202138_x_at | JTV1 gene | JTV1 | 301613 | NM_006303 |
| 202144_s_at | adenylosuccinate lyase | ADSL | 75527 | NM_000026 |
| 202223_at | integral membrane protein 1 | ITM1 | 504237 | NM_002219 |
| 202282_at | hydroxyacyl-Coenzyme A | HADH2 | 171280 | NM_004493 |
| | dehydrogenase, type II | | | |
| 202445_s_at | Notch homolog 2 (Drosophila) | NOTCH2 | 549056 | NM_024408 |
| 202472_at | mannose phosphate isomerase | MPI | 75694 | NM_002435 |
| 202618_s_at | methyl CpG binding protein 2 (Rett | MECP2 | 200716 | L37298 |
| | syndrome) | | | |
| 202619_s_at | procollagen-lysine, 2-oxoglutarate 5- | PLOD2 | 477866 | AI754404 |
| | dioxygenase 2 | | | |
| 202639_s_at | RAN binding protein 3 | RANBP3 | 531752 | AI689052 |
| 202745_at | Ubiquitin specific protease 8 | USP8 | 443731 | NM_005154 |
| 202780_at | 3-oxoacid CoA transferase 1 | OXCT1 | 278277 | NM_000436 |
| 202823_at | Transcription elongation factor B (SIII), | TCEB1 | 546305 | N89607 |
| | polypeptide 1 (15 kDa, elongin | | | |
| 202824_s_at | transcription elongation factor B (SIII), | TCEB1 | 546305 | NM_005648 |
| | polypeptide 1 (15 kDa, elongin | | | |
| 202846_s_at | phosphatidylinositol glycan, class C | PIGC | 188456 | NM_002642 |
| 202892_at | CDC23 (cell division cycle 23, yeast, | CDC23 | 153546 | NM_004661 |
| | homolog) | | | |
| 202944_at | N-acetylgalactosaminidase, alpha- | NAGA | 75372 | NM_000262 |
| 203013_at | suppressor of S. cerevisiae gcr2 | HSGT1 | 446373 | NM_007265 |
| 203039_s_at | NADH dehydrogenase (ubiquinone) Fe—S | NDUFS1 | 471207 | NM_005006 |
| | protein 1, 75 kDa (NADH-co | | | |
| 203164_at | solute carrier family 33 (acetyl-CoA | SLC33A1 | 478031 | BE464756 |
| | transporter), member 1 | | | |
| 203207_s_at | chondrocyte protein with a poly-proline | CHPPR | 521608 | BF214329 |
| | region | | | |
| 203223_at | rabaptin, RAB GTPase binding effector | RABEP1 | 551518 | NM_004703 |
| | protein 1 | | | |
| 203228_at | platelet-activating factor | PAFAH1B3 | 466831 | NM_002573 |
| | acetylhydrolase, isoform lb, gamma | | | |
| | subunit 2 | | | |
| 203269_at | neutral sphingomyelinase (N-SMase) | NSMAF3 | 372000 | NM_003580 |
| | activation associated factor | | | |
| 203282_at | glycan (1,4-alpha-), branching enzyme 1 | GBE1 | 436062 | NM_000158 |
| | (glycogen branching enzyme | | | |
| 203321_s_at | KIAA0863 protein | KIAA0863 | 131915 | AK022688 |
| 203521_s_at | zinc finger protein 318 | ZNF318 | 509718 | NM_014345 |
| 203538_at | calcium modulating ligand | CAMLG | 529846 | NM_001745 |
| 203591_s_at | colony stimulating factor 3 receptor | CSF3R | 524517 | NM_000760 |
| | (granulocyte) /// colony stimulating | | | |
| 203747_at | aquaporin 3 | AQP3 | 234642 | NM_004925 |
| 203912_s_at | deoxyribonuclease I-like 1 | DNASE1L1 | 77091 | NM_006730 |
| 203957_at | E2F transcription factor 6 | E2F6 | 135465 | NM_001952 |
| 204028_s_at | RAB GTPase activating protein 1 | RABGAP1 | 271341 | NM_012197 |
| 204091_at | phosphodiesterase 6D, cGMP-specific, | PDE6D | 516808 | NM_002601 |
| | rod, delta | | | |
| 204185_x_at | peptidylprolyl isomerase D (cyclophilin | PPID | 183958 | NM_005038 |
| | D) | | | |
| 204226_at | staufen, RNA binding protein, homolog | STAU2 | 350756 | NM_014393 |
| | 2 (Drosophila) | | | |
| 204366_s_at | general transcription factor IIIC, | GTF3C2 | 75782 | NM_001521 |
| | polypeptide 2, beta 110 kDa | | | |
| 204381_at | low density lipoprotein receptor-related | LRP3 | 515340 | NM_002333 |
| | protein 3 | | | |
| 204386_s_at | mitochondrial ribosomal protein 63 | MRP63 | 458367 | BF303597 |
| 204392_at | calcium/calmodulin-dependent protein | CAMK1 | 434875 | NM_003656 |
| | kinase I | | | |
| 204489s_at | CD44 antigen (homing function and | CD44 | 502328 | NM_000610 |
| | Indian blood group system) | | | |
| 204490s_at | CD44 antigen (homing function and | CD44 | 502328 | M24915 |
| | Indian blood group system) | | | |
| 204657_s_at | Src homology 2 domain containing | SHB | 521482 | NM_003028 |
| | adaptor protein B | | | |
| 204688_at | sarcoglycan, epsilon | SGCE | 371199 | NM_003919 |
| 204766_s_at | nudix (nucleoside diphosphate linked | NUDT1 | 534331 | NM_002452 |
| | moiety X)-type motif 1 | | | |
| 204925_at | cystinosis, nephropathic | CTNS | 187667 | NM_004937 |
| 204964_s_at | sarcospan (Kras oncogene-associated | SSPN | 183428 | NM_005086 |
| | gene) | | | |
| 204983_s_at | glypican 4 | GPC4 | 58367 | AF064826 |
| 204984_at | glypican 4 | GPC4 | 58367 | NM_001448 |
| 205068_s_at | Rho GTPase activating protein 26 | ARHGAP2 | 293593 | BE671084 |
| 205090_s_at | N-acetyiglucosamine-1-phosphodiester | NAGPA | 21334 | NM_016256 |
| | alpha-N-acetylglucosaminidas | | | |
| 205153_s_at | CD40 antigen (TNF receptor | CD40 | 472860 | NM_001250 |
| | superfamily member 5) | | | |
| 205164_at | glycine C-acetyltransferase (2-amino-3- | GCAT | 54609 | NM_014291 |
| | ketobutyrate coenzyme A ligas | | | |
| 205173_x_at | CD58 antigen, (lymphocyte function- | CD58 | 34341 | NM_001779 |
| | associated antigen 3) | | | |
| 205598_at | TRAF interacting protein | TRIP | 517972 | NM_005879 |
| 205729_at | oncostatin M receptor | OSMR | 120658 | NM_003999 |
| 205841_at | Janus kinase 2 (a protein tyrosine | JAK2 | 434374 | NM_004972 |
| | kinase) | | | |
| 205857_at | — | — | — | AI269290 |
| 206017_at | KIAA0319 | KIAA0319 | 26441 | NM_014809 |
| 206055_s_at | small nuclear ribonucleoprotein | SNRPA1 | 528763 | NM_003090 |
| | polypeptide A′ | | | |
| 206369_s_at | phosphoinositide-3-kinase, catalytic, | PIK3CG | 32942 | AF327656 |
| | gamma polypeptide | | | |
| 206417_at | cyclic nucleotide gated channel alpha 1 | CNGA1 | 1323 | NM_000087 |
| 206441_s_at | COMM domain containing 4 | COMMD4 | 351327 | NM_017828 |
| 206457_s_at | deiodinase, iodothyronine, type I | DIO1 | 251415 | NM_000792 |
| 206525_at | gamma-aminobutyric acid (GAGA) | GABRR1 | 437745 | NM_002042 |
| | receptor, rho 1 | | | |
| 206527_at | 4-aminobutyrate aminotransferase | ABAT | 336768 | NM_000663 |
| 206562_s_at | casein kinase 1, alpha 1 | CSNK1A1 | 442592 | NM_001892 |
| 206592_s_at | adaptor-related protein complex 3, delta | AP3D1 | 512815 | NM_003938 |
| | 1 subunit | | | |
| 206821_x_at | HIV-1 Rev binding protein-like | HRBL | 521083 | NM_006076 |
| 206857_s_at | FK506 binding protein 1B, 12.6 kDa | FKBP1B | 306834 | NM_004116 |
| 206860_s_at | hypothetical protein FLJ20323 | FLJ20323 | 520215 | NM_019005 |
| 206925_at | ST8 alpha-N-acetyl-neureminide alpha- | ST8SIA4 | 308628 | NM_005668 |
| | 2,8-sialyltransferase 4 | | | |
| 207156_at | histone 1, H2ag | HIST1H2A | 51011 | NM_021064 |
| 207168_s_at | H2A histone family, member Y | H2AFY | 420272 | NM_004893 |
| 207196_s_at | TNFAIP3 interacting protein 1 | TNIP1 | 355141 | NM_006058 |
| 207206_s_at | arachidonate 12-lipoxygenase | ALOX12 | 422967 | NM_000697 |
| 207348_s_at | ligase III, DNA, ATP-dependent | LIG3 | 100299 | NM_002311 |
| 207498_s_at | cytochrome P450, family 2, subfamily D, | CYP2D6 | 534311 | NM_000106 |
| | polypeptide 6 | | | |
| 207565_s_at | major histocompatibility complex, class | MR1 | 101840 | NM_001531 |
| | I-related | | | |
| 207802_at | cysteine-rich secretory protein 3 | CRISP3 | 404466 | NM_006061 |
| 208638_at | protein disulfide isomerase family A, | PDIA6 | 212102 | BE910010 |
| | member 6 | | | |
| 208644_at | poly (ADP-ribose) polymerase family, | PARP1 | 177766 | M32721 |
| | member 1 | | | |
| 208755_x_at | H3 histone, family 3A | H3F3A | 533624 | BF312331 |
| 208813_at | glutamic-oxaloacetic transaminase 1, | GOT1 | 500755 | BC000498 |
| | soluble (aspartate aminotransfe | | | |
| 208815_x_at | heat shock 70 kDa protein 4 | HSPA4 | 90093 | AB023420 |
| 208936_x_at | lectin, galactoside-binding, soluble, 8 | LGALS8 | 4082 | AF074000 |
| | (galectin 8) | | | |
| 208996_s_at | polymerase (RNA) II (DNA directed) | POLR2C | 79402 | BC000409 |
| | polypeptide C, 33 kDa | | | |
| 209036_s_at | malate dehydrogenase 2, NAD | MDH2 | 520967 | BC001917 |
| | (mitochondrial) | | | |
| 209104_s_at | nucleolar protein family A, member 2 | NOLA2 | 27222 | BC000009 |
| | (H/ACA small nucleolar RNPs) | | | |
| 209108_at | tetraspanin 6 | TSPAN6 | 43233 | AF053453 |
| 209224_s_at | NADH dehydrogenase (ubiquinone) 1 | NDUFA2 | 534333 | BC003674 |
| | alpha subcomplex, 2, 8 kDa | | | |
| 209232_s_at | dynactin 4 | MGC3248 | 435941 | BC004191 |
| 209289_at | Nuclear factor I/B | NFIB | 370359 | AI700518 |
| 209290_s_at | nuclear factor I/B | NFIB | 370359 | BC001283 |
| 209337_at | PC4 and SFRS1 interacting protein 1 | PSIP1 | 493516 | AF063020 |
| 209354_at | tumor necrosis factor receptor | TNFRSF14 | 512898 | BC002794 |
| | superfamily, member 14 (herpesvirus | | | |
| 209445_x_at | hypothetical protein FLJ10803 | FLJ10803 | 289007 | AI765280 |
| 209466_x_at | pleiotrophin (heparin binding growth | PTN | 371249 | M57399 |
| | factor 8, neurite growth-promoting | | | |
| 209482_at | processing of precursor 7, ribonuclease | POP7 | 416994 | BC001430 |
| | P subunit (S. cerevisiae) | | | |
| 209490_s_at | palmitoyl-protein thioesterase 2 | PPT2 | 332138 | AF020543 |
| 209540_at | insulin-like growth factor 1 | IGF1 | 160562 | AU144912 |
| | (somatomedin C) | | | |
| 209542_x_at | insulin-like growth factor 1 | IGF1 | 160562 | M29644 |
| | (somatomedin C) | | | |
| 209591_s_at | bone morphogenetlc protein 7 | BMP7 | 473163 | M60316 |
| | (osteogenic protein 1) | | | |
| 209593_s_at | torsin family 1, member B (torsin B) | TOR1B | 252682 | AF317129 |
| 209731_at | nth endonuclease III-like 1 (E. coli) | NTHL1 | 66196 | U79718 |
| 209813_x_at | T cell receptor gamma constant 2 /// T | TRGC2 /// | 534032 | M16768 |
| | cell receptor gamma constant | | | |
| 209822_s_at | very low density lipoprotein receptor | VLDLR | 370422 | L22431 |
| 209835_x_at | CD44 antigen (homing function and | CD44 | 502328 | BC004372 |
| | Indian blood group system) | | | |
| 209940_at | poly (ADP-ribose) polymerase family, | PARP3 | 271742 | AF083068 |
| | member 3 | | | |
| 210253_at | HIV-1 Tat interactive protein 2, 30 kDa | HTATIP2 | 90753 | AF092095 |
| 210347_s_at | B-cell CLL/lymphoma 11A (zinc finger | BCL11A | 370549 | AF080216 |
| | protein) | | | |
| 210538_s_at | baculoviral IAP repeat-containing 3 | BIRC3 | 127799 | U37546 |
| 210554_s_at | C-terminal binding protein 2 | CTBP2 | 501345 | BC002486 |
| 210586_x_at | Rhesus blood group, D antigen | RHD | 269364 | AF312679 |
| 210691_s_at | calcyclin binding protein | CACYBP | 508524 | AF275803 |
| 210916_s_at | CD44 antigen (homing function and | CD44 | 502328 | AF098641 |
| | Indian blood group system) | | | |
| 211259_s_at | bone morphogenetic protein 7 | BMP7 | 473163 | BC004248 |
| | (osteogenic protein 1) | | | |
| 211303_x_at | prostate-specific membrane antigen-like | PSMAL | — | AF261715 |
| 211355_x_at | leptin receptor | LFPR | 23581 | U52914 |
| 211363_s_at | methylthioadenosine phosphorylase | MTAP | 193268 | AF109294 |
| 211596_s_at | leucine-rich repeats and | LRIG1 | 518055 | AB050468 |
| | immunoglobulin-like domains 1 /// | | | |
| | leucine-ric | | | |
| 211737_x_at | pleiotrophin (heparin binding growth | PTN | 371249 | BC005916 |
| | factor 8, neurite growth-promoting | | | |
| 211744_s_at | CD58 antigen, (lymphocyte function- | CD58 | 34341 | BC005930 |
| | associated antigen 3) /// CD58 ar | | | |
| 211828_s_at | TRAF2 and NCK interacting kinase | TNIK | 34024 | AF172268 |
| 211925_s_at | phospholipase C, beta 1 | PLCB1 | 310537 | AY004175 |
| | (phosphoirnositide-specific) | | | |
| 211940_x_at | H3 histone, family 3A /// H3 histone, | H3F3A /// L | 533624 | BE869922 |
| | family 3A pseudogene | | | |
| 212014_x_at | CD44 antigen (homing function and | CD44 | 502328 | AI493245 |
| | Indian blood group system) | | | |
| 212038_s_at | voltage-dependent anion channel 1 | VDAC1 | 202085 | AL515918 |
| 212063_at | CD44 antigen (homing function and | CD44 | 502328 | BE903880 |
| | Indian blood group system) | | | |
| 212084_at | testis expressed sequence 261 | TEX261 | 516087 | AV759552 |
| 212132_at | family with sequence similarity 61, | FAM61A | 407368 | AL117499 |
| | member A | | | |
| 212137_at | La ribonucleoprotein domain family, | LARP1 | 292078 | AV746402 |
| | member 1 | | | |
| 212348_s_at | amine oxidase (flavin containing) | AOF2 | 549117 | AB011173 |
| | domain 2 | | | |
| 212369_at | zinc finger protein 384 | ZNF384 | 103315 | AI264312 |
| 212449_s_at | lysophospholipase I | LYPLA1 | 435850 | BG288007 |
| 212867_at | Nuclear receptor coactivator 2 /// | NCOA2 | 446678 | AI040324 |
| | Nuclear receptor coactivator 2 | | | |
| 212880_at | WD repeat domain 7 | WDR7 | 465213 | AB011113 |
| 212957_s_at | hypothetical protein LOC92249 | LOC92249 | 31532 | AU154785 |
| 213029_at | Nuclear factor I/B | NFIB | 370359 | BG478428 |
| 213032_at | Nuclear factor I/B | NFIB | 370359 | AI186739 |
| 213033_s_at | Nuclear factor I/B | NFIB | 370359 | AI186739 |
| 213228_at | phosphodiesterase 8B | PDE8B | 78106 | AK023913 |
| 213346_at | hypothetical protein BC015148 | LOC93081 | 398111 | BE748563 |
| 213508_at | chromosome 14 open reading frame | C14orf147 | 269909 | AA142942 |
| | 147 | | | |
| 213538_at | SON DNA binding protein | SON | 517262 | AI936458 |
| 213828_x_at | H3 histone, family 3A /// H3 histone, | H3F3A /// L | 533624 | AA477655 |
| | family 3A pseudogene | | | |
| 214075_at | neuron derived neurotrophic factor | NENF | 461787 | AI984136 |
| 214117_s_at | biotinidase | BTD | 517830 | AI767414 |
| 214279_s_at | NDRG family member 2 | NDRG2 | 525205 | W74452 |
| 214319_at | Hypothetical protein CG003 | 13CDNA73 | 507669 | W58342 |
| 214542_x_at | histone 1, H2ai | HIST1H2A | 352225 | NM_003509 |
| 214736_s_at | adducin 1 (alpha) | ADD1 | 183706 | BE898639 |
| 214833_at | transmembrane protein 63A | TMEM63A | 119387 | AB007958 |
| 214943_s_at | RNA binding motif protein 34 | RBM34 | 535224 | D38491 |
| 214964_at | Trinucleotide repeat containing 18 | TNRC18 | 410404 | AA554430 |
| 215001_s_at | glutamate-ammonia ligase (glutamine | GLUL | 518525 | AL161952 |
| | synthase) | | | |
| 215023_s_at | peroxisome biogenesis factor 1 | PEX1 | 164682 | AC000064 |
| 215107_s_at | hypothetical protein FLJ20619 | FLJ20619 | 16230 | AI923972 |
| 215133_s_at | similar to KIAA0752 protein | LOC38934 | 368516 | AL117630 |
| 215214_at | Immunoglobulin lambda variable 3-21 | IGLC2 | 449585 | H53689 |
| 215425_at | BTG family, member 3 | BTG3 | 473420 | AL049332 |
| 215458_s_at | SMAD specific E3 ubiquitin protein | SMURF1 | 189329 | AF199364 |
| | ligase 1 | | | |
| 215587_x_at | phospholipase C, beta 1 | PLCB1 | 310537 | AA393484 |
| | (phosphoinositide-specific) | | | |
| 215734_at | chromosome 19 open reading frame 36 | C19orf36 | 424049 | AW182303 |
| 215737_x_at | upstream transcription factor 2, c-fos | USF2 | 454534 | X90824 |
| | interacting | | | |
| 215819_s_at | Rhesus blood group, CcEe antigens /// | RHCE /// R | 269364 | N53959 |
| | Rhesus blood group, D antigen | | | |
| 216221_s_at | pumilio homolog 2 (Drosophila) | PUM2 | 467824 | D87078 |
| 216294_s_at | KIAA1109 | KIAA1109 | 408142 | AL137254 |
| 216308_x_at | glyoxylate reductase/hydroxypyruvate | GRHPR | 155742 | AK026752 |
| | reductase | | | |
| 216583_x_at | — | — | — | AC004079 |
| 216985_s_at | syntaxin 3A | STX3A | 530733 | AJ002077 |
| 217388_s_at | kynureninase (L-kynurenine hydrolase) | KYNU | 470126 | D55639 |
| 217441_at | ubiguitin specific protease 33 | USP33 | 480597 | AK023664 |
| 217489_s_at | interleukin 6 receptor | IL6R | 135087 | S72848 |
| 217523_at | CD44 antigen (homing function and | CD44 | 502328 | AV700298 |
| | Indian blood group system) | | | |
| 217620_s_at | phosphoinositide-3-kinase, catalytic, | PIK3CB | 239818 | AA805318 |
| | beta polypeptide | | | |
| 217829_s_at | ubiguitin specific protease 39 | USP39 | 469173 | NM_006590 |
| 217852_s_at | ADP-ribosylation factor-like 10C | ARL10C | 250009 | NM_018184 |
| 217939_s_at | aftiphilin protein | AFTIPHILII | 468760 | NM_017657 |
| 217981_s_at | fracture callus 1 homolog (rat) | FXC1 | 54943 | NM_012192 |
| 218027_at | mitochondrial ribosomal protein L15 | MRPL15 | 18349 | NM_014175 |
| 218046_s_at | mitochondrial ribosomal protein S16 | MRPS16 | 180312 | NM_016065 |
| 218069_at | XTP3-transactivated protein A | XTP3TPA | 237971 | NM_024096 |
| 218071_s_at | makorin, ring finger protein, 2 | MKRN2 | 279474 | NM_014160 |
| 218107_at | WD repeat domain 26 | WDR26 | 497873 | NM_025160 |
| 218128_at | nuclear transcription factor Y, beta | NFYB | 84928 | AU151875 |
| 218134_s_at | RNA binding motif protein 22 | RBM22 | 202023 | NM_018047 |
| 218″158_s_at | adaptor protein containing pH domain, | APPL | 476415 | NM_012096 |
| | PTB domain and leucine zippe | | | |
| 218190_s_at | ubiquinol-cytochrome c reductase | UCRC | 284292 | NM_013387 |
| | complex (7.2 kD) | | | |
| 218219_s_at | LanC lantibiotic synthetase component | LANCL2 | 224282 | NM_018697 |
| | C-like 2 (bacterial) | | | |
| 218234_at | inhibitor of growth family, member 4 | ING4 | 524210 | NM_016162 |
| 218270_at | mitochondrial ribosomal protein L24 | MRPL24 | 418233 | NM_024540 |
| 218320_s_at | NADH dehydrogenase (ubiquinone) 1 | NDUFB11 | 521969 | NM_019056 |
| | beta subcomplex, 11, 17.3 kDa | | | |
| 218339_at | mitochondrial ribosomal protein L22 | MRPL22 | 483924 | NM_014180 |
| 218370_s_at | S100P binding protein Riken | S100PBPF | 440880 | NM_022753 |
| 218498_s_at | ERO1-like (S. cerevisiae) | ERO1L | 525339 | NM_014584 |
| 218618_s_at | fibronectin type III domain containing 3B | FNDC3B | 159430 | NM_022763 |
| 218642_s_at | coiled-coil-helix-coiled-coil-helix domain | CHCHD7 | 436913 | NM_024300 |
| | containing 7 | | | |
| 218688_at | DKFZP586B1621 protein | DKFZP586 | 6278 | NM_015533 |
| 218728_s_at | cornichon homolog 4 (Drosophila) | CNIH4 | 445890 | NM_014184 |
| 218901_at | phospholipid scramblase 4 | PLSCR4 | 477869 | NM_020353 |
| 219032_x_at | opsin 3 (encephalopsin, panopsin) | OPN3 | 534399 | NM_014322 |
| 219161_s_at | chemokine-like factor | CKLF | 15159 | NM_016951 |
| 219220_x_at | mitochondrial ribosomal protein S22 | MRPS22 | 550524 | NM_020191 |
| 219231_at | nuclear receptor coactivator 6 | NCOA6IP | 335068 | NM_024831 |
| | interacting protein | | | |
| 219497_s_at | B-cell CLL/lymphoma 11A (zinc finger | BCL11A | 370549 | NM_022893 |
| | protein) | | | |
| 219498_s_at | B-cell CLL/lymphoma 11A (zinc finger | BCL11A | 370549 | NM_018014 |
| | protein) | | | |
| 219518_s_at | elongation factor RNA polymerase II-like 3 | ELLS | 171466 | NM_025165 |
| 219630_at | PDZK1 interacting protein 1 | PDZK1IP1 | 431099 | NM_005764 |
| 219762_s_at | ribosomal protein L36 | RPL36 | 408018 | NM_015414 |
| 219800_s_at | — | — | — | NM_024838 |
| 219809_at | WD repeat domain 55 | WDR55 | 286261 | NM_017706 |
| 219818_s_at | G patch domain containing 1 | GPATC1 | 466436 | NM_018025 |
| 219933_at | glutaredoxin 2 | GLRX2 | 458283 | NM_016066 |
| 219966_x_at | BTG3 associated nuclear protein | BANP | 461705 | NM_017869 |
| 220083_x_at | ubiquitin carboxyl-terminal hydrolase L5 | UCHL5 | 145469 | NM_016017 |
| 220085_at | helicase, lymphoid-specific | HELLS | 546260 | NM_018063 |
| 220144_s_at | ankyrin repeat domain 5 | ANKRD5 | 70903 | NM_022096 |
| 221045_s_at | period homolog 3 (Drosophila) | PER3 | 533339 | NM_016831 |
| 221204_s_at | cartilage acidic protein 1 | CRTAC1 | 500741 | NM_018058 |
| 221504_s_at | ATPase, H+ transporting, lysosomal | ATP6V1H | 491737 | AF112204 |
| | 50/57 kDa, V1 subunit H | | | |
| 221522_at | ankyrin repeat domain 27 (VPS9 | ANKRD27 | 59236 | AL136784 |
| | domain) | | | |
| 221523_s_at | Ras-related GTP binding D | RRAGD | 485938 | AL138717 |
| 221524_s_at | Ras-related GTP binding D | RRAGD | 485938 | AF272036 |
| 221586_s_at | E2F transcription factor 5, p130-binding | E2F5 | 445758 | U15642 |
| 221654_s_at | ubiquitin specific protease 3 | USP3 | 458499 | AF077040 |
| 221739_at | chromosome 19 open reading frame 10 | C19orf10 | 465645 | AL524093 |
| 221776_s_at | bromodomain containing 7 | BRD7 | 437894 | AI885109 |
| 221792_at | RAB6B, member RAS oncogene family | RAB6B | 552596 | AW118072 |
| 221826_at | similar to RIKEN cDNA 2610307121 | LOC90806 | 157078 | BE671941 |
| 221896_s_at | likely ortholog of mouse hypoxia | HIG1 | 7917 | BE739519 |
| | induced gene 1 | | | |
| 221928_at | acetyl-Coenzyme A carboxylase beta | ACACB | 234898 | AI057637 |
| 222099_s_at | family with sequence similarity 61, | FAM61A | 407368 | AW593859 |
| | member A | | | |
| 222206_s_at | nicalin homolog (zebrafish) | NCLN | 501420 | AA781143 |
| 222362_at | insulin receptor substrate 3-like | IRS3L | — | H07885 |
| 34858_at | potassium channel tetramerisation | KCTD2 | 514468 | D79998 |
| | domain containing 2 | | | |
| 43427_at | acetyl-Coenzyme A carbaxylase beta | ACACB | 234898 | AI970898 |
| 49452_at | acetyl-Coenzyme A carbaxylase beta | ACACB | 234898 | AI057637 |
| 1 | GO:0019752 [6]: carboxylic acid | 18 [show] | | |
| | metabolism | | | |
| 2 | GO:0006091 [5]: generation of | 22 [show] | | |
| | precursor metabolites and energy | | | |
| 3 | GO:0006082 [5]: organic acid | 18 [show] | | |
| | metabolism | | | |
| 4 | GO:0007186 [6]: G-protein coupled | 4 [show] | | |
| | receptor protein signaling pathwa . . . | | | |
| 5 | GO:0044249 [5]: cellular biosynthesis | 30 [show] | | |
| 6 | GO:0009058 [4]: biosynthesis | 31 [show] | | |
| 7 | GO:0006519 [5]: amino acid and | 12 [show] | | |
| | derivative metabolism | | | |
| 8 | GO:0006118 [6]: electron transport | 14 [show] | | |
| 9 | GO:0009987 [2]: cellular process | 168 [show] | | |
| 10 | GO:0051084 [8]: posttranslational | 2 [show] | | |
| | protein folding | | | |
| 7 | GO:0006519 [5]: amino acid and | 12 [show] | | |
| | derivative metabolism | | | |
| 8 | GO:0006118 [6]: electron transport | 14 [show] | | |
| 9 | GO:0009987 [2]: cellular process | 168 [show] | | |
| 10 | GO:0051084 [8]: posttranslational | 2 [show] | | |
| | protein folding | | | |
| 11 | GO:0051085 [9]: chaperone cofactor | 2 [show] | | |
| | dependent protein folding | | | |
| 12 | GO:0050874 [3]: organismal | 18 [show] | | |
| | physiological process | | | |
| 13 | GO:0009308 [5]: amine metabolism | 12 [show] | | |
| 14 | GO:0006412 [6]: protein biosynthesis | 17 [show] | | |
| 15 | GO:0006100 [8]: tricarboxylic acid cycle | 3 [show] | | |
| | intermediate metabolism | | | |
| 16 | GO:0007166 [5]: cell surface receptor | 13 [show] | | |
| | linked signal transduction | | | |

TABLE 5
Genes constituting the individual chemosensitivity predictors

| | | | |
|---|---|---|---|
| Probe Set | | | Chromosomal |
| ID | Gene Title | Gene Symbol | Location |

5-FU PREDICTOR - Metagene 1

| | | | |
|---|---|---|---|
| 1519_at | v-ets erythroblastosis virus E26 oncogene homolog 2 (avian) | ETS2 | 21q22.3\|21q22.2 |
| 1711_at | tumor protein p53 binding protein, 1 | TP53BP1 | 15q15-q21 |
| 1881_at | | | |
| 31321_at | | | |
| 31725_s_at | ATP-binding cassette, sub-family A (ABC1), member 2 | ABCA2 | 9q34 |
| 32307_s_at | collagen, type I, alpha 2 | COL1A2 | 7q22.1 |
| 32317_s_at | sulfotransferase family, cytosolic, 1A, phenol-preferring, | SULT1A2 | 16p12.1 |
| | member 2 | | |
| | sulfotransferase family, cytosolic, 1A, phenol-preferring, | SULT1A1 | 16p11.2 |
| | member 1 | | |
| | sulfotransferase family, cytosolic, 1A, phenol-preferring, | SULT1A3 | |
| | member 3 | | |
| | sulfotransferase family, cytosolic, 1A, phenol-preferring, | SULT1A4 | |
| | member 4 | | |
| 32609_at | histone 2, H2aa | HIST2H2AA | 1q21.2 |
| 32754_at | tropomyosin 3 | TPM3 | 1q21.2 |
| 33436_at | SRY (sex determining region Y)-box 9 (campomelic | SOX9 | 17q24.3-q25.1 |
| | dysplasia, autosomal sex-reversal) | | |
| 33443_at | serine incorporator 1 | SERINC1 | 6q22.31 |
| 33658_at | Methytrahydofolate reductase gene 2 | MTHFR | 1q44 |
| 34376_at | protein kinase (cAMP-dependent, catalytic) inhibitor gamma | PKIG | 20q12-q13.1 |
| 34453_at | Cytochrome P450, family 2, subfamily B, polypeptide 7 | CYP2A7P1 | 19q13.2 |
| | pseudogene 1 | | |
| 34544_at | zinc finger protein 267 | ZNF267 | 16p11.2 |
| 34842_at | small nuclear ribonucleoprotein polypeptide N | SNRPN | 15q11.2 |
| | SNRPN upstream reading frame | SNURF | 15q12 |
| 34904_at | glutamate receptor, ionotropic, kainate 5 | GRIK5 | 19q13.2 |
| 34953_i_at | phosphodiesterase 5A, cGMP-specific | PDE5A | 4q25-q27 |
| 35055_at | basic transcription factor 3 | BTF3 | 5q13.2 |
| 35143_at | family with sequence similarity 49, member A | FAM49A | 2p24.3-p24.2 |
| 35212_at | ring finger protein 139 | RNF139 | 8q24 |
| 35815_at | huntingtin interacting protein B | HYPB | 3p21.31 |
| 35928_at | thyroid peroxidase | TPO | 2p25 |
| 36244_at | zinc finger protein 239 | ZNF239 | 10q11.22-q11.23 |
| 36452_at | synaptopodin | SYNPO | 5q33.1 |
| 36548_at | KIAA0895 protein | KIAA0895 | 7p14.1 |
| 37348_s_at | high mobility group nucleosomal binding domain 3 | HMGN3 | 6q14.1 |
| 37360_at | lymphocyte antigen 6 complex, locus E | LY6E | 8q24.3 |
| 37436_at | sperm mitochondria-associated cysteine-rich protein | SMCP | 1q21.3 |
| 37801_at | ATPase, H+ transporting, lysosomal V0 subunit a isoform 2 | ATP6V0A2 | 12q24.31 |
| 37859_r_at | similar to 60S ribosomal protein L23a | LOC388574 | 17p13.3 |
| 39782_at | nuclear DNA-binding protein | C1D | 2p13-p12 |
| 39897_at | splicing factor YT521-B | YT521 | 4q13.2 |
| 40103_at | villin 2 (ezrin) | VIL2 | 6q25.2-q26 |
| 40451_at | polymerase (DNA directed), epsilon | POLE | 12q24.3 |
| 40470_at | oxoglutarate (alpha-ketoglutarate) dehydrogenase | OGDH | 7p14-p13 |
| | (lipoamide) | | |
| 40535_i_at | Eukaryotic translation initiation factor 5B | EIF5B | 2p11.1-q11.1 |
| 40885_s_at | syntaxin 16 | STX16 | 20q13.32 |
| 40982_at | hypothetical protein FLJ10534 | FLJ10534 | 17p13.3 |
| 41057_at | thioesterase superfamily member 2 | THEM2 | 6p22.2 |
| 41535_at | CDK2-associated protein 1 | CDK2AP1 | 12q24.31 |
| 41867_at | cAMP responsive element binding protein 3-like 1 | CREB3L1 | 11p11.2 |
| 425_at | interferon, alpha-inducible protein 27 | IFI27 | 14q32 |
| 428_s_at | beta-2-microglobulin | B2M | 15q21-q22.2 |
| 470_at | cell growth regulator with EF-hand domain 1 | CGREF1 | 2p23.3 |

ADRIAMYCIN PREDICTOR - Metagene 2

| | | | |
|---|---|---|---|
| 1050_at | melan-A | MLANA | 9p24.1 |
| 1109_s_at | platelet-derived growth factor alpha polypeptide | PDGFA | 7p22 |
| 1258_s_at | excision repair cross-complementing rodent repair | ERCC4 | 16p13.3-p13.11 |
| | deficiency, complementation group 4 | | |
| 1318_at | retinoblastoma binding protein 4 | RBBP4 | 1p35.1 |
| 1518_at | v-ets erythroblastosis virus E26 oncogene homolog 1 (avian) | ETS1 | 11q23.3 |
| 1536_at | CDC6 cell division cycle 6 homolog (S. cerevisiae) | CDC6 | 17q21.3 |
| 1847_s_at | B-cell CLL/lymphoma 2 | BCL2 | 18q21.33\|18q21.3 |
| 1909_at | B-cell CLL/lymphoma 2 | BCL2 | 18q21.33\|18q21.3 |
| 1910_s_at | B-cell CLL/lymphoma 2 | BCL2 | 18q21.33\|18q21.3 |
| 2010_at | S-phase kinase-associated protein 1A (p19A) | SKP1A | 5q31 |
| 2034_s_at | cyclin-dependent kinase inhibitor 1B (p27, Kip1) | CDKN1B | 12p13.1-p12 |
| 32138_at | dynamin 1 | DNM1 | 9q34 |
| 32167_at | peptidase (mitochondrial processing) beta | PMPCB | 7q22-q32 |
| 32611_at | prostatic binding protein | PBP | 12q24.23 |
| 32717_at | neuralized-like (Drosophila) | NEURL | 10q25.1 |
| 32820_at | CCR4-NOT transcription complex, subunit 4 | CNOT4 | 7q22-qter |
| 32966_at | apolipoprotein F | APOF | 12q13.3 |
| 33003_at | NCK adaptor protein 2 | NCK2 | 2q12 |
| 33239_at | hypothetical protein MGC33887 | MGC33887 | 17q24.2 |
| 33408_at | KIAA0934 | KIAA0934 | 10p15.3 |
| 33823_at | scavenger receptor class B, member 2 | SCARB2 | 4q21.1 |
| 33852_at | TIA1 cytotoxic granule-associated RNA binding protein | TIA1 | 2p13 |
| 33891_at | chloride intracellular channel 4 | CLIC4 | 1p36.11 |
| 33903_at | death-associated protein kinase 3 | DAPK3 | 19p13.3 |
| 33907_at | eukaryotic translation initiation factor 4 gamma, 3 | EIF4G3 | 1p36.12 |
| 33941_at | ADAM metallopeptidase domain 11 | ADAM11 | 17q21.3 |
| 33955_at | interleukin 12A (natural killer cell stimulatory factor 1, | IL12A | 3p12-q13.2 |
| | cytotoxic lymphocyte maturation factor 1, p35) | | |
| 34212_at | ATP/GTP binding protein 1 | AGTPBP1 | 9q21.33 |
| 34302_at | eukaryotic translation initiation factor 3, subunit 4 delta, | EIF3S4 | 19p13.2 |
| | 44 kDa | | |
| 34347_at | nuclear protein E3-3 | DKFZP564J0123 | 3p21.31 |
| 34858_at | potassium channel tetramerisation domain containing 2 | KCTD2 | 17q25.1 |
| 34884_at | carbamoyl-phosphate synthetase 1, mitochondrial | CPS1 | 2q35 |
| 34992g_at | sarcoglycan, delta (35 kDa dystrophin-associated | SGCD | 5q33-q34 |
| | glycoprotein) | | |
| 35279_at | Tax1 (human T-cell leukemia virus type I) binding protein 1 | TAX1BP1 | 7p15 |
| 35443_at | karyopherin alpha 6 (importin alpha 7) | KPNA6 | 1p35.1-p34.3 |
| 35680_r_at | dipeptidylpeptidase 6 | DPP6 | 7q36.2 |
| 35765_at | ADP-ribosylation factor related protein 1 | ARFRP1 | 20q13.3 |
| 35806_at | Golgi reassembly stacking protein 2, 55 kDa | GORASP2 | 2q31.1-q31.2 |
| 36132_at | aldehyde dehydrogenase 7 family, member A1 | ALDH7A1 | 5q31 |
| 36617_at | inhibitor of DNA binding 1, dominant negative helix-loop- | ID1 | 20q11 |
| | helix protein | | |
| 36794_at | zinc finger protein 250 | ZNF250 | 8q24.3 |
| 36827_at | acyl-Coenzyme A binding domain containing 3 | ACBD3 | 1q42.12 |
| 37326_at | proteolipid protein 2 (colonic epithelium-enriched) | PLP2 | Xp11.23 |
| 37344_at | major histocompatibility complex, class II, DM alpha | HLA-DMA | 6p21.3 |
| 37694_at | PHD finger protein 3 | PHF3 | 6q12 |
| 37742_at | galactosidase, beta 1 | GLB1 | 3p21.33 |
| 37748_at | KIAA0232 gene product | KIAA0232 | 4p16.1 |
| 37925_r_at | apolipoprotein M | APOM | 6p21.33 |
| 38003_s_at | diacylglycerol kinase, zeta 104 kDa | DGKZ | 11p11.2 |
| 38077_at | collagen, type VI, alpha 3 | COL6A3 | 2q37 |
| 38109_at | palmitoyl-protein thioesterase 2 | PPT2 | 6p21.3 |
| | EGF-like-domain, multiple 8 | EGFL8 | 6p21.32 |
| 38118_at | SHC (Src homology 2 domain containing) transforming | SHC1 | 1q21 |
| | protein 1 | | |
| 38121_at | tryptophanyl-tRNA synthetase | WARS | 14q32.31 |
| 38296_at | Trf (TATA binding protein-related factor)-proximal | TRFP | 6p21.1 |
| | homolog (Drosophila) | | |
| 38378_at | CD53 antigen | CD53 | 1p13 |
| 38652_at | chromosome 10 open reading frame 26 | C10orf26 | 10q24.32 |
| 39213_at | p21(CDKN1A)-activated kinase 7 | PAK7 | 20p12 |
| 39270_at | C-type lectin domain family 4, member M | CLEC4M | 19p13 |
| 39315_at | angiopoietin 1 | ANGPT1 | 8q22.3-q23 |
| 39385_at | alanyl (membrane) aminopeptidase (aminopeptidase N, | ANPEP | 15q25-q26 |
| | aminopeptidase M, microsomal aminopeptidase, CD13, | | |
| | p150) | | |
| 39800_s_at | HCLS1 associated protein X-1 | HAX1 | 1q21.3 |
| 40087_at | unc-13 homolog B (C. elegans) | UNC13B | 9p12-p11 |
| 40102_at | oxysterol binding protein-like 2 | OSBPL2 | 20q13.3 |
| 40201_at | dopa decarboxylase (aromatic L-amino acid decarboxylase) | DDC | 7p11 |
| 40433_at | glucosamine (N-acetyl)-6-sulfatase (Sanfilippo disease IIID) | GNS | 12q14 |
| 40567_at | tubulin, alpha 3 | TUBA3 | 12q12-12q14.3 |
| 40925_at | Pyruvate kinase, muscle | PKM2 | 15q22 |
| 41157_at | RAD23 homolog B (S. cerevisiae) | RAD23B | 9q31.2 |
| | similar to UV excision repair protein RAD23 homolog B | LOC131185 | 3p24.3 |
| | (HHR23B) (XP-C repair complementing complex 58 kDa | | |
| | protein) (P58) | | |
| 41293_at | Keratin 7 | KRT7 | 12q12-q13 |
| 41358_at | cyclin M2 | CNNM2 | 10q24.33 |
| 41377_f_at | UDP glucuronosyltransferase 2 family, polypeptide B7 | UGT2B7 | 4q13 |
| 41452_at | zinc finger protein 95 homolog (mouse) | ZFP95 | 7q22 |
| 41502_at | Homeodomain interacting protein kinase 3 | HIPK3 | 11p13 |
| 41609_at | major histocompatibility complex, class II, DM beta | HLA-DMB | 6p21.3 |
| 41643_at | SMA3 | SMA3 | 5q13 |
| | SMA5 | SMA5 | |
| 41838_at | 26S proteasome-associated UCH interacting protein 1 | UIP1 | Xq28 |
| 574_s_at | caspase 1, apoptosis-related cysteine peptidase (interleukin | CASP1 | 11q23 |
| | 1, beta, convertase) | | |
| 660_at | cytochrome P450, family 24, subfamily A, polypeptide 1 | CYP24A1 | 20q13 |
| 952_at | | | |
| 998_s_at | interleukin 1 receptor, type II | IL1R2 | 2q12-q22 |

CYTOXAN PREDICTOR - Metagene 3

| | | | |
|---|---|---|---|
| 1002_f_at | cytochrome P450, family 2, subfamily C, polypeptide 19 | CYP2C19 | 10q24.1-q24.3 |
| 1190_at | protein tyrosine phosphatase, receptor type, O | PTPRO | 12p13.3-p13.2\| |
| | | | 12p13-p12 |
| 1198_at | endothelin receptor type B | EDNRB | 13q22 |
| 1891_at | mitogen-activated protein kinase kinase kinase 8 | MAP3K8 | 10p11.23 |
| 1983_at | cyclin D2 | CCND2 | 12p13 |
| 200_at | bone morphogenetic protein 5 | BMP5 | 6p12.1 |
| 2037_s_at | ribosomal protein S6 kinase, 70 kDa, polypeptide 1 | RPS6KB1 | 17q23.2 |
| 31430_at | T cell receptor alpha variable 20 | TRAV20 | 14q11 |
| 31431_at | Fc fragment of IgG, receptor, transporter, alpha | FCGRT | 19q13.3 |
| 31719_at | fibronectin 1 | FN1 | 2q34 |
| 32339_at | pancreatic polypeptide | PPY | 17q21 |
| 32827_at | Sterol carrier protein 2 | SCP2 | 1p32 |
| 33132_at | cleavage and polyadenylation specific factor 1, 160 kDa | CPSF1 | 8q24.23 |
| 33673_r_at | UDP glucuronosyltransferase 2 family, polypeptide B17 | UGT2B17 | 4q13 |
| 34650_at | phosphodiesterase 3A, cGMP-inhibited | PDE3A | 12p12 |
| 34858_at | potassium channel tetramerisation domain containing 2 | KCTD2 | 17q25.1 |
| 36067_at | chemokine (C—C motif) ligand 19 | CCL19 | 9p13 |
| 36124_at | mercaptopyruvate sulfurtransferase | MPST | 22q13.1 |
| 36186_at | RNA binding protein S1, serine-rich domain | RNPS1 | 16p13.3 |
| 36207_at | SEC14-like 1 (S. cerevisiae) | SEC14L1 | 17q25.1-17q25.2 |
| 36652_at | uroporphyrinogen III synthase (congenital erythropoietic | UROS | 10q25.2-q26.3 |
| | porphyria) | | |
| 37363_at | metastasis suppressor 1 | MTSS1 | 8p22 |
| 38193_at | Immunoglobulin kappa variable 1-5 | IGKC | 2p12 |
| 38617_at | LIM domain kinase 2 | LIMK2 | 22q12.2 |
| 38783_at | mucin 1, transmembrane | MUC1 | 1q21 |
| 38788_at | promyelocytic leukemia | PML | 15q22 |
| | hypothetical protein LOC161527 | LOC161527 | 15q25.2 |
| 38795_s_at | upstream binding transcription factor, RNA polymerase I | UBTF | 17q21.3 |
| 39179_at | proteoglycan 2, bone marrow (natural killer cell activator, | PRG2 | 11q12 |
| | eosinophil granule major basic protein) | | |
| 40095_at | carbonic anhydrase II | CA2 | 8q22 |
| 40462_at | transient receptor potential cation channel, subfamily C, | TRPC4AP | 20q11.22 |
| | member 4 associated protein | | |
| 40513_at | protein phosphatase 3 (formerly 2B), regulatory subunit B, | PPP3R1 | 2p15 |
| | 19 kDa, alpha isoform (calcineurin B, type I) | | |
| 41183_at | cleavage stimulation factor, 3′ pre-RNA, subunit 3, 77 kDa | CSTF3 | 11p13 |
| 41307_at | hypothetical LOC400053 | LOC400053 | 12q15 |
| 41488_at | hypothetical protein A-211C6.1 | LOC57149 | 16p11.2 |
| 41722_at | nicotinamide nucleotide transhydrogenase | NNT | 5p13.1-5cen |

DOCETAXEL PREDICTOR - Metagene 4

| | | | |
|---|---|---|---|
| 1258_s_at | excision repair cross-complementing rodent repair | ERCC4 | 16p13.3-p13.11 |
| | deficiency, complementation group 4 | | |
| 141_s_at | BRF1 homolog, subunit of RNA polymerase III transcription | BRF1 | 14q |
| | initiation factor IIIB (S. cerevisiae) | | |
| 1566_at | neural cell adhesion molecule 1 | NCAM1 | 11q23.1 |
| 1751_g_at | phenylalanine-tRNA synthetase-like, alpha subunit | FARSLA | 19p13.2 |
| 1802_s_at | v-erb-b2 erythroblastic leukemia viral oncogene homolog 2, | ERBB2 | 17q11.2-q12\| |
| | neuro/glioblastoma derived oncogene homolog (avian) | | 17q21.1 |
| 1878_g_at | excision repair cross-complementing rodent repair | ERCC1 | 19q13.2-q13.3 |
| | deficiency, complementation group 1 (includes overlapping | | |
| | antisense sequence) | | |
| 1997_s_at | BCL2-associated X protein | BAX | 19q13.3-q13.4 |
| 2085_s_at | catenin (cadherin-associated protein), alpha 1, 102 kDa | CTNNA1 | 5q31 |
| 31431_at | Fc fragment of IgG, receptor, transporter, alpha | FCGRT | 19q13.3 |
| 31432_g_at | Fc fragment of IgG, receptor, transporter, alpha | FCGRT | 19q13.3 |
| 31638_at | NADH dehydrogenase (ubiquinone) Fe—S protein 7, 20 kDa | NDUFS7 | 19p13.3 |
| | (NADH-coenzyme Q reductase) | | |
| 32084_at | solute carrier family 22 (organic cation transporter), member 5 | SLC22A5 | 5q31 |
| 32099_at | scaffold attachment factor B2 | SAFB2 | 19p13.3 |
| 32217_at | chromosome 12 open reading frame 22 | C12orf22 | 12q13.11-q13.12 |
| 32237_at | KIAA0265 protein | KIAA0265 | 7q32.2 |
| 32331_at | adenylate kinase 3-like 1 | AK3L1 | 1p31.3 |
| 32523_at | clathrin, light polypeptide (Lcb) | CLTB | 4q2-q3\|5q35 |
| 32843_s_at | fibrillarin | FBL | 19q13.1 |
| 33047_at | BCL2-like 11 (apoptosis facilitator) | BCL2L11 | 2q13 |
| 33133_at | flightless I homolog (Drosophila) | FLII | 17p11.2 |
| 33203_s_at | forkhead box D1 | FOXD1 | 5q12-q13 |
| 33214_at | mitochondrial ribosomal protein S12 | MRPS12 | 19q13.1-q13.2 |
| 33285_i_at | hypothetical protein FLJ21168 | FLJ21168 | 1p13.1 |
| 33371_s_at | RAB31, member RAS oncogene family | RAB31 | 18p11.3 |
| 33387_at | growth arrest-specific 7 | GAS7 | 17p13.1 |
| 33443_at | serine incorporator 1 | SERINC1 | 6q22.31 |
| 34646_at | ribosomal protein S7 | RPS7 | 2p25 |
| 34772_at | coronin, actin binding protein, 2B | CORO2B | 15q23 |
| 34800_at | leucine-rich repeats and immunoglobulin-like domains 1 | LRIG1 | 3p14 |
| 34803_at | ubiquitin specific peptidase 12 | USP12 | 13q12.13 |
| 35017_f_at | HLA-G histocompatibility antigen, class I, G | HLA-G | 6p21.3 |
| 35654_at | phospholipase C, beta 4 | PLCB4 | 20p12 |
| 35713_at | Fanconi anemia, complementation group C | FANCC | 9q22.3 |
| 35769_at | G protein-coupled receptor 56 | GPR56 | 16q12.2-q21 |
| 35814_at | dendritic cell protein | hfl-B5 | 11p13 |
| 36208_at | bromodomain containing 2 | BRD2 | 6p21.3 |
| 36249_at | hypothetical protein LOC253982 | LOC253982 | 16p11.2 |
| 36394_at | lymphocyte antigen 6 complex, locus H | LY6H | 8q24.3 |
| 36527_at | RNA binding motif protein, X-linked 2 | RBMX2 | Xq25 |
| 36640_at | myosin, light polypeptide 2, regulatory, cardiac, slow | MYL2 | 12q23-q24.3 |
| 38662_at | Hypothetical protein FLJ38348 | FLJ38348 | 2p22.2 |
| 38830_at | ATP-binding cassette, sub-family F (GCN20), member 3 | ABCF3 | 3q27.1 |
| 39198_s_at | Tetratricopeptide repeat domain 15 | TTC15 | 2p25.2 |
| 40567_at | tubulin, alpha 3 | TUBA3 | 12q12-12q14.3 |
| 41062_at | polycomb group ring finger 1 | PCGF1 | 2p13.1 |
| 41076_at | gap junction protein, beta 3, 31 kDa (connexin 31) | GJB3 | 1p34 |
| 41284_at | Inositol polyphosphate-5-phosphatase, 40 kDa | INPP5A | 10q26.3 |
| 41688_at | plasma membrane proteolipid (plasmolipin) | PLLP | 16q13 |
| 41712_at | aquarius homolog (mouse) | AQR | 15q14 |
| 940_g_at | neurofibromin 1 (neurofibromatosis, von Recklinghausen | NF1 | 17q11.2 |
| | disease, Watson disease) | | |

ETOPOSIDE PREDICTOR - Metagene 5

| | | | |
|---|---|---|---|
| 1014_at | polymerase (DNA directed), gamma | POLG | 15q25 |
| 1187_at | ligase III, DNA, ATP-dependent | LIG3 | 17q11.2-q12 |
| 1232_s_at | insulin-like growth factor binding protein 1 | IGFBP1 | 7p13-p12 |
| 1455_f_at | cytochrome P450, family 2, subfamily C, polypeptide 9 | CYP2C9 | 10q24 |
| 159_at | vascular endothelial growth factor C | VEGFC | 4q34.1-q34.3 |
| 167_at | eukaryotic translation initiation factor 5 | EIF5 | 14q32.32 |
| 1703_g_at | E2F transcription factor 4, p107/p130-binding | E2F4 | 16q21-q22 |
| 1962_at | arginase, liver | ARG1 | 6q23 |
| 2046_at | | | |
| 295_s_at | | | |
| 296_at | | | |
| 310_s_at | microtubule-associated protein tau | MAPT | 17q21.1 |
| 31718_at | ATP-binding cassette, sub-family D (ALD), member 2 | ABCD2 | 12q11-q12 |
| 31719_at | fibronectin 1 | FN1 | 2q34 |
| 32377_at | IK cytokine, down-regulator of HLA II | IK | 2p15-p14 |
| 32386_at | MRNA full length insert cDNA clone EUROIMAGE | | |
| | 117929 | | |
| 32592_at | KIAA0323 | KIAA0323 | 14q11.2 |
| 33281_at | inhibitor of kappa light polypeptide gene enhancer in B-cells, | IKBKE | 1q32.1 |
| | kinase epsilon | | |
| 33447_at | myosin regulatory light chain MRCL3 | MRCL3 | 18p11.31 |
| 33903_at | death-associated protein kinase 3 | DAPK3 | 19p13.3 |
| 34319_at | S100 calcium binding protein P | S100P | 4p16 |
| 34347_at | nuclear protein E3-3 | DKFZP564J0123 | 3p21.31 |
| 34746_at | progestin and adipoQ receptor family member IV | PAQR4 | 16p13.3 |
| 34768_at | thioredoxin domain containing | TXNDC | 14q22.1 |
| 35275_at | carbonic anhydrase XII | CA12 | 15q22 |
| 35308_at | chromosome 9 open reading frame 74 | C9orf74 | 9q34.11 |
| 35443_at | karyopherin alpha 6 (importin alpha 7) | KPNA6 | 1p35.1-p34.3 |
| 35540_at | hyaluronoglucosaminidase 3 | HYAL3 | 3p21.3 |
| 35629_at | megakaryoblastic leukemia (translocation) 1 | MKL1 | 22q13 |
| 35668_at | receptor (calcitonin) activity modifying protein 1 | RAMP1 | 2q36-q37.1 |
| 35680_r_at | dipeptidylpeptidase 6 | DPP6 | 7q36.2 |
| 35734_at | ARP2 actin-related protein 2 homolog (yeast) | ACTR2 | 2p14 |
| 36096_at | chromosome 2 open reading frame 23 | C2orf23 | 2p11.2 |
| 36889_at | Fc fragment of IgE, high affinity I, receptor for; gamma | FCER1G | 1q23 |
| | polypeptide | | |
| 37933_at | retinoblastoma binding protein 6 | RBBP6 | 16p12.2 |
| 38220_at | dihydropyrimidine dehydrogenase | DPYD | 1p22 |
| 38481_at | replication protein A1, 70 kDa | RPA1 | 17p13.3 |
| 38758_at | PDGFA associated protein 1 | PDAP1 | 7q22.1 |
| 38759_at | butyrophilin, subfamily 3, member A2 | BTN3A2 | 6p22.1 |
| 39330_s_at | actinin, alpha 1 | ACTN1 | 14q24.1-q24.2\| |
| | | | 14q24\| |
| | | | 14q22-q24 |
| 39731_at | RNA binding motif protein, X-linked | RBMX | Xq26.3 |
| 39869_at | ElaC homolog 2 (E. coli) | ELAC2 | 17p11.2 |
| 40214_at | UDP-glucose ceramide glucosyltransferase | UGCG | 9q31 |
| 40224_s_at | SAPS domain family, member 2 | SAPS2 | 22q13.33 |
| 41358_at | cyclin M2 | CNNM2 | 10q24.33 |
| 41871_at | podoplanin | PDPN | 1p36.21 |
| 478_g_at | interferon regulatory factor 5 | IRF5 | 7q32 |
| 574_s_at | caspase 1, apoptosis-related cysteine peptidase (interleukin | CASP1 | 11q23 |
| | 1, beta, convertase) | | |
| 670_s_at | cAMP responsive element binding protein 5 | CREB5 | 7p15.1 |
| 902_at | EPH receptor B2 | EPHB2 | 1p36.1-p35 |

PACLITAXEL PREDICTOR - Metagene 6

| | | | |
|---|---|---|---|
| 1217_g_at | protein kinase C, beta 1 | PRKCB1 | 16p11.2 |
| 1258_s_at | excision repair cross-complementing rodent repair | ERCC4 | 16p13.3-p13.11 |
| | deficiency, complementation group 4 | | |
| 1586_at | insulin-like growth factor binding protein 3 | IGFBP3 | 7p13-p12 |
| 1802_s_at | v-erb-b2 erythroblastic leukemia viral oncogene homolog 2, | ERBB2 | 17q11.2-q12\| |
| | neuro/glioblastoma derived oncogene homolog (avian) | | 17q21.1 |
| 1823_g_at | | | |
| 1870_at | protein tyrosine phosphatase, non-receptor type 11 (Noonan | PTPN11 | 12q24 |
| | syndrome 1) | | |
| 1878_g_at | excision repair cross-complementing rodent repair | ERCC1 | 19q13.2-q13.3 |
| | deficiency, complementation group 1 (includes overlapping | | |
| | antisense sequence) | | |
| 1881_at | | | |
| 1902_at | excision repair cross-complementing rodent repair | ERCC1 | 19q13.2-q13.3 |
| | deficiency, complementation group 1 (includes overlapping | | |
| | antisense sequence) | | |
| 2000_at | ataxia telangiectasia mutated (includes complementation | ATM | 11q22-q23 |
| | groups A, C and D) | | |
| 32385_at | Rho-associated, coiled-coil containing protein kinase 1 | ROCK1 | 18q11.1 |
| 33047_at | BCL2-like 11 (apoptosis facilitator) | BCL2L11 | 2q13 |
| 33556_at | Huntingtin interacting protein E | HYPE | 12q24.1 |
| 34196_at | GATA zinc finger domain containing 1 | GATAD1 | 7q21-q22 |
| 34246_at | chromosome 6 open reading frame 145 | C6orf145 | 6p25.2 |
| 34470_at | transcription factor EC | TFEC | 7q31.2 |
| 34861_at | golgi autoantigen, golgin subfamily a, 3 | GOLGA3 | 12q24.33 |
| 34922_at | cadherin 19, type 2 | CDH19 | 18q22-q23 |
| 34983_at | Cytochrome P450, family 26, subfamily A, polypeptide 1 | CYP26A1 | 10q23-q24 |
| 35643_at | nucleobindin 2 | NUCB2 | 11p15.1-p14 |
| 35907_at | cyclin F | CCNF | 16p13.3 |
| 36519_at | excision repair cross-complementing rodent repair | ERCC1 | 19q13.2-q13.3 |
| | deficiency, complementation group 1 (includes overlapping | | |
| | antisense sequence) | | |
| 36594_s_at | exostoses (multiple) 2 | EXT2 | 11p12-p11 |
| 37377_i_at | lamin A/C | LMNA | 1q21.2-q21.3 |
| 37766_s_at | proteasome (prosome, macropain) 26S subunit, ATPase, 5 | PSMC5 | 17q23-q25 |
| 38702_at | polymerase (DNA directed), epsilon 3 (p17 subunit) | POLE3 | 9q33 |
| 39536_at | Homeo box (H6 family) 1 | HMX1 | 4p16.1 |
| 40359_at | Ras association (RalGDS/AF-6) domain family 7 | RASSF7 | 11p15.5 |
| 40528_at | LIM homeobox 2 | LHX2 | 9q33-q34.1 |
| 40567_at | tubulin, alpha 3 | TUBA3 | 12q12-12q14.3 |
| 40689_at | sel-1 suppressor of lin-12-like (C. elegans) | SEL1L | 14q24.3-q31 |
| 41044_at | WD repeat domain 67 | WDR67 | 8q24.13 |
| 41403_at | enolase 1, (alpha) | ENO1 | 1p36.3-p36.2 |
| | small nuclear ribonucleoprotein polypeptide F | SNRPF | 12q23.1 |
| 114_r_at | microtubule-associated protein tau | MAPT | 17q21.1 |
| 924_s_at | protein phosphatase 2 (formerly 2A), catalytic subunit, beta | PPP2CB | 8p12 |
| | isoform | | |

TOPOTECAN PREDICTOR - Metagene 7

| | | | |
|---|---|---|---|
| 1004_at | Burkitt lymphoma receptor 1, GTP binding protein | BLR1 | 11q23.3 |
| | (chemokine (C—X—C motif) receptor 5) | | |
| 1159_at | interleukin 7 | IL7 | 8q12-q13 |
| 1232_s_at | insulin-like growth factor binding protein 1 | IGFBP1 | 7p13-p12 |
| 1250_at | protein kinase, DNA-activated, catalytic polypeptide | PRKDC | 8q11 |
| 1256_at | protein tyrosine phosphatase, receptor type, D | PTPRD | 9p23-p24.3 |
| 1277_at | Rho guanine exchange factor (GEF) 16 | ARHGEF16 | 1p36.3 |
| 1367_f_at | ubiquitin C | UBC | 12q24.3 |
| 1384_at | protein phosphatase 2 (formerly 2A), regulatory subunit B | PPP2R2B | 5q31-5q32 |
| | (PR 52), beta isoform | | |
| 1490_at | v-myc myelocytomatosis viral oncogene homolog 1, lung | MYCL1 | 1p34.2 |
| | carcinoma derived (avian) | | |
| 1543_at | mitogen-activated protein kinase kinase 6 | MAP2K6 | 17q24.3 |
| 1562_g_at | dual specificity phosphatase 8 | DUSP8 | 11p15.5 |
| 1592_at | topoisomerase (DNA) II alpha 170 kDa | TOP2A | 17q21-q22 |
| 1599_at | cyclin-dependent kinase inhibitor 3 (CDK2-associated dual | CDKN3 | 14q22 |
| | specificity phosphatase) | | |
| 160043_at | v-myb myeloblastosis viral oncogene homolog (avian)-like 1 | MYBL1 | 8q22 |
| 1750_at | phenylalanine-tRNA synthetase-like, alpha subunit | FARSLA | 19p13.2 |
| 1782_s_at | stathmin 1/oncoprotein 18 | STMN1 | 1p36.1-p35 |
| 1827_s_at | v-myc myelocytomatosis viral oncogene homolog (avian) | MYC | 8q24.12-q24.13 |
| 1878_g_at | excision repair cross-complementing rodent repair | ERCC1 | 19q13.2-q13.3 |
| | deficiency, complementation group 1 (includes overlapping | | |
| | antisense sequence) | | |
| 1957_s_at | transforming growth factor, beta receptor I (activin A | TGFBR1 | 9q22 |
| | receptor type II-like kinase, 53 kDa) | | |
| 2041_i_at | v-abl Abelson murine leukemia viral oncogene homolog 1 | ABL1 | 9q34.1 |
| 2052_g_at | O-6-methylguanine-DNA methyltransferase | MGMT | 10q26 |
| 2055_s_at | integrin, beta 1 (fibronectin receptor, beta polypeptide, | ITGB1 | 10p11.2 |
| | antigen CD29 includes MDF2, MSK12) | | |
| 2056_at | fibroblast growth factor receptor 1 (fms-related tyrosine | FGFR1 | 8p11.2-p11.1 |
| | kinase 2, Pfeiffer syndrome) | | |
| 231_at | transglutaminase 2 (C polypeptide, protein-glutamine- | TGM2 | 20q12 |
| | gamma-glutamyltransferase) | | |
| 31520_at | chromobox homolog 2 (Pc class homolog, Drosophila) | CBX2 | 17q25.3 |
| 32097_at | pericentrin 2 (kendrin) | PCNT2 | 21q22.3 |
| 32115_r_at | adenosine A2a receptor | ADORA2A | 22q11.23 |
| 32259_at | enhancer of zeste homolog 1 (Drosophila) | EZH1 | 17q21.1-g21.3 |
| 32433_at | ribosomal protein L15 | RPL15 | 3p24.2 |
| 32528_at | ClpP caseinolytic peptidase, ATP-dependent, proteolytic | CLPP | 19p13.3 |
| | subunit homolog (E. coli) | | |
| 32530_at | tyrosine 3-monooxygenase/tryptophan 5-monooxygenase | YWHAQ | 2p25.1 |
| | activation protein, theta polypeptide | | |
| 32534_f_at | Vesicle-associated membrane protein 5 (myobrevin) | VAMP5 | 2p11.2 |
| 32605_r_at | RAB1A, member RAS oncogene family | RAB1A | 2p14 |
| 32606_at | Brain abundant, membrane attached signal protein 1 | BASP1 | 5p15.1-p14 |
| 32672_at | MRNA; cDNA DKFZp564M042 (from clone | | |
| | DKFZp564M042) | | |
| 32807_at | kelch repeat and BTB (POZ) domain containing 2 | KBTBD2 | 7p14.3 |
| 32811_at | myosin IC | MYO1C | 17p13 |
| 32846_s_at | kinectin 1 (kinesin receptor) | KTN1 | 14q22.1 |
| | protein disulfide isomerase family A, member 6 | PDIA6 | 2p25.1 |
| 33126_at | glycosyltransferase 8 domain containing 1 | GLT8D1 | 3p21.1 |
| 33327_at | chromosome 11 open reading frame 9 | C11orf9 | 11q12-q13.1 |
| 33336_at | Solute carrier family 4, anion exchanger, member 1 | SLC4A1 | 17q21-q22 |
| | (erythrocyte membrane protein band 3, Diego blood group) | | |
| 33403_at | chromosome 1 open reading frame 77 | C1orf77 | 1q21.3 |
| 33404_at | CAP, adenylate cyclase-associated protein, 2 (yeast) | CAP2 | 6p22.3 |
| 33439_at | SNF1-like kinase | SNF1LK | 21q22.3 |
| 33771_at | leucine rich repeat containing 8 family, member B | LRRC8B | 1p22.3 |
| 33784_at | TNF receptor-associated factor 2 | TRAF2 | 9q34 |
| 33786_r_at | glycine-, glutamate-, thienylcyclohexylpiperidine-binding | GlyBP | 1p36.32 |
| | protein | | |
| 33790_at | chemokine (C—C motif) ligand 14 | CCL14 | 17q11.2 |
| | chemokine (C—C motif) ligand 15 | CCL15 | |
| 33881_at | Acyl-CoA synthetase long-chain family member 3 | ACSL3 | 2q34-q35 |
| 338_at | activating transcription factor 6 | ATF6 | 1q22-q23 |
| 33993_at | myosin, light polypeptide 6, alkali, smooth muscle and non- | MYL6 | 12q13.2 |
| | muscle | | |
| 34090_at | | | |
| 34105_f_at | immunoglobulin heavy constant mu | IGHM | 14q32.33 |
| 34317_g_at | ribosomal protein S15a | RPS15A | 16p |
| 34319_at | S100 calcium binding protein P | S100P | 4p16 |
| 34374_g_at | HECT, UBA and WWE domain containing 1 | HUWE1 | Xp11.22 |
| 34794_r_at | plastin 3 (T isoform) | PLS3 | Xq23 |
| 34801_at | ubiquitin specific peptidase 52 | USP52 | 12q13.2-q13.3 |
| 34810_at | chromosome 16 open reading frame 49 | C16orf49 | 16q13 |
| 35129_at | sperm adhesion molecule 1 (PH-20 hyaluronidase, zona | SPAM1 | 7q31.3 |
| | pellucida binding) | | |
| 35263_at | eukaryotic translation initiation factor 4E binding protein 2 | EIF4EBP2 | 10q21-q22 |
| 35308_at | chromosome 9 open reading frame 74 | C9orf74 | 9q34.11 |
| 35365_at | integrin-linked kinase | ILK | 11p15.5-p15.4 |
| 35728_at | Uridine-cytidine kinase 1-like 1 | UCKL1 | 20q13.33 |
| 35750_at | likely ortholog of mouse immediate early response, | LEREPO4 | 2q32.1 |
| | erythropoietin 4 | | |
| 36118_at | nuclear receptor coactivator 1 | NCOA1 | 2p23 |
| 36148_at | amyloid beta (A4) precursor-like protein 1 | APLP1 | 19q13.1 |
| 36368_at | Clone 24479 mRNA sequence | | |
| 36524_at | Rho guanine nucleotide exchange factor (GEF) 4 | ARHGEF4 | 2q22 |
| 36549_at | solute carrier family 25 (mitochondrial carrier; peroxisomal | SLC25A17 | 22q13.2 |
| | membrane protein, 34 kDa), member 17 | | |
| 36576_at | H2A histone family, member Y | H2AFY | 5q31.3-q32 |
| 36637_at | annexin A11 | ANXA11 | 10q23 |
| 36658_at | 24-dehydrocholesterol reductase | DHCR24 | 1p33-p31.1 |
| 36789_f_at | leukocyte immunoglobulin-like receptor, subfamily B (with | LILRB5 | 19q13.4 |
| | TM and ITIM domains), member 5 | | |
| 36790_at | tropomyosin 1 (alpha) | TPM1 | 15q22.1 |
| 36791_g_at | tropomyosin 1 (alpha) | TPM1 | 15q22.1 |
| 36798_g_at | sialophorin (gpL115, leukosialin, CD43) | SPN | 16p11.2 |
| 36810_at | KIAA0485 protein | KIAA0485 | |
| 36884_at | CD163 antigen | CD163 | 12p13.3 |
| 36951_at | mitochondrial ribosomal protein L49 | MRPL49 | 11q13 |
| 36987_at | lamin B2 | LMNB2 | 19p13.3 |
| 37031_at | chromosome 9 open reading frame 10 | C9orf10 | 9q22.31 |
| 37321_at | tetratricopeptide repeat domain 1 | TTC1 | 5q32-q33.2 |
| 37407_s_at | myosin, heavy polypeptide 11, smooth muscle | MYH11 | 16p13.13-p13.12 |
| 37485_at | solute carrier family 27 (fatty acid transporter), member 2 | SLC27A2 | 15q21.2 |
| 37598_at | Ras association (RalGDS/AF-6) domain family 2 | RASSF2 | 20pter-p12.1 |
| 37699_at | methionyl aminopeptidase 2 | METAP2 | 12q22 |
| 37799_at | asialoglycoprotein receptor 2 | ASGR2 | 17p |
| 38112_g_at | chondroitin sulfate proteoglycan 2 (versican) | CSPG2 | 5q14.3 |
| 38124_at | midkine (neurite growth-promoting factor 2) | MDK | 11p11.2 |
| 38298_at | potassium large conductance calcium-activated channel, | KCNMB1 | 5q34 |
| | subfamily M, beta member 1 | | |
| 38337_at | zinc finger protein 193 | ZNF193 | 6p21.3 |
| 38393_at | KIAA0247 | KIAA0247 | 14q24.1 |
| 38395_at | NADH dehydrogenase (ubiquinone) Fe-S protein 1, 75 kDa | NDUFS1 | 2q33-q34 |
| | (NADH-coenzyme Q reductase) | | |
| 38432_at | interferon, alpha-inducible protein (clone IFI-15K) | G1P2 | 1p36.33 |
| 38448_at | actinin, alpha 2 | ACTN2 | 1q42-q43 |
| 38481_at | replication protein A1, 70 kDa | RPA1 | 17p13.3 |
| 38487_at | stabilin 1 | STAB1 | 3p21.1 |
| 38630_at | LAG1 longevity assurance homolog 6 (S. cerevisiae) | LASS6 | 2q24.3 |
| 38771_at | histone deacetylase 1 | HDAC1 | 1p34 |
| 38774_at | Syntaxin 7 | STX7 | 6q23.1 |
| 38841_at | ubiquitin associated domain containing 1 | UBADC1 | 9q34.3 |
| 38920_at | CHK1 checkpoint homolog (S. pombe) | CHEK1 | 11q24-q24 |
| 390_at | chemokine (C—C motif) receptor 4 | CCR4 | 3p24 |
| 39253_s_at | v-ral simian leukemia viral oncogene homolog A (ras | RALA | 7p15-p13 |
| | related) | | |
| 39276_g_at | calcium channel, voltage-dependent, L type, alpha 1D | CACNA1D | 3p14.3 |
| | subunit | | |
| 39326_at | ATPase, H+ transporting, lysosomal V0 subunit a isoform 1 | ATP6V0A1 | 17q21 |
| 39332_at | tubulin, beta polypeptide paralog | TUBB- | 6p25 |
| | | PARALOG | |
| 39408_at | acyl-Coenzyme A dehydrogenase, C-2 to C-3 short chain | ACADS | 12q22-qter |
| 39613_at | mannosidase, alpha, class 1A, member 1 | MAN1A1 | 6q22 |
| 39709_at | selenoprotein W, 1 | SEPW1 | 19q13.3 |
| 39866_at | ubiquitin specific peptidase 22 | USP22 | 17p11.2 |
| 39900_at | Immunoglobulin superfamily, member 4C | IGSF4C | 19q13.31 |
| 40022_at | Fukuyama type congenital muscular dystrophy (fukutin) | FCMD | 9q31-q33 |
| 40077_at | aconitase 1, soluble | ACO1 | 9p22-q32\| |
| | | | 9p22-p13 |
| 40095_at | carbonic anhydrase II | CA2 | 8q22 |
| 40170_at | Mannose-6-phosphate receptor binding protein 1 | M6PRBP1 | 19p13.3 |
| 40340_at | chromosome 6 open reading frame 162 | C6orf162 | 6q15-q16.1 |
| 40496_at | complement component 1, s subcomponent | C1S | 12p13 |
| 40563_at | | | |
| 40566_at | Protein kinase C, alpha | PRKCA | 17q22-q23.2 |
| 40641_at | BTAF1 RNA polymerase II, B-TFIID transcription factor- | BTAF1 | 10q22-q23 |
| | associated, 170 kDa (Mot1 homolog, S. cerevisiae) | | |
| 40691_at | zinc finger protein 274 | ZNF274 | 19qter |
| 40780_at | C-terminal binding protein 2 | CTBP2 | 10q26.13 |
| 40935_at | hypothetical protein MGC11308 | MGC11308 | 12q13.13 |
| 41196_at | Karyopherin (importin) beta 1 | KPNB1 | 17q21.32 |
| 41222_at | signal transducer and activator of transcription 6, interleukin- | STAT6 | 12q13 |
| | 4 induced | | |
| 41235_at | activating transcription factor 4 (tax-responsive enhancer | ATF4 | 22q13.1 |
| | element B67) | | |
| 41272_s_at | Matrix-remodelling associated 7 | TMAP1 | 17q25.1 |
| 41294_at | keratin 7 | KRT7 | 12q12-q13 |
| 41353_at | tumor necrosis factor receptor superfamily, member 17 | TNFRSF17 | 16p13.1 |
| 41477_at | potassium inwardly-rectifying channel, subfamily J, member | KCNJ13 | 2q37 |
| | 13 | | |
| 41543_at | AF4/FMR2 family, member 3 | AFF3 | 2q11.2-q12 |
| 41666_at | heat shock 70 kDa protein 12A | HSPA12A | |
| 41737_at | serine/arginine repetitive matrix 1 | SRRM1 | 1p36.11 |
| 41743_i_at | optineurin | OPTN | 10p13 |
| 41744_at | optineurin | OPTN | 10p13 |
| 41871_at | podoplanin | PDPN | 1p36.21 |
| 423_at | Ewing sarcoma breakpoint region 1 | EWSR1 | 22q12.2 |
| 464_s_at | interferon-induced protein 35 | IFI35 | 17q21 |
| 547_s_at | nuclear receptor subfamily 4, group A, member 2 | NR4A2 | 2q22-q23 |
| 580_at | histone 1, H1e | HIST1H1E | 6p21.3 |
| 627_g_at | arginine vasopressin receptor 1B | AVPR1B | 1q32 |
| 671_at | secreted protein, acidic, cysteine-rich (osteonectin) | SPARC | 5q31.3-q32 |
| 866_at | thrombospondin 1 | THBS1 | 15q15 |
| 874_at | chemokine (C—C motif) ligand 2 | CCL2 | 17q11.2-q21.1 |
| 883_s_at | pim-1 oncogene | PIM1 | 6p21.2 |
| 884_at | integrin, alpha 3 (antigen CD49C, alpha 3 subunit of VLA-3 | ITGA3 | 17q21.33 |
| | receptor) | | |
| 889_at | integrin, beta 8 | ITGB8 | 7p21.1 |
| 918_at | | | |

TABLE 6

| | | Genomic-based |
|---|---|---|
| | Actual Overall | Prediction of Response |
| Tumor data set/Response | response | (i.e. PPV for Response) |
| Breast Tumor Data | | |
| MDACC | 13/51 (25.4%) | 11/13 (85.7%) |
| Adjuvant | 33/45 (66.6%) | 28/31 (90.3%) |
| Neoadjuvant Docetaxel | 13/24 (54.1%) | 11/13 (85.7%) |
| Ovarian | | |
| Topotecan | 20/48 (41.6%) | 17/22 (77.3%) |
| Paclitaxel | 20/35 (57.1%) | 20/28 (71.5%) |
| Docetaxel | 7/14 (50%) | 6/7 (85.7%) |
| Adriamycin (Evans et al) | 24/122 (19.6%) | 19/33 (57.5%) |

TABLE 7

| Validations/Drugs | Topotecan | Adriamycin | Etoposide | 5-Flourouracil | Paclitaxel | Cytoxan | Docetaxel | |
|---|---|---|---|---|---|---|---|---|
| In vitro Data | | | | | | | | |
| Accuracy | 18/20 (90%) | 18/25 (86%) | 21/24 (87%) | 21/24 (87%) | 26/28 (92.8%) | 25/29 (86.2%) | P < 0.001** | |
| PPV | 12/14 (86%) | 13/13 (100%) | 6/8 (75%) | 14/14 (100%) | 21/21 (100%) | 13/15 (86.6%) | | |
| NPV | 6/6 (100%) | 5/8 (62.5%) | 15/16 (94%) | 7/10 (70%) | 5/7 (71.5%) | 12/14 (86%) | | |
| | | | | | | | Breast | Ovarian |
| In vivo | | | | | | | | |
| (Patient) Data | | | | | | | | |
| Accuracy | 40/48 (83.32%) | 99/122 (81%) | — | — | 28/35 (80%) | — | 22/24 (91.6%) | 12/14 |
| | | | | | | | | (85.7%) |
| PPV | 17/22 (77.34%) | 19/33 (57.5%) | | | 20/28 (71.4%) | | 11/13 (85.7%) | 6/7 |
| | | | | | | | | (85.7%) |
| NPV | 23/26 (88.5%) | 80/89 (89.8%) | | | 7/7 (100%) | | 11/11 (100%) | 6/7 |
| | | | | | | | | (85.7%) |

PPV—positive predictive value,
NPV—negative predictive value.
**Determining accuracy for the docetaxel predictor in the LJC cell line data set was not possible since docetaxel was not one of the drugs studied. Instead, the docetaxel predictor was validated in two independent cell line experiments, correlating predicted probability of response to docetaxel in vitro with actual IC50 of docetaxel by cell line (FIG. 1C).

TABLE 8

| | | | Genomic predictor of | |
|---|---|---|---|---|
| | Docetaxel | Docetaxel | response to | Predictor of response to |
| | predictor | predictor | TFAC chemotherapy | TFAC chemotherapy |
| Validations/Predictors | (Potti et al) | (Chang et al)** | (Potti et al) | (Pusztai et al)** |
| Breast neoadjuvant | | | | |
| data (Chang et al) | | | | |
| Accuracy | 22/24 (91.6%) | 87.5% | | |
| PPV | 11/13 (85.7%) | 92% | | |
| NPV | 11/11 (100%) | 83% | | |
| AUC of ROC | 0.97 | 0.96 | | |
| MDACC data | | | | |
| (Pusztai et al) | | | | |
| Accuracy | | | 42/51 (82.3%) | 74% |
| PPV | | | 11/18 (61.1%) | 44% |
| NPV | | | 31/33 (94%) | 93% |

PPV—positive predictive value,
NPV—negative predictive value.
**For both the Chang and Pusztai data, the actual numbers of predicted responders was not available, just the predictive accuracies. Also, the predictive accuracy reported for the Chang data is not in an independent validation, instead it is for a leave-one out cross validation.

TABLE 9
Genes constituting the PI3 kinase predictor

| Gene Symbol | Affymetrix Probe ID | Gene Title |
|---|---|---|
| RFC2 | 1053_at | replication factor C (activator 1) 2, 40 kDa |
| KIAA0153 | 1552257_a_at | KIAA0153 protein |
| EXOSC6 | 1553947_at | exosome component 6 |
| RHOB | 1553962_s_at | ras homolog gene family, member B |
| MAD2L1 | 1554768_a_at | MAD2 mitotic arrest deficient-like 1 (yeast) |
| RBM15 | 1555762_s_at | RNA binding motif protein 15 |
| SPEN | 1556059_s_at | spen homolog, transcriptional regulator (Drosophila) |
| C6orf150 | 1559051_s_at | chromosome 6 open reading frame 150 |
| HSPA1A | 200799_at | heat shock 70 kDa protein 1A |
| HSPA1A///HSPA1B | 200800_s_at | heat shock 70 kDa protein 1A/// heat shock 70 kDa protein 1B |
| NOL5A | 200875_s_at | nucleolar protein 5A (56 kDa with KKE/D repeat) |
| CSE1L | 201112_s_at | CSE1 chromosome segregation 1-like (yeast) |
| PCNA | 201202_at | proliferating cell nuclear antigen |
| JUN | 201464_x_at | v-jun sarcoma virus 17 oncogene homolog (avian) |
| JUN | 201465_s_at | v-jun sarcoma virus 17 oncogene homolog (avian) |
| JUN | 201466_s_at | v-jun sarcoma virus 17 oncogene homolog (avian) |
| JUNB | 201473_at | jun B proto-oncogene |
| MCM3 | 201555_at | MCM3 minichromosome maintenance deficient 3 (S. cerevisiae) |
| EGR1 | 201693_s_at | early growth response 1 |
| DNMT1 | 201697_s_at | DNA (cytosine-5-)-methyltransferase 1 |
| MCM5 | 201755_at | MCM5 minichromosome maintenance deficient 5, cell division cycle 46 (S. cerevisiae) |
| RRM2 | 201890_at | ribonucleotide reductase M2 polypeptide |
| MCM6 | 201930_at | MCM6 minichromosome maintenance deficient 6 (MIS5 homolog, S. pombe) (S. cerevisiae) |
| NASP | 201970_s_at | nuclear autoantigenic sperm protein (histone-binding) |
| SPEN | 201997_s_at | spen homolog, transcriptional regulator (Drosophila) |
| IER2 | 202081_at | immediate early response 2 |
| MCM2 | 202107_s_at | MCM2 minichromosome maintenance deficient 2, mitotin (S. cerevisiae) |
| MTHFD1 | 202309_at | methylenetetrahydrofolate dehydrogenase (NADP+ dependent) 1, methenyltetrahydrofolate |
| | | cyclohydrolase, formyltetrahydrofolate synthetase |
| UNG | 202330_s_at | uracil-DNA glycosylase |
| HSPA1B | 202581_at | heat shock 70 kDa protein 1B |
| MSH6 | 202911_at | mutS homolog 6 (E. coli) |
| SSX2IP | 203017_s_at | synovial sarcoma, X breakpoint 2 interacting protein |
| RNASEH2A | 203022_at | ribonuclease H2, large subunit |
| PEX5 | 203244_at | peroxisomal biogenesis factor 5 |
| LMNB1 | 203276_at | lamin B1 |
| POLD1 | 203422_at | polymerase (DNA directed), delta 1, catalytic subunit 125 kDa |
| CDC6 | 203968_s_at | CDC6 cell division cycle 6 homolog (S. cerevisiae) |
| ZWINT | 204026_s_at | ZW10 interactor |
| CDC45L | 204126_s_at | CDC45 cell division cycle 45-like (S. cerevisiae) |
| RFC3 | 204128_s_at | replication factor C (activator 1) 3, 38 kDa |
| POLA2 | 204441_s_at | polymerase (DNA directed), alpha 2 (70 kD subunit) |
| CDC7 | 204510_at | CDC7 cell division cycle 7 (S. cerevisiae) |
| D1PA | 204610_s_at | hepatitis delta antigen-interacting protein A |
| ACD | 204617_s_at | adrenocortical dysplasia homolog (mouse) |
| CDC25A | 204695_at | cell division cycle 25A |
| FEN1 | 204767_s_at | flap structure-specific endonuclease 1 |
| FEN1 | 204768_s_at | flap structure-specific endonuclease 1 |
| MYB | 204798_at | v-myb myeloblastosis viral oncogene homolog (avian) |
| TOP3A | 204946_s_at | topoisomerase (DNA) III alpha |
| DDX10 | 204977_at | DEAD (Asp-Glu-Ala-Asp) box polypeptide 10 |
| RAD51 | 205024_s_at | RAD51 homolog (RecA homolog, E. coli) (S. cerevisiae) |
| CCNE2 | 205034_at | cyclin E2 |
| PRIM1 | 205053_at | primase, polypeptide 1, 49 kDa |
| BARD1 | 205345_at | BRCA1 associated RING domain 1 |
| CHEK1 | 205393_s_at | CHK1 checkpoint homolog (S. pombe) |
| H2AFX | 205436_s_at | H2A histone family, member X |
| FLJ12973 | 205519_at | hypothetical protein FLJ12973 |
| GEMIN4 | 205527_s_at | gem (nuclear organelle) associated protein 4 |
| SLBP | 206052_s_at | stem-loop (histone) binding protein |
| KIAA0186 | 206102_at | KIAA0186 gene product |
| AKR7A3 | 206469_x_at | aldo-keto reductase family 7, member A3 (aflatoxin aldehyde reductase) |
| TLE3 | 206472_s_at | transducin-like enhancer of split 3 (E(sp1) homolog, Drosophila) |
| GADD45B | 207574_s_at | growth arrest and DNA-damage-inducible, beta |
| PRPS1 | 208447_s_at | phosphoribosyl pyrophosphate synthetase 1 |
| BRD2 | 208685_x_at | bromodomain containing 2 |
| BRD2 | 208686_s_at | bromodomain containing 2 |
| MCM7 | 208795_s_at | MCM7 minichromosome maintenance deficient 7 (S. cerevisiae) |
| ID1 | 208937_s_at | inhibitor of DNA binding 1, dominant negative helix-loop-helix protein |
| GADD45B | 209304_x_at | growth arrest and DNA-damage-inducible, beta |
| GADD45B | 209305_s_at | growth arrest and DNA-damage-inducible, beta |
| POLR1C | 209317_at | polymerase (RNA) I polypeptide C, 30 kDa |
| PRKRIR | 209323_at | protein-kinase, interferon-inducible double stranded RNA dependent inhibitor, |
| | | repressor of (P58 repressor) |
| MSH2 | 209421_at | mutS homolog 2, colon cancer, nonpolyposis type 1 (E. coli) |
| PPAT | 209433_s_at | phosphoribosyl pyrophosphate amidotransferase |
| PPAT | 209434_s_at | phosphoribosyl pyrophosphate amidotransferase |
| PRPS1 | 209440_at | phosphoribosyl pyrophosphate synthetase 1 |
| RPA3 | 209507_at | replication protein A3, 14 kDa |
| EED | 209572_s_at | embryonic ectoderm development |
| GAS2L1 | 209729_at | growth arrest-specific 2 like 1 |
| RRM2 | 209773_s_at | ribonucleotide reductase M2 polypeptide |
| SLC19A1 | 209777_s_at | solute carrier family 19 (folate transporter), member 1 |
| CDT1 | 209832_s_at | DNA replication factor |
| SHMT1 | 209980_s_at | serine hydroxymethyltransferase 1 (soluble) |
| TAF5 | 210053_at | TAF5 RNA polymerase II, TATA box binding protein (TBP)-associated factor, 100 kDa |
| MCM7 | 210983_s_at | MCM7 minichromosome maintenance deficient 7 (S. cerevisiae) |
| MSH6 | 211450_s_at | mutS homolog 6 (E. coli) |
| CCNE2 | 211814_s_at | cyclin E2 |
| RHOB | 212099_at | ras homolog gene family, member B |
| MCM4 | 212141_at | MCM4 minichromosome maintenance deficient 4 (S. cerevisiae) |
| MCM4 | 212142_at | MCM4 minichromosome maintenance deficient 4 (S. cerevisiae) |
| KCTD12 | 212188_at | potassium channel tetramerisation domain containing 12///potassium channel |
| | | tetramerisation domain containing 12 |
| KCTD12 | 212192_at | potassium channel tetramerisation domain containing 12 |
| MAC30 | 212281_s_at | hypothetical protein MAC30 |
| POLD3 | 212836_at | polymerase (DNA-directed), delta 3, accessory subunit |
| KIAA0406 | 212898_at | KIAA0406 gene product |
| FLJI0719 | 213007_at | hypothetical protein FLJI0719 |
| ITPKC | 213076_at | inositol 1, 4, 5-trisphosphate 3-kinase C |
| ZNF473 | 213124_at | zinc finger protein 473 |
| — | 213281_at | — |
| CCNE1 | 213523_at | cyclin E1 |
| GADD45B | 213560_at | Growth arrest and DNA-damage-inducible, beta |
| GAL | 214240_at | galanin |
| BRD2 | 214911_s_at | bromodomain containing 2 |
| UMPS | 215165_x_at | uridine monophosphate synthetase (orotate phosphoribosyl transferase and |
| | | orotidine-5′-decarboxylase) |
| MCM5 | 216237_s_at | MCM5 minichromosome maintenance deficient 5, cell division cycle 46 (S. cerevisiae) |
| LMNB2 | 216952_s_at | lamin B2 |
| GEMIN4 | 217099_s_at | gem (nuclear organelle) associated protein 4 |
| SUPT16H | 217815_at | suppressor of Ty 16 homolog (S. cerevisiae) |
| GMNN | 218350_s_at | geminin, DNA replication inhibitor |
| RAMP | 218585_s_at | RA-regulated nuclear matrix-associated protein |
| SLC25A15 | 218653_at | solute carrier family 25 (mitochondrial carrier; ornithine transporter) member 15 |
| FLJ13912 | 218719_s_at | hypothetical protein FLJ13912 |
| ATAD2 | 218782_s_at | ATPase family, AAA domain containing 2 |
| C10orf117 | 218889_at | chromosome 10 open reading frame 117 |
| MGC10993 | 218897_at | hypothetical protein MGC10993 |
| C21orf45 | 219004_s_at | chromosome 21 open reading frame 45 |
| RPP25 | 219143_s_at | ribonuclease P 25 kDa subunit |
| FLJ20516 | 219258_at | timeless-interacting protein |
| MGC4504 | 219270_at | hypothetical protein MGC4504 |
| RBM15 | 219286_s_at | RNA binding motif protein 15 |
| FLJ11078 | 219354_at | hypothetical protein FLJ11078 |
| DCLRE1B | 219490_s_at | DNA cross-link repair 1B (PSO2 homolog, S. cerevisiae) |
| FLJ34077 | 219731_at | weakly similar to zinc finger protein 195 |
| FLJ20257 | 219798_s_at | hypothetical protein FLJ20257 |
| MCM10 | 220651_s_at | MCM10 minichromosome maintenance deficient 10 (S. cerevisiae) |
| TBRG4 | 220789_s_at | transforming growth factor beta regulator 4 |
| Pfs2 | 221521_s_at | DNA replication complex GINS protein PSF2 |
| LEF1 | 221558_s_at | lymphoid enhancer-binding factor 1 |
| ZNF45 | 222028_at | zinc finger protein 45 |
| MCM4 | 222036_s_at | MCM4 minichromosome maintenance deficient 4 (S. cerevisiae) |
| MCM4 | 222037_at | MCM4 minichromosome maintenance deficient 4 (S. cerevisiae) |
| CASP8AP2 | 222201_s_at | CASP8 associated protein 2 |
| MGC4692 | 222622_at | Hypothetical protein MGC4692 |
| RAMP | 222680_s_at | RA-regulated nuclear matrix-associated protein |
| FIGNL1 | 222843_at | fidgetin-like 1 |
| SLC25A19 | 223222_at | solute carrier family 25 (mitochondrial deoxynucleotide carrier), member 19 |
| UBE2T | 223229_at | ubiquitin-conjugating enzyme E2T (Putative) |
| TCF19 | 223274_at | transcription factor 19 (SC1) |
| PDXP | 223290_at | pyridoxal (pyridoxine, vitamin B6) phosphatase |
| POLR1B | 223403_s_at | polymerase (RNA) I polypeptide B, 128 kDa |
| ANKRD32 | 223542_at | ankyrin repeat domain 32 |
| IL17RB | 224361_s_at | interleukin 17 receptor B///interleukin 17 receptor B |
| CDCA7 | 224428_s_at | cell division cycle associated 7///cell division cycle associated 7 |
| MGC13096 | 224467_s_at | hypothetical protein MGC13096///hypothetical protein MGC13096 |
| CDCA5 | 224753_at | cell division cycle associated 5 |
| TMEM18 | 225489_at | transmembrane protein 18 |
| MGC20419 | 225642_at | hypothetical protein BC012173 |
| UHRF1 | 225655_at | ubiquitin-like, containing PHD and RING finger domains, 1 |
| — | 225716_at | Full-length cDNA clone CS0DK008Y109 of HeLa cells Cot 25-normalized |
| | | of Homo sapiens (human) |
| MGC23280 | 226121_at | hypothetical protein MGC23280 |
| C13orf8 | 226194_at | chromosome 13 open reading frame 8 |
| — | 226832_at | Hypothetical LOC389188 |
| EGR1 | 227404_s_at | Early growth response 1 |
| ZMYND19 | 227477_at | zinc finger, MYND domain containing 19 |
| BARD1 | 227545_at | BRCA1 associated RING domain 1 |
| KIAA1393 | 227653_at | KIAA1393 |
| GPR27 | 227769_at | G protein-coupled receptor 27 |
| RP13-15M17.2 | 228671_at | Novel protein |
| IL17D | 228977_at | Interleukin 17D |
| JPH1 | 229139_at | junctophilin 1 |
| ZNF367 | 229551_x_at | zinc finger protein 367 |
| MGC35521 | 235431_s_at | pellino 3 alpha |
| — | 239312_at | Transcribed locus |
| CSPG5 | 39966_at | chondroitin sulfate proteoglycan 5 (neuroglycan C) |

What is claimed is:

1. A method for identifying whether an individual with ovarian cancer will be responsive to a platinum-based therapy comprising:
a. Obtaining a cellular sample from the individual;
b. Analyzing said sample to obtain a first gene expression profile;
c. Comparing said first gene expression profile to a platinum chemotherapy responsivity predictor set of gene expression profiles; and
d. Identifying whether said individual will be responsive to a platinum-based therapy.

2. The method of claim 1 wherein the cellular sample is taken from a tumor sample.

3. The method of claim 1 wherein the cellular sample is taken from ascites.

4. The method of claim 1 wherein the nucleic acids contained within the cellular sample are used to obtain a first gene expression profile.

5. The method of claim 1 wherein the platinum chemotherapy responsivity predictor set of gene expression profiles comprises at least 5 genes from Table 2.

6. The method of claim 1 wherein the platinum chemotherapy responsivity predictor set of gene expression profiles comprises at least 10 genes from Table 2.

7. The method of claim 1 wherein the platinum chemotherapy responsivity predictor set of gene expression profiles comprises at least 15 genes from Table 2.

8. The method of claim 1 wherein the individual is identified in step (d) as a complete responder by complete disappearance of all measurable and assessable disease or, in the absence of measurable lesions, a normalization of the CA-125 level following adjuvant therapy.

9. The method of claim 1 wherein the individual is identified in step (d) as an incomplete responder comprising partial responders, having stable disease, or demonstrating progressive disease during primary therapy.

10. The method of claim 1 wherein the platinum-based therapy is selected from the group consisting of cisplatin, carboplatin, oxaliplatin and nedaplatin.

11. The method of claim 10 wherein a taxane is additionally administered.

12. A method of identifying whether an individual will benefit from the administration of an additional cancer therapeutic other than a platinum-based therapeutic comprising:
a. Obtaining a cellular sample from the individual;
b. Analyzing said sample to obtain a first gene expression profile;
c. Comparing said first gene expression profile to a platinum chemotherapy responsivity predictor set of gene expression profiles to identify whether said individual will be responsive to a platinum-based therapy;
d. If said individual is an incomplete responder to platinum based therapy, then comparing the first gene expression profile to a set of gene expression profiles that is capable of predicting responsiveness to other cancer therapy agents;
thereby identifying whether said individual would benefit from the administration of one or more cancer therapy agents.

13. The method of claim 12 wherein the cellular sample is taken from a tumor sample.

14. The method of claim 12 wherein the cellular sample is taken from ascites.

15. The method of claim 12 wherein the set of gene expression profiles that is capable of predicting responsiveness to salvage therapy agents comprises at least 5 genes from Table 5.

16. The method of claim 12 wherein the set of gene expression profiles that is capable of predicting responsiveness to salvage therapy agents comprises at least 10 genes from Table 5.

17. The method of claim 12 wherein the set of gene expression profiles that is capable of predicting responsiveness to salvage therapy agents comprises at least 15 genes from Table 5.

18. The method of claim 12 wherein the additional cancer therapy agent is a salvage therapy agent.

19. The method of claim 18 wherein the salvage therapy agent is selected from the group consisting of topotecan, adriamycin, doxorubicin, cytoxan, cyclophosphamide, gemcitabine, etoposide, ifosfamide, paclitaxel, docetaxel, and taxol.

20. The method of claim 12 wherein the additional cancer therapy agent targets a signal transduction pathway that is deregulated.

21. The method of claim 20 wherein the additional cancer therapy agent is selected from the group consisting of inhibitors of the Src pathway, inhibitors of the E2F3 pathway, inhibitors of the Myc pathway, and inhibitors of the beta-catenin pathway.

22. A method of treating an individual with ovarian cancer comprising:
a. Obtaining a cellular sample from the individual;
b. Analyzing said sample to obtain a first gene expression profile;
c. Comparing said first gene expression profile to a platinum chemotherapy responsivity predictor set of gene expression profiles to identify whether said individual will be responsive to a platinum-based therapy;
d. If said individual is a complete responder or incomplete responder, then administering an effective amount of platinum-based therapy to the individual;
e. If said individual is predicted to be an incomplete responder to platinum based therapy, then comparing the first gene expression profile to a set of gene expression profiles that is predictive of responsivity to additional cancer therapeutics to identify to which additional cancer therapeutic the individual would be responsive; and
f. Administering to said individual an effective amount of one or more of the additional cancer therapeutic that was identified in step (e);
thereby treating the individual with ovarian cancer.

23. The method of claim 22 wherein the cellular sample is taken from a tumor sample.

24. The method of claim 22 wherein the cellular sample is taken from ascites.

25. The method of claim 22 wherein the set of gene expression profiles that is capable of predicting responsiveness to salvage therapy agents comprises at least 5 genes from Table 4 or Table 5.

26. The method of claim 22 wherein the set of gene expression profiles that is capable of predicting responsiveness to salvage therapy agents comprises at least 10 genes from Table 4 or Table 5.

27. The method of claim 22 wherein the set of gene expression profiles that is capable of predicting responsiveness to salvage therapy agents comprises at least 15 genes from Table 4 or Table 5.

28. The method of claim 22 wherein the additional cancer therapeutic is a salvage agent.

29. The method of claim 28 wherein the salvage therapy agent is selected from the group consisting of topotecan, adriamycin, doxorubicin, cytoxan, cyclophosphamide, gemcitabine, paclitaxel, docetaxel, and taxol.

30. The method of claim 22 wherein the additional cancer therapy agent targets a signal transduction pathway that is deregulated.

31. The method of claim 30 wherein the additional cancer therapy agent is selected from the group consisting of inhibitors of the Src pathway, inhibitors of the E2F3 pathway, inhibitors of the Myc pathway, and inhibitors of the beta-catenin pathway.

32. The method of claim 22 wherein the platinum-based therapy is administered first, followed by the administration of one or more salvage therapy agent.

33. The method of claim 22 wherein the platinum-based therapy is administered concurrently with one or more salvage therapy agent.

34. The method of claim 22 wherein one or more salvage therapy agent is administered by itself.

35. The method of claim 22 wherein the salvage therapy agent is administered first, followed by the administration of one or more platinum-based therapy.

36. A method of reducing toxicity of chemotherapeutic agents in an individual with cancer comprising:
a. Obtaining a cellular sample from the individual;
b. Analyzing said sample to obtain a first gene expression profile;
c. Comparing said first gene expression profile to a set of gene expression profiles that is capable of predicting responsiveness to common chemotherapeutic agents; and
d. Administering to the individual an effective amount of that agent.

37. A gene chip for predicting an individual's responsivity to a platinum-based therapy comprising the gene expression profile of at least 5 genes selected from Table 2.

38. A gene chip for predicting an individual's responsivity to a platinum-based therapy comprising the gene expression profile of at least 10 genes selected from Table 2.

39. A gene chip for predicting an individual's responsivity to a platinum-based therapy comprising the gene expression profile of at least 20 genes selected from Table 2.

40. A kit comprising a gene chip of any one of claims 37 to 39 and a set of instructions for determining an individual's responsivity to platinum-based chemotherapy agents.

41. A gene chip for predicting an individual's responsivity to a salvage therapy agent comprising the gene expression profile of at least 5 genes selected from Table 4 or Table 5.

42. A gene chip for predicting an individual's responsivity to a salvage therapy agent comprising the gene expression profile of at least 10 genes selected from Table 4 or Table 5.

43. A gene chip for predicting an individual's responsivity to a salvage therapy agent comprising the gene expression profile of at least 20 genes selected from Table 4 or Table 5.

44. A kit comprising a gene chip of any one of claims 41 to 43 and a set of instructions for determining an individual's responsivity to salvage therapy agents.

45. A computer readable medium comprising gene expression profiles comprising at least 5 genes from any of Tables 2, 3 or 4.

46. A computer readable medium comprising gene expression profiles comprising at least 15 genes from Tables 2, 3 or 4.

47. A computer readable medium comprising gene expression profiles comprising at least 25 genes from Tables 2, 3 or 4.

48. A method for estimating the efficacy of a therapeutic agent in treating a subject afflicted with cancer, the method comprising:
a. Determining the expression level of multiple genes in a tumor biopsy sample from the subject;
b. Defining the value of one or more metagenes from the expression levels of step (a), wherein each metagene is defined by extracting a single dominant value using singular value decomposition (SVD) from a cluster of genes associated tumor sensitivity to the therapeutic agent; and
c. Averaging the predictions of one or more statistical tree models applied to the values of the metagenes, wherein each model includes one or more nodes, each node representing a metagene, each node including a statistical predictive probability of tumor sensitivity to the therapeutic agent,
thereby estimating the efficacy of a therapeutic agent in a subject afflicted with cancer.

49. A method for estimating the efficacy of a therapeutic agent in treating a subject afflicted with cancer, the method comprising:
a. Determining the expression level of multiple genes in a tumor biopsy sample from the subject;
b. Defining the value of one or more metagenes from the expression levels of step (a), wherein each metagene is defined by extracting a single dominant value using singular value decomposition (SVD) from a cluster of genes associated tumor sensitivity to the therapeutic agent; and
c. Averaging the predictions of one or more binary regression models applied to the values of the metagenes, wherein each model includes a statistical predictive probability of tumor sensitivity to the therapeutic agent,
thereby estimating the efficacy of a therapeutic agent in a subject afflicted with cancer.

50. A method of treating a subject afflicted with cancer, said method comprising:
a. Estimating the efficacy of a plurality of therapeutic agents in treating a subject afflicted with cancer by the method comprising:
(i) determining the expression level of multiple genes in a tumor biopsy sample from the subject;
(ii) defining the value of one or more metagenes from the expression levels of step (i), wherein each metagene is defined by extracting a single dominant value using singular value decomposition (SVD) from a cluster of genes associated tumor sensitivity to the therapeutic agent; and
(iii) averaging the predictions of one or more statistical tree models applied to the values of the metagenes, wherein each model includes one or more nodes, each node representing a metagene, each node including a statistical predictive probability of tumor sensitivity to the therapeutic agent;
b. Selecting a therapeutic agent having the high estimated efficacy; and
c. Administering to the subject an effective amount of the selected therapeutic agent,
thereby treating the subject afflicted with cancer.

51. The method of claim 50, wherein a therapeutic agent having the high estimated efficacy is one having an estimated efficacy in treating the subject of at least 50%.

52. The method of claim 48, wherein said tumor is selected from a breast tumor, an ovarian tumor, and a lung tumor.

53. The method of claim 48, wherein said therapeutic agent is selected from docetaxel, paclitaxel, topotecan, adriamycin, etoposide, fluorouracil (5-FU), and cyclophosphamide, or any combination thereof.

54. A method of claim 48, wherein the therapeutic agent is docetaxel and wherein the cluster of genes comprises at least 10 genes from a metagene selected from any one of metagenes 1 through 7.

55. The method of claim 48, wherein the cluster of genes comprises at least 3 genes.

56. The method of claim 48, wherein at least one of the metagenes is metagene 1, 2, 3, 4, 5, 6, or 7.

57. The method of claim 48, wherein the cluster of genes corresponding to at least one of the metagenes comprises 3 or more genes in common to metagene 1, 2, 3, 4, 5, 6, or 7.

58. The method of claim 48, wherein each cluster of genes comprises at least 3 genes.

59. The method of claim 48, wherein step (a) comprises extracting a nucleic acid sample from the sample from the subject.

60. The method of claim 48, wherein the expression level of multiple genes in the tumor biopsy sample is determined by quantitating nucleic acids levels of the multiple genes using a DNA microarray.

61. The method of claim 48, wherein at least one of the metagenes shares at least 50% of its defining genes in common with metagene 1, 2, 3, 4, 5, 6, or 7.

62. The method of claim 48, wherein the cluster of genes for at least two of the metagenes share at least 50% of their genes in common with one of metagenes 1, 2, 3, 4, 5, 6, or 7.

63. A method for defining a statistical tree model predictive of tumor sensitivity to a therapeutic agent, the method comprising:
a. Determining the expression level of multiple genes in a set of cell lines, wherein the set of cell lines includes cell lines resistant to the therapeutic agent and cell lines sensitive to the therapeutic agent;
b. Identifying clusters of genes associated with sensitivity or resistance to the therapeutic agent by applying correlation-based clustering to the expression level of the genes;
c. Defining one or more metagenes, wherein each metagene is defined by extracting a single dominant value using singular value decomposition (SVD) from a cluster of genes associated with sensitivity or resistance; and
d. Defining a statistical tree model, wherein the model includes one or more nodes, each node representing a metagene from step (c), each node including a statistical predictive probability of tumor sensitivity or resistance to the agent,
thereby defining a statistical tree model indicative of tumor sensitivity to a therapeutic.

64. The method of claim 63, further comprising:
e. Determining the expression level of multiple genes in a tumor biopsy samples from human subjects
f. Calculating predicted probabilities of effectiveness of a therapeutic agent for tumor biopsy samples; and
g. Comparing these probabilities to clinical outcomes of said subjects to determine the accuracy of the predicted probabilities,
thereby validating the statistical tree model in vivo.

65. The method of claim 64, wherein clinical outcomes are selected from disease-specific survival, disease-free survival, tumor recurrence, therapeutic response, tumor remission, and metastasis inhibition.

66. The method of claim 63, further comprising:
e. Obtaining an expression profile from a tumor biopsy sample from the subject; and
f. Determining an estimate of the efficacy of a therapeutic agent or combination of agents in treating cancer in a subject by averaging the predictions of one or more of the statistical models applied to the expression profile of the tumor biopsy sample.

67. The method of claim 63, wherein step (d) is reiterated at least once to generate additional statistical tree models.

68. The method of claim 63, wherein each model comprises two or more nodes.

69. The method of claim 63, wherein each model comprises three or more nodes.

70. The method of claim 63, wherein each model comprises four or more nodes.

71. The method of claim 63, wherein the model predicts tumor sensitivity to an agent with at least 80% accuracy.

72. A method of estimating the efficacy of a therapeutic agent in treating cancer in a subject, said method comprising:
a. Obtaining an expression profile from a tumor biopsy sample from the subject; and
b. Calculating probabilities of effectiveness from an in vivo validated signature applied to the expression profile of the tumor biopsy sample.

73. The method of claim 72, wherein said therapeutic agent is selected from docetaxel, paclitaxel, topotecan, adriamycin, etoposide, fluorouracil (5-FU), and cyclophosphamide.

74. The method of claim 48, further comprising:
d. Detecting the presence of pathway deregulation by comparing the expression levels of the genes to one or more reference profiles indicative of pathway deregulation, and
e. Selecting an agent that is predicted to be effective and regulates a pathway deregulated in the tumor.

75. The method of claim 74, wherein said pathway is selected from RAS, SRC, MYC, E2F, and β-catenin pathways.
